(19) United States
(12) Patent Application Publication
(10) Pub. No.: US 2026/0360514 A1
(43) Pub. Date: Nov. 12, 2026

(54) MULTI-DISCIPLINARY TEAM SUMMARIZATION USING MULTIMODAL GENERATIVE MACHINE LEARNING

(71) Applicant: Oracle International Corporation, Redwood Shores, CA (US)

(72) Inventors: Praphul Kumar Singh, Pleasanton, CA (US); Sagar Kalyan Gollamudi, San Diego, CA (US)

(73) Assignee: Oracle International Corporation, Redwood Shores, CA (US)

(21) Appl. No.: 19/205,103

(22) Filed: May 12, 2025

(51) Int. Cl. G16H 30/20; G06F 16/34; G06T 7/00; G06T 7/11; G10L 21/0272; G16H 10/60; G16H 20/00

(52) U.S. Cl. CPC G16H 30/20; G06F 16/345; G06T 7/0012; G06T 7/11; G10L 21/0272; G16H 10/60; G16H 20/00

(57) ABSTRACT

Techniques are disclosed herein for generating a summary for content using an AI-powered clinical digital assistant with generative models. In one aspect, a method includes: accessing a medical image and/or audio transcript data associated with a patient. A plurality of results from at least one machine learning model are obtained based on a plurality of machine learning model prompts. The plurality of results comprise for the medical image: image segmentation, anomaly detection result, and image analysis summarization, and for the audio transcript data record: dialogue segmentation result, one or more individual summarization results, and aggregated summarization result. The plurality of results are generated by providing to the one or more machine learning models, the one or more machine learning prompts corresponding to the plurality of results. Based on the results, a graphical user interface page is generated providing summarizations for the medical image and/or audio transcript data.

### FIELD

The present disclosure relates generally to generative model summarization, and more particularly, to use of multimodal generative models to generate concise and informative summaries of cross-modal inputs associated with multi-disciplinary teams (MDT).

### BACKGROUND

Multimodal large language models (LLMs) represent a significant leap in artificial intelligence, integrating capabilities to process and analyze diverse types of data, including text, images, and structured inputs like medical records. In the context of medical image analysis, these models are designed to bridge the gap between textual medical knowledge of doctors and visual data, enabling the interpretation of complex medical images alongside understanding clinical reports, patient histories, and diagnostic guidelines.

Traditional medical image analysis relied heavily on specialized algorithms tailored for specific imaging modalities, such as convolutional neural networks (CNNs) for radiology or histopathology images. While effective, these systems lacked the ability to incorporate broader contextual information, such as correlating imaging findings with patient symptoms, lab results, or treatment plans. Multimodal LLMs have several core functions that make them invaluable in medical image analysis. For example, multimodal LLMs can analyze medical images, such as X-rays, CT scans, MRIs, and histopathology slides, to identify abnormalities, diagnose conditions, and prioritize findings. Moreover, they can combine the information gleaned from medical images with other data sources, such as lab results or genetic profiles, to provide evidence-based recommendations. For instance, they can suggest appropriate imaging modalities for follow-up or predict patient outcomes based on imaging findings and clinical data. Additionally, these models can combine visual analysis with textual capabilities, by automatically generating detailed and coherent medical reports. These reports typically include descriptions of findings, clinical interpretations, and recommendations for further investigation or treatment. Overall, multimodal LLMs overcome the limitations of traditional medical image analysis by integrating vast datasets that include both textual and visual medical knowledge, allowing for a more holistic approach to diagnosis and decision-making.

The emergence of multimodal LLMs, particularly in the medical field, is fueled by advancements in deep learning architectures. One such advancement is the incorporation of transformer models, which underpin the ability of multimodal LLMs to handle cross-modal inputs. By training on massive datasets containing medical images paired with textual annotations (e.g., radiology reports or clinical notes), these models learn to recognize patterns, extract relevant features, and make accurate predictions that align with human-level expertise. In so doing, these models significantly improve medical professionals' ability to develop personalized health care plans for their patients.

### BRIEF SUMMARY

Machine learning techniques are disclosed herein (e.g., a computer implemented method, a system, non-transitory computer-readable medium storing code or instructions executable by one or more processors) for generating concise and informative summaries of cross-modal inputs associated with MDT.

In some embodiments, a computer-implemented method is provided comprising accessing a medical image associated with a patient; obtaining, based on a plurality of machine learning model prompts, a plurality of results from at least one machine learning model, the plurality of results comprising an image segmentation result for the medical image, an anomaly detection result for the medical image, and an image analysis summarization result for the medical image, wherein obtaining the plurality of results comprises: using one or more first machine learning model prompts to obtain the image segmentation result for the medical image from the at least one machine learning model, at least one first machine learning model prompt of the one or more first machine learning model prompts comprising the medical image; using one or more second machine learning model prompts to obtain the anomaly detection result for the medical image from the at least one machine learning model, at least one second machine learning model prompt of the one or more second machine learning model prompts comprising the image segmentation result; and using one or more third machine learning model prompts to obtain the image analysis summarization result for the medical image from the at least one machine learning model, at least one third machine learning model prompt of the one or more third machine learning model prompts comprising the anomaly detection result; and generating, based on the plurality of results, a graphical user interface page that provides the image analysis summarization result for the medical image.

In some embodiments, the computer-implemented method further comprises: generating, based at least on the medical image, a particular machine learning model prompt included in the one or more first machine learning model prompts; providing the one or more first machine learning model prompts to the at least one machine learning model; and receiving, from the at least one machine learning model, the image segmentation result, wherein the image segmentation result includes image segment data representing structural tissue variations depicted in the medical image.

In some embodiments, the computer-implemented method further comprises: generating, based at least on image segment data that is included in the image segmentation result, a particular machine learning model prompt included in the one or more second machine learning model prompts; providing the one or more second machine learning model prompts to the at least one machine learning model; and receiving, from the at least one machine learning model, the anomaly detection result, wherein the anomaly detection result includes a) one or more anomaly labels associated with a respective image segment in the image segment data, and b) text data describing a respective context associated with each of the one or more anomaly labels, the respective context based on a combination of each of the anomaly labels and electronic health data that is associated with the patient.

In some embodiments, the computer-implemented method further comprises: generating, based at least on one or more anomaly labels included in the anomaly detection result, a particular machine learning model prompt included in the one or more third machine learning model prompts, wherein each of the one or more anomaly labels is associated with a respective image segment that is based on the medical image; providing the one or more third machine learning model prompts to the at least one machine learning model; and receiving, from the at least one machine learning model, the image analysis summarization result, wherein the image analysis summarization result includes treatment recommendation data associated with the one or more anomaly labels, the treatment recommendation data describing a treatment option for a structural tissue variation represented by the respective image segment.

In some embodiments, the computer-implemented method further comprises: accessing an audio transcript data record associated with the patient; obtaining, using the plurality of machine learning model prompts a dialogue segmentation result from at least one machine learning model, wherein the dialogue segmentation result identifies multiple participants in a dialogue recorded in the audio transcript data record; obtaining, using the plurality of machine learning model prompts a) one or more individual summarization results comprising dialogue summarizations corresponding to each of the multiple participants identified in the dialogue segmentation result, and b) an aggregated summarization result comprising an additional dialogue summarization corresponding to the dialogue among the multiple participants identified in the dialogue segmentation result; and modifying the graphical user interface page to further provide the one or more individual summarization result and the aggregated summarization result.

In some embodiments, the computer-implemented method further comprises: generating, based at least on the audio transcript data record, a particular machine learning model prompt included in the one or more machine learning model prompts; providing the particular machine learning model prompt to the at least one machine learning model; and receiving, from the at least one machine learning model, the dialogue segmentation result, wherein the dialogue segmentation result comprises a) identification data for the multiple participants in the dialogue recorded in the audio transcript data record, b) the dialogue, and c) the additional dialogue, wherein the identification data is based on personnel data describing one or more of the multiple participants, wherein the particular dialogue and the additional dialogue are determined based on a combination of the identification data and the audio transcript data record.

In some embodiments, the computer-implemented method further comprises: generating, based at least on the dialogue segmentation result, a particular machine learning model prompt included in the plurality of machine learning model prompts; providing the particular machine learning model prompt to the at least one machine learning model; and receiving, from the at least one machine learning model, one or more of the individual summarization result or the aggregated summarization result, wherein the individual summarization result includes a respective individual summarization of respective dialogue of each participant of the multiple participants, wherein the aggregated summarization result is based on a combination of the respective individual summarization for each participant.

Some embodiments include a system that includes one or more processors; and one or more computer-readable media storing instructions which, when executed by the one or more processors, cause the system to perform part or all of the operations and/or methods disclosed herein.

Some embodiments include one or more non-transitory computer-readable media storing instructions which, when executed by one or more processors, cause a system to perform part or all of the operations and/or methods disclosed herein.

The techniques described above and below may be implemented in a number of ways and in a number of contexts. Several example implementations and contexts are provided with reference to the following figures, as described below in more detail. However, the following implementations and contexts are but a few of many.

### BRIEF DESCRIPTION OF THE DRAWINGS

The present invention will be better understood in view of the following non-limiting figures, in which:

FIG. 1 is a simplified diagram of an AI-enabled system that provides various services to users, according to various embodiments.

FIG. 2 depicts a simplified architectural diagram of an ASR computing system configured to generate transcript data, according to various embodiments.

FIG. 3 depicts a block diagram of an example computing environment of a summary generation system configured to automatically generate summaries for various content to be summarized, according to various embodiments.

FIG. 4 shows an example of content to be summarized by the computing environment described in FIG. 3, according to various embodiments. The exemplary content can include medical images, such as radiology images.

FIG. 5A is a block diagram summarizing an exemplary workflow for using an MDT Assist, according to various embodiments.

FIGS. 5B-5F illustrate various aspects of a graphical user interface and exemplary workflow for using an MDT Assist according to various embodiments.

FIG. 6 is a block diagram showing an exemplary workflow for using an MDT Assist to review and analyze medical images, according to various embodiments.

FIG. 7 is a block diagram showing an exemplary workflow for using an MDT Assist to generate MDT discussion summaries, according to various embodiments.

FIG. 8 is a flowchart illustrating a process for use of multimodal generative models to generate concise and informative summaries of cross-modal inputs associated with MDT, according to various embodiments.

FIG. 9 is a block diagram illustrating one pattern for implementing a cloud infrastructure as a service system, according to various embodiments.

FIG. 10 is a block diagram illustrating another pattern for implementing a cloud infrastructure as a service system, according to various embodiments.

FIG. 11 is a block diagram illustrating another pattern for implementing a cloud infrastructure as a service system, according to various embodiments.

FIG. 12 is a block diagram illustrating another pattern for implementing a cloud infrastructure as a service system, according to various embodiments.

FIG. 13 is a block diagram illustrating an example computer system, according to various embodiments.

### DETAILED DESCRIPTION

In the following description, for the purposes of explanation, specific details are set forth in order to provide a thorough understanding of certain embodiments. However, it will be apparent that various embodiments may be practiced without these specific details. The figures and description are not intended to be restrictive. The word “exemplary” is used herein to mean “serving as an example, instance, or illustration.” Any embodiment or design described herein as “exemplary” is not necessarily to be construed as preferred or advantageous over other embodiments or designs.

### INTRODUCTION

The present disclosure relates to systems and methods for medical image analysis using an AI-powered clinical digital assistant having generative models for summarizing cross-modal inputs such as medical images and discussions. Clinical environments, such as healthcare facilities, often include multidisciplinary teams (MDT) of healthcare providers who collaborate to provide comprehensive care to patients. Key members of an MDT team typically include doctors (e.g., surgeons, oncologists, or general practitioners who oversee the medical aspects of care), the nurses who provide day-to-day care, support, and patient education, allied health professionals (e.g., physiotherapists, occupational therapists, dietitians, and social workers who contribute their expertise to the patient's treatment plan), pharmacists, psychologists and/or psychiatrists, and case managers and/or care coordinators. The goal of an MDT is to bring together a diverse range of expertise to discuss, plan, and deliver patient care, ensuring that all aspects of a patient's condition are considered. By integrating different perspectives, MDTs ensure that all aspects of a patient's health—physical, psychological, and social—are addressed. In so doing, these collaborative decision-making efforts ensure a personalized care plan tailored to the individual needs of each patient, considers their specific medical, psychological, and social circumstances.

Despite their numerous advantages, MDT discussions, particularly when conducted online, face significant challenges when analyzing medical images such as radiology, pathology, MRIs, and CTs, and the like. These challenges stem from the inherently complex and time-consuming process of thoroughly examining medical images to identify anomalies and derive unbiased conclusions. The precision required for such analysis demands specialized knowledge and a high level of concentration from doctors and other team members involved. Consequently, this meticulous process becomes a bottleneck in MDT workflows, affecting efficiency and decision-making. Another obstacle often encountered in online MDT meetings is the summarization of discussions. These meetings involve capturing and synthesizing the diverse perspectives from specialists across various disciplines into a cohesive consensus summary. Achieving this requires a deep understanding of each expert's input to ensure the representation of all relevant information. However, the complexity of these discussions and the need for precise communication make this task resource-intensive, leading to delays in care planning and decision-making.

Several examples highlight the challenges MDT teams face. First, the interpretation of complex medical images such as radiology images (e.g., CT scans, MRIs, and X-rays), pathology images, ultrasounds, PET scans, and the like, often involves subtle details that can be overlooked without careful analysis. These images require high-resolution reviews from multiple angles to identify abnormalities, and the presence of overlapping structures or artifacts adds to the difficulty. Second, inter-specialty communication is another challenge. Doctors must effectively convey their findings to specialists outside of their field of practice, each of whom has unique focuses and terminologies. This complexity in translation can hinder clarity and collaboration among team members. With respect to generating a discussion summary that captures each expert's insights and recommendations, conventional generative models are only capable of generating generic, unstructured reports.

Another challenge is the time-intensive process of analyzing medical images in a limited timeframe during online MDT meetings. The pressure for efficient decision-making can result in incomplete evaluations or oversights, potentially compromising patient care. Additionally, forming unbiased conclusions requires integrating findings from clinical data, patient history, and input from various specialists. Subjective interpretations of medical images can introduce bias, complicating consensus-building efforts. Furthermore, logistical challenges such as real-time sharing of high-quality images during virtual meetings demand robust digital infrastructure. Technical issues, including poor resolution or lag, can disrupt the flow of discussions and hinder effective analysis.

These challenges collectively contribute to a significant bottleneck in MDT discussions, potentially delaying diagnosis and treatment decisions. The need for deep expertise, coupled with the time-intensive nature of medical image analysis (e.g., radiology analysis), makes it difficult to efficiently and effectively integrate medical findings into the broader MDT discussion. This can lead to incomplete or biased conclusions, ultimately affecting the quality of patient care and outcomes.

The disclosed embodiments seek to overcome these challenges associated with MDT discussions. Described herein are one or more generative models that perform, in real-time, summarizations of cross-modal inputs such as medical images and discussions. This solution is designed to enhance the efficiency, accuracy, and objectivity of medical image interpretations by automating key aspects of the analysis and seamlessly integrating it into MDT discussions. The one or more generative models may be LLMs and/or multimodal LLMs. The one or more generative models address challenges in analyzing complex medical images during MDT discussions by performing segmentation and analysis. Utilizing advanced computer vision techniques, the model identifies anatomical structures and regions of interest, such as tumors or lesions, and automatically detects anomalies with detailed insights contextualized by patient history and similar cases. This reduces the cognitive load on doctors and enhances inter-specialty communication. The generative model(s) synthesizes findings into unbiased conclusions by leveraging their ability to process vast amounts of data and validating results against clinical data, ensuring evidence-based recommendations tailored to the patient's condition. Moreover, recommendations are informed by best practices and guidelines derived from the latest medical literature and can be further refined based on input from the MDT. It also provides alternative treatment options, highlighting overlapping perspectives to guide collaborative decision-making. Through the seamless integration with online MDT platforms, the model(s) enable real-time image analysis and discussion during virtual meetings. Medical images can be uploaded directly into the platform and the model(s) perform their analysis and presents the results instantly, ensuring consistent information access for all team members. The medical image analysis results are presented in a structured, multi-layered summary that captures all relevant perspectives. In so doing, the decision-making process is streamlined, cognitive burden is reduced, and a comprehensive treatment planning grounded in collective expertise is generated.

The one or more generative models in the disclosure address the challenge of summarizing MDT discussions by generating a structured, multi-section summary that enhances clarity, reduce bias, and capture all relevant perspectives effectively. At least one (or the same) generative model synthesizes individual specialist's viewpoints into a unified narrative by highlighting common ground among specialists. This ensures a cohesive understanding of the patient's condition and treatment options. Additionally, the at least one (or same) generative model mitigates bias in treatment planning by focusing solely on overlapping recommendations from experts. This approach ensures that the proposed plan is balanced, reflective of collective expertise, and actionable, minimizing the influence of any single viewpoint while fostering collaborative decision-making.

In an exemplary embodiment, a computer-implemented method is provided that includes accessing a medical image associated with a patient; obtaining, based on a plurality of machine learning model prompts, a plurality of results from at least one machine learning model, the plurality of results comprising an image segmentation result for the medical image, an anomaly detection result for the medical image, and an image analysis summarization result for the medical image, where obtaining the plurality of results comprises: using one or more first machine learning model prompts to obtain the image segmentation result for the medical image from the at least one machine learning model, at least one first machine learning model prompt of the one or more first machine learning model prompts including the medical image; using one or more second machine learning model prompts to obtain the anomaly detection result for the medical image from the at least one machine learning model, at least one second machine learning model prompt of the one or more second machine learning model prompts comprising the image segmentation result; and using one or more third machine learning model prompts to obtain the image analysis summarization result for the medical image from the at least one machine learning model, at least one third machine learning model prompt of the one or more third machine learning model prompts comprising the anomaly detection result; and generating, based on the plurality of results, a graphical user interface page that provides the image analysis summarization result for the medical image.

As used herein, the terms “about,” “similarly,” “substantially,” and “approximately” are defined as being largely but not necessarily wholly what is specified (and include wholly what is specified) as understood by one of ordinary skill in the art. In any disclosed embodiment, the term “about,” “similarly,” “substantially,” or “approximately” may be substituted with “within [a percentage] of” what is specified, where the percentage includes 0.1 percent, 1 percent, 5 percent, and 10 percent, etc.

As used herein, when an action is “based on” something, this means the action is based at least in part on at least a part of the something.

### Overview of an AI-Enabled System

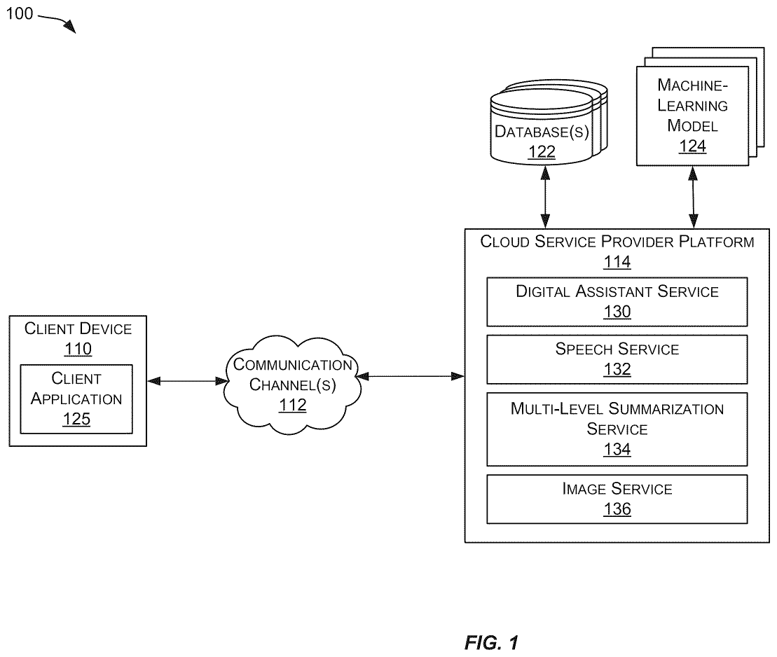

FIG. 1 is an example of an AI-enabled system 100 that includes capabilities for providing various services to users. The end-users (e.g., clinicians such as doctors and nurses) may utilize the various services provided by the cloud service provider platform 114 to perform various functions. The AI-enabled system 100 includes one or more client devices 110 (hereinafter “client devices 110”), one or more communication channels 112 (hereinafter “communication channels 112”), a cloud service provider platform 114 (hereinafter “platform 114”), one or more databases 122 (hereinafter “databases 122”), and one or more machine learning models 124 (hereinafter “models 124”). While FIG. 1 shows the databases 122 and the models 124 in one particular configuration, this is not intended to be limiting, and one or more of the databases 122 and/or one or more of the models 124 can be included separately or as part of the platform 114 and/or the cloud infrastructure in which the platform 114 is included.

The AI-enabled system 100 provides intelligent assistant services to healthcare providers such as doctors, nurses, technicians, clinicians, medical personnel, and the like. As used herein, the term healthcare provider generally refers to healthcare practitioners and professionals including, but not limited to: physicians (e.g., general practitioners, specialists, surgeons, etc.); nurse professionals (e.g., nurse practitioners, physician assistants, nursing staff, registered nurses, licensed practical nurses, etc.); other professionals (e.g., pharmacists, therapists, technicians, technologists, pathologists, dietitians, nutritionists, emergency medical technicians, psychiatrists, psychologists, counselors, dentists, orthodontists, hygienists, etc.).

Healthcare providers interact with system 100 using client devices 110. Each client device included in the client devices 110 can be any kind of electronic device that is capable of: executing applications; presenting information textually, graphically, and audibly such as via a display and a speaker; collecting information via one or more sensing elements such as image sensors, microphones, tactile sensors, touchscreen displays, and the like; connecting to a communication channel such as the communication channels 112 or a network such as a wireless network, wired network, a public network, a private network, and the like, to send and receive data and information; and/or storing data and information locally in one or more storage mediums of the electronic device and/or in one or more locations that are remote from the electronic device such as a cloud-based storage system, the platform 114, and/or the databases 122. Large healthcare organizations (e.g., a large city hospital or geographically dispersed hospital system) may have thousands, tens of thousands, or hundreds of thousands—or even more—client devices being used simultaneously for healthcare delivery in various capacities. Examples of electronic devices include, but are not limited to, mobile phones, desktop computers, portable computing devices, computers, workstations, laptop computers, tablet computers, and the like.

In some implementations, application 125 can be installed on, executing on, and/or accessed by a client device included in the client devices 110. The application 125 and/or a user interface of the application can be utilized and/or interacted with (e.g., by an end user such as healthcare providers) to access, utilize, and/or interact with one or more services provided by the platform 114. An example of an application that be installed on, executed on, and/or accessed by client devices 110 is Clinical AI Agent application (with Multi-Disciplinary Teams capabilities, e.g., MDT-Assist). The client device 110 can be configured to receive multiple forms of input such as touch, text, voice, images, and the like, and the application 125 can be configured to transform that input into one or more messages which can be transmitted or streamed to the platform 114 using one or more communication channels of the communication channels 112. Additionally, the client device 110 can be configured to receive messages, data, and information from the platform 114 using one or more communication channels of the communication channels 112 and the application 125 can be configured to present and/or render the received messages, data, and information in one or more user interfaces of the application 125.

Each communication channel included in the communication channels 112 can be any kind of communication channel that is capable of facilitating communication and the transfer of data and/or information between one or more entities such as the client devices 110, the platform 114, the databases 122, and the models 124. Examples of communication channels include, but are not limited to, public networks, private networks, the Internet, wireless networks, wired networks, fiber optic networks, local area networks, wide area networks, and the like. The communication channels 112 can be configured to facilitate data and/or information streaming between and among the one or more entities. In some implementations, data and/or information can be streamed using one or more messages and according to one or more protocols. Each of the one or more messages can be a variable length message and each communication channel included in the communication channels 112 can include a stream orchestration layer that can receive the variable length message in accordance with a predefined interface, such as an interface defined using an interface description language like AsyncAPI. Each of the variable length messages can include context information that can be used to determine the route or routes for the variable length message as well as a text or binary payload of arbitrary length. Each of the routes can be configured using a polyglot stream orchestration language that is agnostic to the details of the underlying implementation of the routing tasks and destinations.

Each database included in the databases 122 can be any kind of database capable of storing data and/or information and managing data and/or information. Data and/or information stored by each database 122 can include data and/or information generated by, provided by, and/or otherwise obtained by the platform 114. Additionally, or alternatively, data and/or information stored and/or managed by each database 122 can include data and/or information generated by, provided by, and/or otherwise obtained by other sources such as the client devices 110 and/or models 124. In some cases, the cloud service provider platform 114 and the databases 122 may be configured to securely store or transmit electronic information that is protected. Examples of protected information can include health information, financial information, personal information, or other types of protected information. Examples of secure storage or transmission of electronic information can include encryption, hashing, randomization, access authentication, or other techniques to secure electronic information.

One or more databases that are included in the databases 122 can be part of a platform for storing and managing healthcare information such as electronic health records for patients, electronic records of healthcare providers, and the like, and can store and manage electronic health records for patients of healthcare providers. Each electronic health record associated with a patient can be linked to other electronic health records associated with the patient. In some instances, the databases 122 store electronic health records as one or more data entities, such as database records, digital files, or other types of data objects. For example, the databases 122 store a particular electronic health record as one or more data entities that describe health information for a patient associated with the particular electronic health record, such as data entities that respectively describe personal information, appointment information, medical procedures (e.g., scheduled procedures, completed procedures, etc.), diagnoses, prescriptions, notes by healthcare providers, imaging results (e.g., radiology images, CT scans, MRIs, etc.), lab results (e.g., pathology, genetic testing, etc.) or other types of digitally stored health information. In some embodiments, the databases 122 include one or more electronic health records that are digitally secured data entities, such as electronic health records that include protected information for associated patients. Examples of protected information can include personal identification for a patient (e.g., name, address, etc.), health information for a patient (e.g., diagnoses, scheduled procedures, provider notes, prescriptions, etc.), financial information for a patient (e.g., insurance data, payment plan data, etc.), or other types of patient-related information that are identified in the AI-enabled system 100 as protected information. Additionally, one or more databases included in the databases 122 can be provided by, managed by, and/or otherwise included as part of a cloud infrastructure of a cloud service provider (e.g., Oracle Cloud Infrastructure or OCI). Data and/or information stored and/or managed by the databases 122 can be accessed using one or more application programming interfaces (APIs) of the databases 122.

The one or more machine learning models 124 can include but are not limited to: pre-trained machine-learning models, opensource machine-learning models, licensed machine-learning models, generative machine-learning models, transformer-based machine-learning models, and the like. The models 124 can be any kind of machine-learning model that can facilitate the providing of various services by the cloud service provider platform 114. More specifically, the one or more machine learning models 124 can include large language model (LLM), multimodal LLMs, automatic speech recognition (ASR) model, and the like. For example, in some cases, the platform 114 may generate text (e.g., summarizations, notetaking, etc.) from input data. As such, the platform 114 could utilize a dictation service in which one of the models in the one or more models 124 is a LLM used to generate the text. In another example, the platform 114 may use a multimodal LLM capable of obtaining, generating, and/or retrieving one or more results in response to one or more inputs such as one or more prompts. Prompts for obtaining or generating or retrieving results from the multimodal LLM can be obtained from or generated by or retrieved from or accessed from the client devices 110, the databases 122, the platform 114, and/or one or more other sources such as the Internet. Each prompt can be configured to cause the multimodal LLM to perform one or more tasks, which causes one or more results to be provided or generated and the like. As another example, the platform 114 could utilize an automatic speech recognition (ASR) model to convert audio into text such as a text transcript.

The platform 114, for example the Oracle Health Millenium Platform, can be included as part of a cloud infrastructure of a cloud service provider (e.g., Oracle Cloud Infrastructure or OCI). The platform 114 can be configured to communicate with, send data and information to, and receive data and information from the client devices 110 via the communication channels 112. Additionally, the platform 114 can be configured to interact with the databases 122 and the models 124 to obtain and/or receive data and information from the databases 122 and the models 124. Data and information received from the client devices 110, the databases 122, and the models 124 can be used by the platform 114 to execute tasks and perform services, such as cloud services, to subscribers (e.g., end-users) of the cloud service provider platform 114.

In the AI-enabled system 100, the cloud service provider platform 114 provides one or more services, including the cloud services, to additional computing systems included in (or in communication with) the AI-enabled system 100. For example, the cloud service provider platform 114 can provide one or more digital services to the client devices 110. The platform 114 can be configured to include various capabilities and provide various services to subscribers (e.g., end users) of the various services. In some implementations, in the case of an end user or subscriber being a healthcare provider, the healthcare provider can utilize the various services to facilitate the observation, care, treatment, management, and so on of their patient populations. For example, a healthcare provider can utilize the functionality provided by the various services provided by the platform 114 to examine and/or treat and/or facilitate the examination and/or treatment of a patient; view, edit, and/or manage a patient's electronic health record; perform administrative tasks such as scheduling appointments, managing patient populations, providing customer service to facilitate operation of a healthcare environment in which the healthcare provider practices, and so on.

The services provided by the cloud service provider platform 114 may include, but are not limited to, a digital assistant service 130, a speech service 132, a multi-level summarization service 134, and an image service 136. The services may further include authentication services, user management services, frontend services (e.g., a single-entry point to all services), and other management services. The various services may be implemented on one or more servers of the cloud service provider platform 114 using the one or more machine-learning models 124. Additionally, the various services may be provided to end-users who subscribe to the services provided by the platform 114. In a certain implementation, the services provided by the cloud service provider platform 114 may be implemented as machine-learning-based or artificial intelligence (AI)-based digital assistance tools (e.g., agentic AI-based assistants) that may be provided to end-users. For instance, the cloud service provider platform 114 can provide one or more digital assistance tools that utilize the one or more machine-learning models 124.

The digital assistant service 130 can be configured to serve as an artificial intelligence-driven (AI-driven) conversational-type interface for the platform 114 that can conduct conversations with end users (e.g., those using the client devices 110) and perform functions and/or tasks based on the information conveyed by and/or ascertained from those conversations and other sources. The digital assistant service 130 can be configured with and/or configured to access natural language understanding (NLU) capabilities such as natural language processing, named entity recognition, intent classification, and so on. In some implementations, the example conversational-type interface service can be skill-driven in which the example service includes bots that each include one or more skills for conducting conversations and performing functions and/or tasks. In some implementations, the example conversational-type interface service can be LLM-based and agent-driven in which agent(s) coordinate with LLM(s) for conducting conversations and performing functions and/or tasks. Examples of skill-driven and LLM-based and agent-driven digital assistants are described in U.S. Pat. No. 17,648,376, filed on Jan. 19, 2022, and U.S. patent application Ser. No. 18/624,472, filed on Apr. 2, 2024, each of which are incorporated by reference as if fully set forth herein. Additionally, the models 124 can include or have any size context window (i.e., can accept any number of tokens) and can be capable of interpreting complex instructions. The models 124 can be provided by, managed by, and/or otherwise included as part of the platform 114 and/or a cloud infrastructure of a cloud service provider (e.g., Oracle Cloud Infrastructure or OCI) that supports the platform 114.

In a healthcare setting in which the AI-enabled system 100 could be used, dialog is prevalent between participants of the healthcare setting. For example, at any given moment, there could be dialog or conversation among patients and healthcare providers, among multiple healthcare providers, or among other groups of participants in the example healthcare setting. As such, the digital assistant services 130 could participate in some dialogs, such as dialog or conversation of a healthcare provider, with one or more digital assistance tools (e.g., a diagnosis digital assistance tool, a recordkeeping digital assistance tool, etc.) In some cases, dialog in the example healthcare setting involves information relevant to one or more electronic health records, such as information regarding the care, treatment, observation, diagnoses for one or more patients. In some other cases, dialog involves logistics of patient care, such as what time is a physician's appointment, what procedure a nurse is to perform, what information is relevant about a particular patient for a particular appointment, and the like. In some instances, the tools provided by the digital assistant services 130 can participate in dialogs (or other forms of providing healthcare) in the example healthcare setting. For example, a healthcare provider such as an internist could use an AI-based tool to automatically organize recorded notes from an appointment with a patient. In another example, a healthcare provider such as a lab technician could use an AI-based tool to automatically file laboratory results (e.g., bloodwork) in a patient's electronic health record. In yet another example, an AI-based tool could automatically generate a health summary report for each patient having an appointment with a healthcare practitioner on a particular day, to allow the healthcare practitioner to quickly review medical information for each patient scheduled that day.

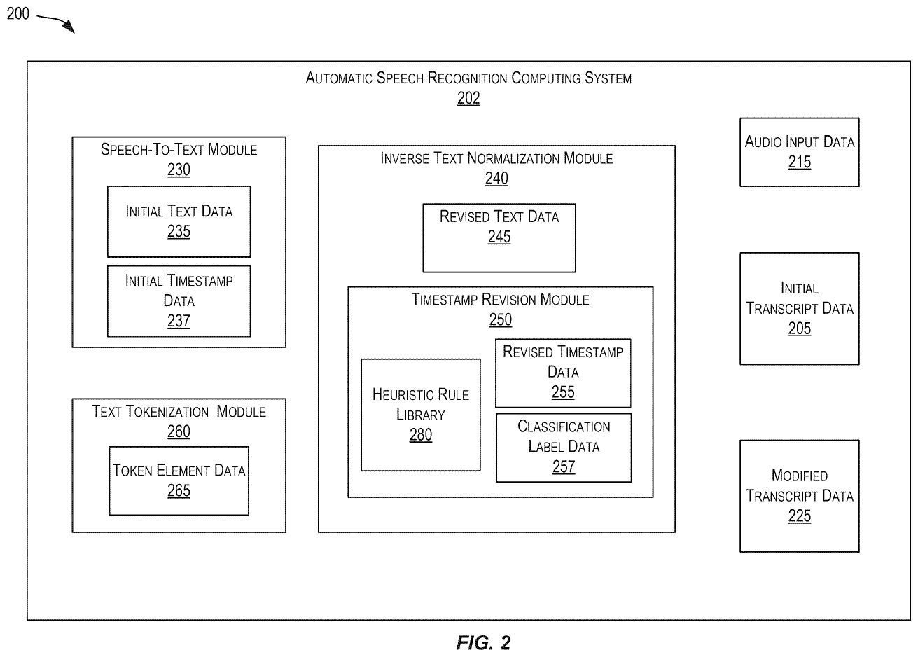

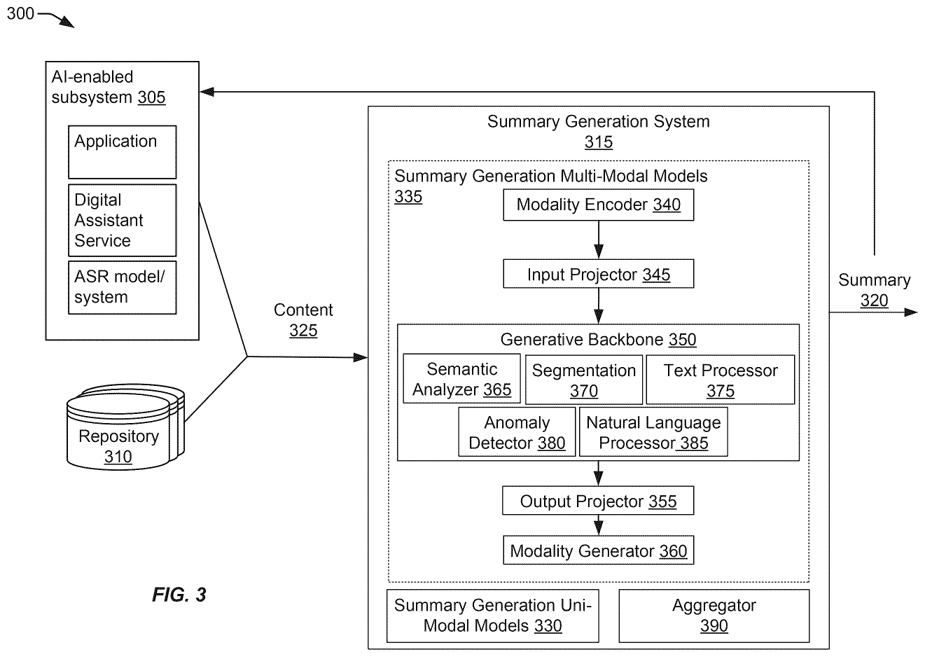

The speech service 132 can be configured to convert audio into text such as a text transcript. For example, the speech service 132 can convert an audio recording of a conversation between a healthcare provider and a patient into a text transcript of the conversation. To convert audio into text, the speech service 132 can utilize an automatic speech recognition (ASR) model (described in more detail in FIG. 2), which is included in the one or more of the machine-learning models 124. In the case that the audio is streamed to the platform 114 in the form of messages (as described above) with each message including a portion of the audio (e.g., a one second segment of the audio), in some implementations, the platform 114 and/or the speech service 132 can be configured to aggregate and combine all of the messages pertaining to the audio (e.g., all of the messages pertaining to a conversation) into audio data and/or an audio file prior to converting the audio data or audio file into text and/or a text transcript. In other implementations, the platform 114 and/or the speech service 132 can be configured to convert audio into text or a text transcript as the audio is received by the platform 114 and/or the speech service 132. The text or text transcript generated by the speech service 132 can be stored within the platform 114 and/or in another location such as in one or more databases of the databases 122, where it can be accessed by the platform 114, one or more other services of the platform 114 such as the digital assistant service 130, the multi-level summarization service 120, the image service 136, and/or the models 124. Additionally, or alternatively, the text or text transcript generated by the speech service 132 can be provided to one or more other services of the platform 114 such as the digital assistant service 130, the multi-level summarization service 134, the image service 136, and/or the models 124.

The multi-level summarization service 134 can be configured to automatically generate a structured, multi-section summary report from cross-modal inputs (e.g., audio, text, and images). To generate a summary report, the multi-level summarization service 134 can be configured to access a text transcript and generate a summary report from the text transcript. The text transcript can be a text transcript generated by the speech service 132 from an audio recording and/or a text transcript stored in and/or accessed from the platform 114, the databases 122, the models 124, and/or another location such as the client devices 110. The text transcript can correspond to an interaction between a first entity (e.g., a healthcare provider) and a second entity (e.g., a patient or another healthcare provider). In some instances, the text transcript can correspond to a multidisciplinary team meeting where two or more healthcare providers analyze and discuss the medical history of a patient in person or in a virtual meeting. In some implementations, the text transcript can correspond to an interaction between the first entity, the second entity, and one or more additional entities. The text transcript can be stored in a database or storage medium in association with the first entity, the second entity, and/or other entities. For example, the text transcript can be stored in a database of the databases 122 in association with an electronic record or electronic records of the healthcare provider and/or an electronic health record or electronic health records of a patient of the healthcare provider. In some implementations, the text transcript can be derived from an audio recording of the interaction using, for example, a machine-learning model such as an ASR model of the speech service 132. In some implementations, audio corresponding to an interaction between the first entity and the second entity can be recorded using an electronic device (e.g., a client device of the client devices 110), transmitted to the platform 114 via communications channel 112, and converted to a text transcript by the speech service 132. In some implementations, the audio recording can be of any length and can be recorded as part of a process for generating a summary report. In some implementations, to generate a summary report, the platform 114 can trigger the client devices 110 to record audio and send that recorded audio to the platform 114 and/or the databases 122, and the multi-level summarization service 134 can call the speech service 132 to generate a text transcript of the recorded audio and generate a summary report using the text transcript or portions or segments thereof. Similarly, in some implementations, to generate a summary report, the client devices 110 can record audio and send that recorded audio to the platform 114 and/or the databases 122 where the speech service 132 can receive and/or access that recorded audio, generate a text transcript, and provide the text transcript to the databases 122 and/or the multi-level summarization service 134 where it can be used to generate a summary report.

In some implementations, a summary report can be generated automatically in response to a particular trigger or indication. For example, an end user of the client devices 110 can provide an indication to the client devices 110 that a summary report is desired (e.g., a voice command). The client devices 110 can provide a message describing that indication to the platform 114 and/or the digital assistant services 130, and the platform 114 and/or digital assistant services 130 can call one or more services of the platform 114 (e.g., the speech service 132 and the multi-level summarization service 134) to begin a process for generating a summary report. In another example, a conversation in which the digital assistant service 130 is a participant can learn that an end user intends for a summary report to be generated and can call the multi-level summarization service 134 to begin a process for a generating a summary report. In another example, a button in a graphical user interface displayed on the client devices 110 can be activated and cause the platform 114 and/or a service of the platform 114 to begin a process for generating a summary report. In another example, the speech service 132 can alert the digital assistant service 130 and/or the multi-level summarization service 134 that a text transcript is available and the digital assistant service 130 and/or the multi-level summarization service 134 can access the text transcript and begin a process for generating a summary report. In some implementations, a summary report can be generated automatically at particular intervals (e.g., once per day, every time an interaction between entities concludes, and the like).

The multi-level summarization service 134 can generate a summary report from the text transcript using the models 124. To generate the summary report from the text transcript using the models 124, the multi-level summarization service 134 can access one or more prompts (e.g., one or more prompts stored in the platform 114, the databases 122, and/or in another location), and provide the one or more prompts to the models 124 to obtain one or more results (hereinafter “results”). Each prompt of the one or more prompts can include a request for or a query for or a task to be performed by the models 124 and contextual information. The contextual information can include the text transcript or portions or segments of the text transcript, information about an entity of the interaction (e.g., information about the healthcare provider, information about the patient such as information included in an electronic health record for the patient, and the like), and/or other information or records (e.g., lab results, ambient temperature, and the like). The results can include the summary report and/or portions or sections of the summary report that are combined or assembled into the summary report (e.g., by models 124 or other processing). In some implementations, the results can be processed to refine the results. For example, the one or more prompts can include a prompt which when provided to the models 124 causes the models 124 to process the summary report to refine and/or improve the summary report in the case the results include the summary report and/or process the portions or sections of the summary report to refine and/or improve the portions or sections in case the results include the portions or sections of the summary report.

Summary reports generated by the multi-level summarization service 134 can be stored within the platform 114 and/or in another location such as in one or more databases of the databases 122, where they can be accessed by the platform 114, one or more other services of the platform 114 such as the digital assistant service 130, and/or the models 124. Additionally, or alternatively, summary reports generated by the multi-level summarization service 134 can be provided to one or more other services of the platform 114 such as the digital assistant service 130 and/or the models 124. For example, the multi-level summarization service 134 can generate a summary report and provide the summary report to the digital assistant service 130 where it can be used in a conversation the digital assistant service 130 is participating in. Summary reports generated by the multi-level summarization service 134 can be stored in a database or storage medium in association with one or more entities of the interaction. For example, a summary report documenting an interaction involving a healthcare provider and a patient can be stored in a database of the databases 122 in association with or within an electronic record or electronic records of the healthcare provider and/or an electronic health record or electronic health records of the patient, where it can be accessed by the healthcare provider, the patient, another healthcare provider, and so on.

The image service 136 can be configured to analyze images (e.g., medical images) and present the results of the analysis as text such as a text transcript. For example, the image service 136 can perform segmentation and image analysis on images to identify anatomical structures, regions of interest (e.g., tumors or lesions) and anomalies. Additionally, the image service 136 may also consider clinical data (e.g., medical history, family history, lab results, genetic tests, medications, treatment plants, etc.) when generating its results. To convert image analysis into text, the image service 136 can utilize one or more machine-learning models such as a multimodal LLM. In one implementation, the platform 114 and/or the image service 136 can be configured to convert images into text or a text transcript when the image is received by the platform 114 and/or the image service 136. The text or text transcript generated by the image service 136 can be stored within the platform 114 and/or in another location such as in one or more databases of the databases 122, where it can be accessed by the platform 114, one or more other services of the platform 114 such as the digital assistant service 130, the multi-level summarization service 134, the speech service 132, and/or the models 124. Additionally, or alternatively, the text or text transcript generated by the image service 136 can be provided to one or more other services of the platform 114 such as the digital assistant service 130, the multi-level summarization service 134, the image service 136, and/or the models 124.

Although not shown, the platform 114 can include other capabilities and services such as authentication services, management services, task management services, notification services, and the like. The various capabilities and services of the platform 114 can be implemented utilizing one or more computing resources and/or servers of the platform 114 and provided by the platform 114 by way of subscriptions. Additionally, or alternatively, while FIG. 1 shows the services of the platform 114 as being separate services, one or more of the services can be combined with other services and/or be considered to be a sub-service of another service. In some cases, the digital assistant service 130, the speech service 132, the multi-level summarization service 134, and the image service 136 are accessed via one or more client applications 125 on the client devices 110 in the AI-enabled system 100. Each of the client applications 125 is configured to access one or more services provided by the cloud service provider platform 114.

The AI-enabled system 100 depicted in FIG. 1 is merely an example and is not intended to unduly limit the scope of claimed embodiments. One of ordinary skill in the art would recognize many possible variations, alternatives, and modifications. For example, in some implementations, the AI-enabled system 100 can be implemented using more or different services than those shown in FIG. 1, may combine two or more services, or may have a different configuration or arrangement of services.

### Automatic Speech Recognition System

FIG. 2 is an example of a computing environment 200 for an automatic speech recognition computing system 202 (also referred to herein as “ASR computing system 202”) configured to generated transcript data from audio. In some implementations, the ASR computing system 202 may generate timestamp data that is based on one or more combinations of token data, such as complex combinations of token elements. In FIG. 2, the ASR computing system 202 includes one or more of a speech-to-text module 230, a text tokenization module 260, or an inverse text normalization module 240 (also referred to herein as “ITN module 240”). In some implementations, the ASR computing system 202 is configured to communicate with one or more additional computing systems, such as via one or more computing networks. For example, the ASR computing system 202 could receive data from or otherwise exchange data with at least one computing system configured to provide audio data, such as from the speech service 132 on the cloud service provider platform 114 described in regard to FIG. 1.

In the computing environment 200, the ASR computing system 202 accesses audio input data 215. In some cases, the ASR computing system 202 receives the audio input data 215 from an additional computing system such as an audio source computing system configured to generate, store, or otherwise provide audio data. In some cases, the ASR computing system 202 receives the audio input data 215 from databases (such as databases 122 described with respect to FIG. 1). In some cases, the ASR computing system 202 receives the audio input data 215 from a cloud platform, such as the cloud service provider platform 114 described in regard to FIG. 1. In some cases, the ASR computing system 202 generates the audio input data 215, such as via one or more computing subsystems in the ASR computing system 202 that are configured to generate, store, or otherwise provide audio data. In some cases, the ASR computing system 202 accesses the audio input data 215 based on a combination of computing systems. For example, the ASR computing system 202 could receive unprocessed audio data from an audio source computing system and generate the audio input data 215 by applying one or more post-processing techniques (e.g., background noise removal) via a computing subsystem included in the ASR computing system 202. In some cases, the audio input data 215 is an audio recording of a conversation between healthcare providers during an MDT meeting.

Based on the audio input data 215, the ASR computing system 202 generates initial transcript data 205. In addition, the initial transcript data 205 can include one or more portions of data, such as one or more initial text data 235, initial timestamp data 237, or token element data 265. In some cases, one or more subsystems of the ASR computing system 202 generate particular portions of the initial transcript data 205. For example, the speech-to-text module 230 can access the audio input data 215. In addition, the speech-to-text module 230 generates one or more of the initial text data 235 or the initial timestamp data 237 based on the audio input data 215. In some cases, the speech-to-text module 230 can generate the initial text data 235 by applying a speech-to-text technique to convert some or all the audio input data 215 into text data. In FIG. 2, the initial text data 235 includes text data representing audio utterances that are identified in the audio input data 215. As used herein, “audio utterance” refers to a portion of audio data that indicates a spoken utterance. Examples of spoken utterances can include spoken words, spoken non-word sounds (e.g., “um” interjections, coughing, etc.), or other types of spoken sounds represented by audio data. In addition, the speech-to-text module 230 can generate the initial timestamp data 237 based on the speech-to-text technique, such as by identifying start time and end time pairs associated with respective portions of converted audio data, e.g., respective portions of text data in the initial text data 235. In some implementations, the speech-to-text module 230 can generate the initial timestamp data 237 by identifying start time and end time pairs associated with different speakers on the audio input data 215. In some implementations, the initial timestamp data 237 is generated based on naive token conversion or another technique that omits complex timestamp data for combinations of audio utterances in the initial text data 235.

In the ASR computing system 202, the text tokenization module 260 can access one or more of the initial text data 235, the initial timestamp data 237, or the audio input data 215. In addition, the text tokenization module 260 generates the token element data 265 based on one or more of the initial text data 235, the initial timestamp data 237, or the audio input data 215. For example, the text tokenization module 260 could generate the token element data 265 by arranging the initial text data 235 or the initial timestamp data 237 as a set (e.g., a sequence or other arrangement) of token elements. In some cases, each particular token element in the set indicates a particular portion of the initial text data 235 and a respective associated portion of the initial timestamp data 237. In some cases, each particular token element in the set corresponds to a particular portion of the audio input data 215, such as a particular audio utterance that is converted by the speech-to-text module 230 into a respective portion of the initial text data 235. FIG. 2 depicts the speech-to-text module 230 and the text tokenization module 260 as particular components (e.g., subsystems) of the ASR computing system 202, but other implementations are possible. For example, an ASR computing system could include a speech-to-text module that is configured to generate token element data, such as in addition to one or more of transcript data or timestamp data.

In FIG. 2, the ASR computing system 202 includes one or more of the initial text data 235, the initial timestamp data 237, or the token element data 265 in the initial transcript data 205. In some implementations, one or more of the ITN module 240 or the timestamp revision module 250 accesses the initial transcript data 205. In addition, the ASR computing system 202 generates modified transcript data 225 based on data outputs from one or more of the ITN module 240 or the timestamp revision module 250. For example, the ASR computing system 202 includes, in the modified transcript data 225, one or more of revised text data 245 or revised timestamp data 255. Based on the initial text data 235 and the associated initial timestamp data 237, the ITN module 240 generates the revised text data 245, such as via an inverse text normalization technique. The revised text data 245 includes revised text data that is based on spans (or other groups) of one or more token elements, such as token element spans identified and normalized by the ITN module 240. In some cases, the ITN module 240 provides the revised text data 245 as a data output, such as a data output that is included in the modified transcript data 225 (or otherwise accessible by the ASR computing system 202).

In the ASR computing system 202, the timestamp revision module 250 generates the revised timestamp data 255 based on one or more of the revised text data 245, the initial timestamp data 237, or the token element data 265. In addition, the ITN module 240 (or a component thereof, such as the timestamp revision module 250) can classify one or more token elements included in the token element data 265, such as generating classification label data 257 that includes respective classification labels for some or all of the token elements. Based on the respective classification labels, the timestamp revision module 250 can determine a correspondence among one or more token elements that are included in a span. For example, the timestamp revision module 250 may access, in a set of token elements included in the data 265, a span of particular token elements. In addition, the timestamp revision module 250 may determine that each particular token element in the span has respective timestamp data (e.g., a portion of the initial timestamp data 237) and a respective position in an arrangement of the token element data 265, such as sequential positions. In addition, the ITN module 240 or the timestamp revision module 250 generates, for each particular token element in the span, a respective classification label indicating an utterance type for the portions of text (e.g., from the initial text data 235 or the revised text data 245) associated with the particular token element. Examples of classification labels can include NUMBER, DATE, TIME, MEASUREMENT, FRACTION, or other suitable classification labels for types of utterances.

Based on the classification label data 257, the timestamp revision module 250 can identify one or more token spans in the token element data 265, such as span of token elements having respective classification labels that each indicate a particular utterance type. For example, the timestamp revision module 250 identifies a particular token span based on a determination that each token element in the particular span has a DATE classification label, e.g., indicating the token elements correspond to utterances involving calendar date information. In some cases, the timestamp revision module 250 can identify one or more token spans based on one or more flagged classification labels, such as flagged classification labels indicating a sequence arrangement of the token elements in the particular span. For example, the timestamp revision module 250 generates flag data for some or all the classification labels for the particular span of token elements. In some cases, the flag data indicates a position of one or more of the token elements with respect to other token elements in the particular span. For example, if the particular span of token elements is arranged as a sequence, an initial token element could have a classification label with flag data indicating an initial position in the span and additional token elements could have additional classification labels with additional flag data indicating subsequent positions in the span. In some cases, the timestamp revision module 250 identifies the particular token span based on a determination that one or more of the token elements in the particular span have a flagged DATE classification label. In the example of the DATE classification label, a first token element in the particular span could have a first classification label “DATE” in which an absence of a flag indicates a first position (such as, but not limited to, an initial position) in the sequence arrangement of the particular span. Continuing with the example DATE classification label, a second token element in the particular span could have a second classification label “DATE” in which a flag character “_” indicates a second position (such as, but not limited to, a subsequent position) in the sequence arrangement of the particular span. In FIG. 2, the timestamp revision module 250 is described as determining two types of flag data, e.g., an absence of a flag indicating an initial position and a flag character “_” indicating a subsequent (e.g., non-initial) position, but other implementations are possible, such as a timestamp revision module that is configured to determine initial position flag data, terminal position flag data (e.g., indicating a final position in a sequence), interim position flag data (e.g., indicating one or more positions between an initial position and a terminal position in a sequence), or other types of flag data. Examples of flag data can include an alphanumeric character, a Boolean data type, an absence of additional data (e.g., flagged labels compared to unflagged labels), or other types of data suitable to flag a classification label.

In FIG. 2, the timestamp revision module 250 determines the revised timestamp data 255 based on the classification label data 257, such as a combination of the classification label data 257 with one or more of the initial text data 235, the initial timestamp data 237, or the token element data 265. For example, the timestamp revision module 250 identifies, based on the classification label data 257, a token span that includes multiple token elements having classification labels indicating a particular utterance type, such as the example token span having the DATE classification label.

In addition, the timestamp revision module 250 generates a portion of the revised timestamp data 255 based on the identified token span. For example, based on the flagged classification labels associated with the identified token span, the timestamp revision module 250 identifies a first token element having an initial position in the token span and a second token element having a terminal position in the token span. In addition, the timestamp revision module 250 determines a start time of the first token element and an end time of the second token element. Based on the determined start time and end time, the timestamp revision module 250 generates a revised timestamp pair that corresponds to the identified token span. In addition, the timestamp revision module 250 includes the revised timestamp pair in the revised timestamp data 255. In some cases, the revised timestamp pair corresponds to a portion of the revised text data 245, such as particular revised text data determined by the ITN module 240 for the token elements included in the identified token span. In some cases, the timestamp revision module 250 may omit or remove interim timespan data from the revised timestamp pair, such as omitting an end time of the first token element, a start time of the second token element, or additional start or end times of additional token elements in the token span (e.g., having interim positions between the first and second token elements).

In some cases, the timestamp revision module 250 can identify one or more heuristic rule techniques to generate the portion of the revised timestamp data 255, such as identifying a particular heuristic rule based on one or more characteristics of the identified token span. For example, the timestamp revision module 250 could include (or otherwise access) a heuristic rule library 280. FIG. 2 depicts the heuristic rule library 280 as being included in the ASR computing system 202, but other implementations are possible, such as a heuristic rule library included in an additional computing system or component, which is accessible by a timestamp revision module or an ASR computing system. The heuristic rule library 280 could include multiple heuristic rule techniques associated with one or more characteristics of token elements or spans of token elements, such as a classification label characteristic. In some cases, the timestamp revision module 250 (or another component of the ASR computing system 202) identifies a heuristic rule that is associated with a particular classification label. For example, based on a determination that each token element in an identified token span has a particular classification label, e.g., the example token span having the DATE classification label, the timestamp revision module 250 identifies one or more heuristic rules associated with the particular classification label, e.g., a heuristic rule for the DATE classification label. In addition, the timestamp revision module 250 applies the identified heuristic rule to the identified token span to generate the associated portion of the revised timestamp data 255.

In some cases, the ASR computing system 202 can determine the revised timestamp data 255 with improved accuracy using heuristic rules, such as improved accuracy as compared to manual efforts to revise timestamp data. For example, the timestamp revision module 250 can use the heuristic rule library 280 to resolve (e.g., accurately generate revised timestamp data for) multiple varieties of complex token combinations that are identified in the token element data 265. Examples of complex token combinations can include one-to-one combinations (e.g., initial timestamp data for one token element is resolved to revised timestamp data for one token element), many-to-one combinations (e.g., initial timestamp data for multiple token elements is resolved to revised timestamp data for one token element), many-to-many combinations (e.g., initial timestamp data for multiple token elements is resolved to revised timestamp data for multiple token elements), or other complex combinations of token elements.

In some cases, characteristics of transcript data associated with one or more token elements can increase complexity for a token span. For example, token spans can have initial transcript data that includes one or more variations in audio utterances for describing similar types of utterances, such as “quarter until two,” “quarter to two,” “quarter before two,” or other variations in audio utterances to describe a particular time of day. In some cases, the timestamp revision module 250 can use various heuristic rules from the heuristic rule library 280 to accurately generate revised timestamp data for various complex token combinations that are identified in the token element data 265. In the ASR computing system 202, the timestamp revision module 250 determines a revised timestamp pair for each token span that is identified based on the classification label data 257. In addition, the timestamp revision module 250 generates respective portions of the revised timestamp data 255 for each revised timestamp pair. In some cases, the ITN module 240, the timestamp revision module 250, or another component of the ASR computing system 202 generates the modified transcript data 225 based on one or more of the revised text data 245 or the revised timestamp data 255. For example, the ITN module 240 could modify the modified transcript data 225 to include the revised text data 245 and the timestamp revision module 250 could modify the modified transcript data 225 to include the revised timestamp data 255. In some cases, the modified transcript data 225 could include the classification label data 257. In some implementations, the ASR computing system 202 provides some or all of the modified transcript data 225 to at least one additional system, such as the AI-enabled system 100 described in regard to FIG. 1. Based on the modified transcript data 225, the additional computing system generates one or more data outputs, such as closed captioning data, subtitle data, indexing data, or other types of data outputs that are based on transcript data.

In some cases, the timestamp revision module 250 accesses the revised text data 245 subsequent to the ITN module 240, such as accessing the revised text data 245 as a data output from the ITN module 240. In some cases, the timestamp revision module 250 accesses the revised text data 245 concurrently (or substantially concurrently) with the ITN module 240, such as accessing portions of the revised text data 245 as the portions are generated by the ITN module 240. In some implementations, the ITN module 240 generates the revised text data 245 based (at least in part) on one or more classification labels generated by the timestamp revision module 250 for token elements, e.g., classification labels that are generated concurrently (or substantially concurrently) as an inverse text normalization technique applied to the token elements by the ITN module 240. FIG. 2 depicts the ITN module 240 as including the timestamp revision module 250, but other implementations are possible. For example, an ASR computing system 202 could include an ITN module and a timestamp revision module as separate elements (e.g., subsystems).

Examples of heuristic rule techniques can include exact-match rules, span-specific rules, date-span rules, measurement-span rules, time-span rules, fraction-span rules, or additional types of rule-based techniques for additional types of spans. In some cases, the heuristic rule library 280 could include one or more heuristic rule techniques based on the examples described herein.

As an example, an exact-match heuristic rule technique can include searching for exact matches between initial text data and revised text data. In some cases, a token span that includes only one token element is identified as an exact match. If exact matches are found, the corresponding initial timestamp data (e.g., start and end time pair) is used to generate the revised timestamp data. In some cases, an exact-match heuristic rule technique can include annotating (or otherwise indicating) the matched initial text data and revised text data, such as an annotation to prevent reuse of the corresponding initial timestamp data or revised timestamp data in another portion of revised timestamp data (e.g., preventing overlapping start/end time pairs among multiple revised timestamp data portions). In some cases, an exact-match heuristic rule technique is performed prior to additional heuristic rule techniques, such as generating revised timestamp data for single-token transcript data (e.g., initial or revised text data that does not include complex combinations of token elements) prior to generating revised timestamp data for complex combinations of token elements.

As another example, a span-specific heuristic rule technique can include identifying a token element or a span of token elements that is not identified by (or otherwise is omitted from) a search for exact-match token elements, such as a search performed during an exact-match heuristic rule technique. In some cases, a span-specific heuristic rule technique is performed subsequent to performing an exact-match heuristic rule technique. For example, if there is one (e.g., only one) span of token elements remaining subsequent to performing an exact-match heuristic rule technique, then revised timestamp data for the remaining span of token elements is generated based on a start time for an initial token element in the remaining span and an end time for an initial token element in the remaining span.

In some cases, if a span-specific heuristic rule technique identifies multiple token elements or spans of token elements that are not identified by (or otherwise are omitted from) a search for exact-match token elements, one or more class-based heuristic rule techniques can be applied. In some cases, a class-based heuristic rule techniques can be identified and/or applied based on classification label data that is associated with a token element or span of token elements. For example, the timestamp revision module 250 could identify one or more class-based heuristic rule techniques based on one or more portions of the classification label data 257.

Examples of class-based heuristic rule techniques can include date-span rule techniques, measurement-span rule techniques, time-span rule techniques, fraction-span rule techniques, or additional types of class-based heuristic rule techniques for additional types of classification labels. In addition, these example class-based heuristic rule techniques can be respectively associated with classification labels such as DATE, MEASUREMENT, TIME, FRACTION, or other suitable classification labels.

As an example, a date-span heuristic rule technique can include identifying one or more portions of a token element span with a DATE classification label. In the DATE-classified span, identified portions can include respective token elements associated with initial or revised text data describing a month, a day, or a year. For example, responsive to identifying a token element associated with text data describing a month (e.g., “12”, “December”, “Dec.”), the corresponding initial timestamp data (e.g., start and end time pair) is used to generate the revised timestamp data for the DATE-classified span. In addition, responsive to identifying another token element associated with text data describing a day (e.g., “second”, “2”, “2nd”), the corresponding initial timestamp data is further used to generate the revised timestamp data for the DATE-classified span. In some cases, the text data describing the day is identified as an ordinal (e.g., “second”, “2nd”) or cardinal (e.g., “two”, “2”) date format, and the revised timestamp data is generated based on the identified date format. In addition, responsive to identifying another token element associated with text data describing a year (e.g., “2002”, “twenty oh two”), the corresponding initial timestamp data is further used to generate the revised timestamp data for the DATE-classified span. In some cases, one or more token elements in the DATE-classified span include text data describing interim words, such as text data “of” included in text data describing a phrase “second of September.” In addition, responsive to identifying a token element associated with text data describing an interim word, the corresponding initial timestamp data is further used to generate the revised timestamp data for the DATE-classified span.

As another example, a measurement-span heuristic rule technique can include identifying one or more portions of a token element span with a MEASUREMENT classification label. In the MEASUREMENT-classified span, identified portions can include respective token elements associated with initial or revised text data describing a numeric value, a separator, or a unit of measurement. For example, the MEASUREMENT-classified span is determined to include or omit a token element associated with text data describing a separator (e.g., “per”, “slash”, “a”, “each”). An example of a separator in a MEASUREMENT-classified span could be text data “an” included in text data describing a phrase “fifty milligrams an hour.” An example of a MEASUREMENT-classified span that omits a separator could be text data describing a phrase “forty-three millimeters mercury.”

Responsive to determining that the MEASUREMENT-classified span includes a token element associated with text data describing a separator, remaining token elements in the span are identified as preceding or following the token element describing the separator. In this example, a token element immediately preceding the token element describing the separator is identified as describing a first unit of measurement, such as a token element describing “milligram” in the example phrase “fifty milligrams an hour.” In addition, a token element immediately following the token element describing the separator is identified as describing a second unit of measurement, such as a token element describing “hour” in the example phrase “fifty milligrams an hour.” Further, one or more token elements preceding the token element describing the first unit of measurement are identified as describing numeric values, such as a token element describing “fifty” in the example phrase “fifty milligrams an hour.” In this example, responsive to identifying the token element describing the numeric value (e.g., “fifty”), the corresponding initial timestamp data (e.g., start and end time pair) is used to generate a first portion of the revised timestamp data for the MEASUREMENT-classified span, such as a start time and end time associated with “fifty” in the example phrase “fifty milligrams an hour.” In addition, responsive to identifying the token elements describing the separator, first unit of measurement, and second unit of measurement, the corresponding initial timestamp data is used to generate a second portion of the revised timestamp data for the MEASUREMENT-classified span, such as a start time associated with “milligrams” and an end time associated with “hour” in the example phrase “fifty milligrams an hour.”

Responsive to determining that the MEASUREMENT-classified span omits (e.g., does not include) a token element associated with text data describing a separator, the token elements in the span are identified as describing or excluding numeric values. In some cases, the token elements in the span are evaluated in a reverse order (e.g., a final token element in a span is evaluated initially). In the example phrase “forty-three millimeters mercury,” respective token elements describing “mercury” and “millimeters” are identified as excluding (e.g., not describing) numeric values. In addition, a token element describing “three” is identified as describing a numeric value. In this example, any remaining token elements preceding the token element describing “three” (e.g., remaining token elements not yet evaluated in the reverse order) are identified as describing additional numeric values, such as the token element describing “forty” in the example phrase “forty-three millimeters mercury.” In this example, responsive to identifying the token elements describing the numeric values (e.g., “three” and “forty”), the corresponding initial timestamp data is used to generate a first portion of the revised timestamp data for the MEASUREMENT-classified span, such as a start time associated with “forty” and an end time associated with “three” in the example phrase “forty three millimeters mercury.” In addition, responsive to identifying the token elements identified as excluding numeric values (e.g., “mercury” and “millimeters”), the corresponding initial timestamp data is used to generate a second portion of the revised timestamp data for the MEASUREMENT-classified span, such as a start time associated with “millimeters” and an end time associated with “mercury” in the example phrase “forty three millimeters mercury.”

In some cases, the MEASUREMENT-classified span could be determined to include multiple token elements associated with text data describing multiple separators and multiple units of measurement. An example of multiple separators in a MEASUREMENT-classified span could be text data “each” and “per” included in text data describing a phrase “five milligrams each day per pound.” An example of multiple units of measurement in a MEASUREMENT-classified span could be text data “milligrams”, “day”, and “pound” included in text data describing the phrase “five milligrams each day per pound.” In some cases, initial timestamp data for all identified separators and units of measurement is used to generate revised timestamp data, such as a start time associated with “milligrams” and an end time associated with “pound” in the example phrase “five milligrams each day per pound.”

As another example, a time-span heuristic rule technique can include identifying one or more portions of a token element span with a TIME classification label. In the TIME-classified span, identified portions can include respective token elements associated with initial or revised text data describing a numeric value or a time suffix. For example, responsive to identifying a token element associated with text data describing a time suffix (e.g., “oclock”, “pm”, “gmt”), the corresponding initial timestamp data (e.g., start and end time pair) is used to generate a first portion of the revised timestamp data for the TIME-classified span, such as a start time associated with “pm” and an end time associated with “t” in the example phrase “quarter past two pm gmt.” In this example, the first portion of the revised timestamp data is described as including revised timestamp data for token elements associated with “pm” and “gmt” but other implementations are possible. For example, a first time suffix (e.g., “pm”) could have first revised time data and a second time suffix (e.g., “gmt”) could have second revised time data. In addition, one or more token elements in the TIME-classified span are identified as being associated with text data describing a numeric value. For example, responsive to identifying a token element associated with text data describing a numeric value (e.g., “quarter”, “past”, “two”), the corresponding initial timestamp data is used to generate a second portion of the revised timestamp data for the TIME-classified span, such as a start time associated with “quarter” and an end time associated with “two” in the example phrase “quarter past two pm gmt.”

As another example, a fraction-span heuristic rule technique can include identifying one or more portions of a token element span with a FRACTION classification label. In the FRACTION-classified span, identified portions can include respective token elements associated with initial or revised text data describing an integer, a numerator, or a denominator. For example, responsive to identifying a token element associated with text data describing an integer (e.g., “twenty”, “one”), the corresponding initial timestamp data (e.g., start and end time pair) is used to generate a first portion of the revised timestamp data for the FRACTION-classified span. As an example, the first portion of the revised timestamp data could include a start time associated with “twenty” and an end time associated with “one” in the example phrase “twenty one and two thirds.” In addition, responsive to identifying additional token elements respectively associated with text data describing a numerator and a denominator, the corresponding initial timestamp data is used to generate a second portion of the revised timestamp data for the FRACTION-classified span, such as a start time associated with “two” and an end time associated with “thirds” in the example phrase “twenty one and two thirds.”

In some cases, identified portions of a FRACTION-classified span can include one or more token elements associated with initial or revised text data describing one or more interim words, such as a token element associated with “and” in the example phrase “twenty one and two thirds.” In addition, the corresponding initial timestamp data of the token element for the interim word could be included in a particular one of the first or second (or additional) portions of the revised timestamp data. Continuing with the example phrase “twenty one and two thirds,” initial timestamp data associated with “an” could be included in the second portion of the revised timestamp data, such as a start time associated with “and” and an end time associated with “thirds” in the example phrase. However, other implementations are possible, such as using initial timestamp data for one or more interim words in revised timestamp data for an integer or omitting initial timestamp data for one or more interim words from revised timestamp data.

### Summary Generation System

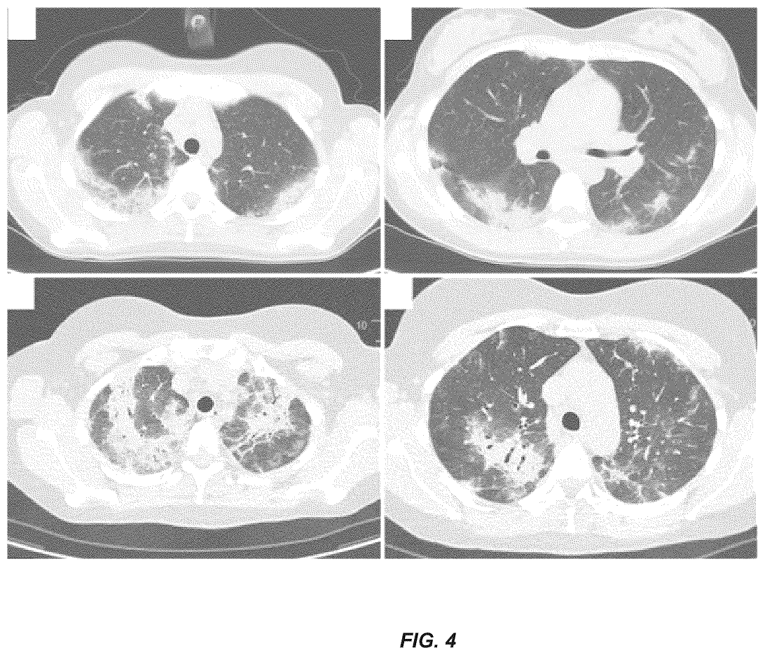

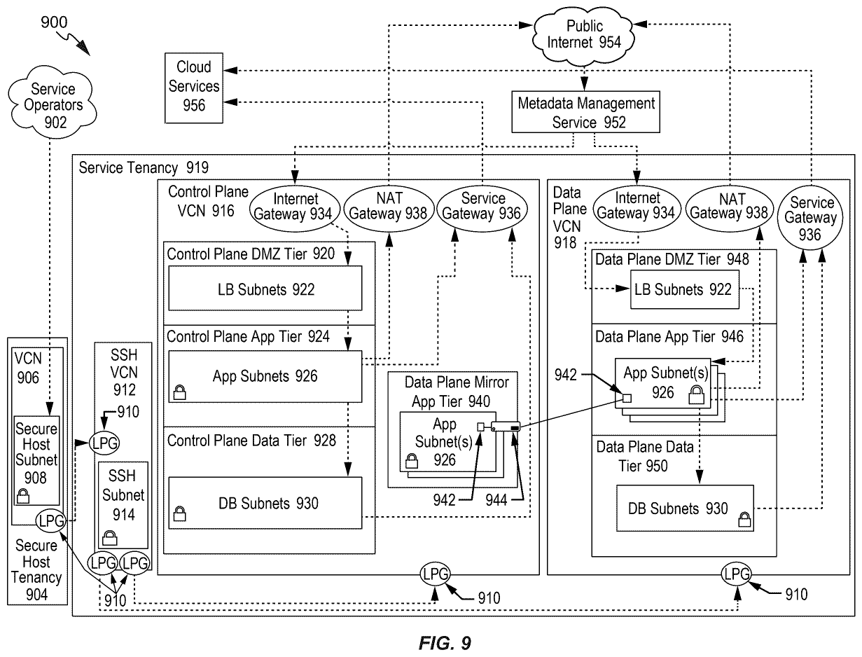

FIG. 3 depicts a computing environment 300 configured for summarizing cross-modal inputs such as medical images and discussions into a concise synthesis of key information and insights extracted from the inputs, tailored to a specific context and purpose (e.g., review of patient's medical history via a MDT). The summary generation system 305 may be implemented using software only, hardware only, firmware only, or any combination of hardware, software, and/or firmware. In some instances, the computing environment 300 is part of an Infrastructure as a Service (IaaS) cloud service (described in more detail with respect to FIGS. 9-13) and the systems and subsystems can be implemented as part of the IaaS by leveraging the scalable computing resources and storage capabilities provided by the IaaS provider to process and manage large volumes of data and complex computations. This setup can allow the summary generation system 305 to deliver real-time, responsive interactions while ensuring high availability, security, and performance scalability to meet varying demand levels. For example, the summary generation system 305 can be used to summarize cross-modal inputs as part of the intelligent assistant services offered via IaaS to healthcare providers (e.g., client application 125 and digital assistant service 130 from FIG. 1—Clinical AI Agent).

As depicted in FIG. 3, the computing environment 300 comprises subsystems, repositories, and models including but not limited to an AI-enabled subsystem 305 (e.g., the AI-enabled system 100 described with respect to FIG. 1), content repositories 310, and summary generation subsystem 315. Each subsystem can be understood to include an execution of one or more processes and/or programs implemented with software, hardware, and/or firmware within a system (e.g., as described with respect to FIGS. 9-13). Moreover, it should be understood that the one or more processes and/or programs can be executed as part of an iterative process that ultimately generates a summary of content. Iteration or an iterative process being the process of repeating a set of instructions or steps multiple times or cycles. For example, a set of instructions or steps may be executed for generating one or more summaries and repeatedly executing the set of instructions or steps multiple times or over multiple cycles results in the generation of multiple summaries (typically different summaries), i.e., a set of summaries. Each cycle of the set of instructions or steps may be executed serially, or multiple cycles of the set of instructions or steps may be executed in parallel.

The summary generation subsystem 315 may be implemented using various configurations. In certain embodiments, the summary generation subsystem 315 may represent a computing system of an entity (for e.g., an organization, an enterprise, or an individual) that provides summary generation functionality to its users. In other embodiments, the summary generator subsystem 315 may be implemented on one or more servers or virtual machines of a cloud provider network and its summary generation services may be provided to subscribers of cloud services on a subscription basis. The functionality for summary generation, as described in this disclosure, may be offered as part of the service (e.g., IaaS) via AI-enabled subsystem 305. For example, a user can subscribe to the service to generate a summary 320 of cross-modal inputs (i.e., content 325) and the summary 320 can be provided to the user via AI-enabled subsystem 305. As part of generating the summary, in certain examples, the service may also render the summary 320 on a display of a computing device as part of a UI for the requesting user as described herein.

The computing environment 300 depicted in FIG. 3 is merely an example and is not intended to unduly limit the scope of claimed embodiments. One of ordinary skill in the art would recognize many possible variations, alternatives, and modifications. For example, in some implementations, the computing environment 300 can be implemented using more or fewer subsystems, repositories, and models than those shown in FIG. 3, may combine two or more subsystems, repositories, and models, or may have a different configuration or arrangement of subsystems, repositories, and models.

The summary generation system 315 is configured to summarize cross-modal inputs (i.e., content 325) from the AI-enabled subsystem 305 (e.g., the speech service 132, the digital assistant service 130, the multi-level summarization service 134, and/or the image service 136) and/or repositories 310 in various ways. For instance, in one approach, the summary generation system 315 may receive, obtain, or be directed to (all of which are considered means of accessing the cross-modal inputs) the content 325 via an application on a user device (e.g., Clinical AI Agent application 125 having Multi-Disciplinary Teams capabilities described with respect to FIG. 1). In other instances, the summary generation system 315 may receive, obtain, or be directed to the content 325 from a digital assistant service (such as digital assistant service 130 described with respect to FIG. 1). In other instances, the summary generation system 315 may receive, obtain, or be directed to the content 325 from an ASR model or system (e.g., automatic speech recognition computing system 202 described with respect to FIG. 2). In other instances, the summary generation system 315 may receive, obtain, or be directed to the content 325 from one or more repositories 310 (e.g., databases 122 described with respect to FIG. 1). A user may interact with the summary generation system 315 using a user device (e.g., client devices 110 described with respect to FIG. 1) that is communicatively coupled to the summary generation system 315, possibly via one or more communication networks. The user device may be of various types, including but not limited to, a mobile phone, a tablet, a desktop computer, and the like. The user may use an application to provide the cross-modal inputs (i.e., content 325) to be summarized by the summary generation system 315. In other instances, the summary generation system 315 may receive, obtain, or be directed to the content 325 via a cloud service or a third-party system as described in further detail with respect to FIGS. 9-13).

The summary generation system 315 is configured to generate a summary for various types of content (i.e., cross-modal inputs), including, but not limited to, documents, webpages, news articles, research papers, hospital notes, electronic hospital records, text transcripts, audio recordings, images, and so on. In some examples, the content 325 comprises one or more audio recordings or a derivative thereof (e.g., text transcript or text data) generated from a conversation between users of an application. For example, the content 325 may include audio recordings or text data generated from conversations between healthcare providers during an MDT meeting. The audio recordings or text data from the conversations may be provided as the content 325 to the summary generation system 315 in real-time as the conversation between healthcare providers in the MDT meeting occur or the audio recordings or text data from the conversations can be generated, stored (e.g., repositories 310), and provided as the content 325 to the summary generation system 315 subsequent to occurrence of the MDT meeting. Provided in Example 1 is a non-limiting example of content 325 including a text transcript or text data generated from a conversation between healthcare providers during an MDT meeting using an application, an audio recording, an ASR model/system, or any combination thereof, as described in detail with respect to FIGS. 1 and 2.

### Example 1: Example Content to be Summarized

Speaker_0: Welcome to 80M the Emergency Medicine channel Today we are going to discuss
a case about a 55 year old female who came to ER with history of multiple episode of loose
tools and vomiting following history of food intake from outside Shall we begin
Speaker_1: Yes
Speaker_0: So um this was a 50 year old female who came to ER with history of um multiple
episodes of loose tools and vomiting uh following food intake from outside So initially uh for
patient was on initial assessment patient was conscious oriented and was able to complete full
sentence coming to the airway airway was patent and uh there was no secretions Tonsils was
also normal Breathing but air entry was bilaterally equal and respiratory rate of rate was 20 per
minute with a saturation of 98% maintaining in room air Circulation BP of 120 over 70 mm
Hg and the pulse rate of 74 per minute and all peripheral pulses were palpable also And
disability wise GCS was full score with pupil bilateral equally reacting and temperature was
expose her temperature was normal And GRBS was uh 85 milligram per deciliter coming to
the assessment of primary survey the patient was uh history was uh she was complaining of uh
multiple episodes of loose tools and vomiting so we return a VBG at the that point of time And
in VBG itself uh her potassium was showing a low potassium of 2.1 with uh a sodium 1 28
And also uh other than that his uh her pH was 7.53 with a PCO2 of 40 and the bicarbonate of
33.3 Uh her creatinine value was 0.8
Speaker_1: So what is the VBG impression
Speaker_0: VBG is showing a uh alkalosis um um metabolic alkalosis mostly can be attributed
to the multiple episode of loose tools and vomiting
Speaker_1: So um vomit vomiting mostly will cause metabolic alkalosis and along with the
patient is having only a potassium of 2.1 so that also can cause metabolic alkalosis Metabolic
alkalosis metabolic alkalosis
Speaker_0: Metabolic alkalosis
Speaker_1: can cause hypokalemia and hypokalemia can cause metabolic alkalosis
Speaker_0: Metabolic alkalosis Okay
Speaker_1:
Speaker_0: And also uh we did an ECG at that point of time
Speaker_1: Why did you do ECG
Speaker_0: So initially itself VBG was taken VBG was showing a potassium of uh 2.1 so you
want to uh know whether
Speaker_1: So any potassium less than 2.5 is severe hypokalemia so we need to look for any
ECG changes
Speaker_0: ECG changes
Speaker_1: Okay
Speaker_0: Uh so ECG was showing U waves with ST depression in 2 3 AVF and T inversion
V2 to V6
Speaker_1: What all are the hypokalemic ECG changes you expect
Speaker_0: So ECG in ECG mainly initial uh changes we can see U waves then we can see T
flattening then ST depression can can be there then we can uh see uh biphasic ST depression
uh T T inversion can be there so it will uh show us biphasic wave then we can also see
prolongation of PR then uh also like tall P waves also can be see
Speaker_1: Okay
Speaker_0: Okay So uh for the uh coming to the secondary survey we
Speaker_1: So did you start correction
Speaker_0: Yeah yeah So initially itself um we started correction as uh ECG changes was
there and VBG was also showing the same We started in the correction with uh we initially
itself we put an external jugular uh IJV line was put and uh we started with the correction Like
uh we initially started with um 40 milliequivalent in 500 ml NS which was given over 4 hours
this was given uh repeatedly for uh two was uh two times we have given and we have also sent
uh sent for the uh all routine investigation include all electrolytes and also urine potassium uh
was also said
Speaker_1: Okay So this patient's potassium uh thinking that this is a lab potassium of 2.1 so
how much will be the body be deficient in
Speaker_0: So
Speaker_1:
Speaker_0: Uh um patient uh body uh patient can be deficient like uh for more than a uh uh 1
000 milliequivalent Speaker_1: Almost 1,000 milliequivalent So uh if the patient's potassium
value is reducing by 0.3 that means patient's body itself is deficient in almost 100
milliequivalents And if the potassium value is reducing like if you are expecting potassium to
3.5 and if you're seeing it as around 2.5ish range that means that patient is deficient in more
than 300 milliequivalents So this is 2.1 so it can be if you are uh keeping 4 as a baseline so a
patient might be deficient in more than 600 600 to almost 1,000 So uh will this 40
milliequivalents be Speaker_0: Okay
Speaker_1: 40 won't be enough but uh as we never had the uh lab potassium also and uh we
sent for that also So how much can you give through a peripheral line
Speaker_0: Peripheral line mostly we will be placing 20 so how much can we give maximum
Speaker_1: Maximum we can give up to is uh uh 20 to 40 milliequivalent through peripheral
Speaker_0: 20 to 40 milliequivalent
Speaker_1: Better to keep it as 20
Speaker_0: 20 So 20 uh gauge cannula 20 milliequivalents in uh normal saline over 4 to 5
hours
Speaker_1: Okay And if we have a external jugular vein
peaker_0: 40
Speaker_1: 40 Okay So uh this patient is you told it is deficient in 1,000 milliequivalents so
can you correct this 1,000 milliequivalents over 1 day
Speaker_0: So we have to correct it over uh over the days uh the next day we can
Speaker_1: So how mu- how- what is the maximum you can correct in a day
Speaker_0: We can correct up to 100 to
Speaker_1: You can correct up to 240 milliequivalents can be corrected If you have a um
central line you can correct over uh 5 hours So if you're giving first 100 then another 100 and
40 so maximum up to 240 you can give Speaker_0: Okay
Speaker_1:
Speaker_0: So uh her history is she is a known case of type 2 diabetes So she had a history of
food intake from outside and uh following it she developed multiple episode of loose tool and
vomiting and uh then on then then again she went to nearby hospital and uh at that point of
time she developed constipation
Speaker_1: Okay
Speaker_0: And uh uh they sent the routine investigation and uh treatment for gastroenteritis
was given from that hospital uh and as she was complaining of constipation following multiple
episode of loose tools and vomiting they did a USG abdomen there and USG abdomen was
showing uh like mildly thickened uh small bowel loops in right iliac fossa and lum- lumbar
region suspecting of infective inflammatory etiology
Speaker_1: Something like infective colitis
Speaker_0: Infective colitis And other than that uh she was normal and along with this uh they
noticed that she was having this much of uh low potassium value and she was referred to our
hospital for further manage- So why is she having constipation now
Speaker_1: So uh the thing is that uh low potassium value itself can cause uh um paralysis
paralytic uh which in- in-
Speaker_0: Initially she had a gastroenteritis
Speaker_1: Gastroenteritis Uh because of the gastroenteritis she went hypokalemia
Speaker_0: Hypokalemia
Speaker_1: Hypokalemia
Speaker_0: Hypokalemia induced paralysis paralytic ileus occur
Speaker_1: Okay
Speaker_0: So um uh so she uh she some other than that her medication says she was taking
metformin and glimepiride for uh type 2 diabetes mellitus and uh she was there was no allergy
history and uh events was same as follows So uh we uh sent the initially itself we sent for the
routine investigation
Speaker_1: So this patient will require potassium correction
Speaker_0: Okay So uh we have IV correction How will you correct potassium orally
Speaker_1: Oral correction we can uh potassium chloride syrup is there we can correct it uh
like we can correct up to uh potassium chloride can be given uh 20 to up to 20 milligram more
than 20 can be given uh
Speaker_0:
Speaker_1: How how much ml will you give
Speaker_0: Uh 30
Speaker_1: If you're giving syrup KCL uh if you are giving 15 ml if we are putting a 15 ml T
ID that is a very irritant solution so if you are giving we need to dilute in water or better in
coconut water Coconut water will contain potassium so either in water or coconut water we
need to dilute 15 ml of KCL that we should give th- three ti- if you are giving three times a
day how much will that 15 ml contain
Speaker_0: So it 15 milli- uh 15 ml contain up to uh 40 millieq-
Speaker_1: 20 milliequivalent
Speaker_0: 15 ml is 20 milliequivalents So if you are giving 15 ml in one glass of water TID
so that means it will be 20 + 20 + 20 so we will be giving almost
Speaker_1: 60
Speaker_0: 60 milliequivalents orally Then if you are gi- putting a uh IJV or a central line then
we can correct through that also So if you are adding the total we need to add the oral
corrections also
Speaker_1: Okay
Speaker_0: So uh we sent the investigations where in which it was uh shown that she uh she
was having a uh magnesium of 1.5 with um serum ketones trace and potassium of uh 2. 2.1
and also her phosphorus value was 1.9 and
Speaker_1: How's her CBC CRP and
Speaker_0: CRP was 50 and total count was uh 5. 5,000
Speaker_1: Okay okay
Speaker_0: So we we are suspecting a uh gastroenteritis induced uh electrolyte imbalance
Speaker_1: Because of the depletion patient might have lost potassium
Speaker_0: Okay And uh along which is uh because of that only serum ketones became
Speaker_1: Uh because of the depletion
Speaker_0: Depletion and uh reduction in intravascular volume serum ketones also become
Speaker_1: Trace positive
Speaker_0: Okay So we have uh this patient is having hyperphosphatemia hypermagnesemia
and hypokalemia we have these three values what will you correct first
Speaker_1: So we have to correct uh magnesium value and phosphorus values before
correcting potassium because as uh correction of potassium as magnesium and phosphorus
will uh following which it will be the potassium will get corrected or else the how much we if
we give continuous or potassium also it won't get corrected because as other two uh
magnesium is uh required for correction of potassium
Speaker_0: So actual magnesium do in the body
Speaker_1: Magnesium is mainly seen in uh in- intra mostly 99% of magnesium is seen in
bones and the 1% is Speaker_0: What magnesium does to the potassium is uh there will be
renal loss of potassium So if there is presence of magnesium it will prevent the renal uh uh ex-
excretion and it will help in reabsorption of the potassium So if magnesium is deficient that
reabsorption will not happen So uh so we have to give magnesium
Speaker_1: Okay So uh what was the phosphorus value in this patient
Speaker_0: 1.9
Speaker_1: 1.9 So how will you correct this phosphorus
Speaker_0: This can be corrected with the oral correction of uh phosphorus Phosphorus uh
sashes are available we can correct it orally which will help with the
Speaker_1: If phosphorus value is coming less than 2 you can correct orally if it is coming less
than 1 we will have to give injection
Speaker_0: Injection So how is the injection available
Speaker_1: So uh
Speaker_0: in- injection of phosphorus is available with potassium potassium phosphorus
combination is there
Speaker_1: Pot-phos
Speaker_0: Pot-phos combination we can correct it like uh we can take uh depending upon
how much is the phosphorus value we will uh take according to it and correct it 5 ml or uh 5 or
3 to 5 ml can be given in 100 ml NS or
Speaker_1: So mostly it will be uh pot-phos is coming as a 15 ml injection So 15 ml in that 15
ml there will be uh 45 milliequivalents of phosphorus and there will be 20 milliequivalents
each of potassium also So uh uh so each 5 ml will contain uh at- at least 20 milliequivalents of
potassium also So uh while we are correcting potassium with uh phosphorus we need to be uh
uh correcting it very slowly we shouldn't use the entire one uh ampule of injection
Speaker_0: At least we have to take only 1/3 of that and we need to dilute in NS and we will
have to give slow IV over 5 to 6 hours uh under cardiac monitoring
Speaker_1: Okay
Speaker_0: And uh here we corrected phosphorus uh via oral correction and uh magnesium
and potassium was corrected uh through IV correction and uh other than that we also give her
IV fluids also as uh she was dehydrated
Speaker_1: Dehydrated
Speaker_0: Okay And uh during um the course in stay in the hospital her initially her
phosphorus and magnesium values were corrected and followed by uh her potassium values
also got corrected and during the days her serum ketones also became negative and uh uh her
CRP levels also came down
Speaker_1: And what antibiotic did you give her
Speaker_0: Ceftriaxone antibiotic was given as a coverage for the gastroenteritis and her uh
constipation was also relieved She was able to uh uh
Speaker_1: pass stools and potassium also came correct
Speaker_0: correct
Speaker_1: Okay
Speaker_0: Anything else
Speaker_1: What are the other causes of hypokalemia
Speaker_0: So this patient it is very clear because of the GI loss
Speaker_1: GI loss
Speaker_0: So other than that uh potassium hypokalemia can be because of decreased uh
intake of potassium then there is like increased um shift of potassium to cells which can be
occur due to diuretic use uh uh insulin insulin insulin use then it can be used to beta agonist
then it can be due to alkalosis alkalosis
Speaker_1: So first one is because of reducing Next is the shift hypokalemia
Speaker_0: Shift hypokalemia Uh which include um includes insulin then beta agonist then
alkalosis induced uh shift can be there
Speaker_1: Shift of potassium
Speaker_0: Then can be hypokalemic periodic paralysis induced then increase can be either
due to increased uh RBC production can also cause uh shift of the potassium then there is uh
paradoxical uh hypokalemia can occur Then uh like uh antipsychotics then barium cesium can
also cause shift of the uh potassium Then uh increased GI loss like in this condition can be
there then increased renal loss because of the diuretics can be there Uh other than that renal
tubular acidosis or uh polyuria
Speaker_1: That is a diseases condition which can cause hypokalemia
Speaker_0: Renal
Speaker_1: Renal tubular acidosis type 2 then
Speaker_0: Then uh Liddle syndrome Bartter syndrome Gitelman syndrome these can also
Speaker_1: Liddle syndrome will usually cause hyperkalemia Bartter's and Gitelman
syndrome will cause hypokalemia
Speaker_0: Okay Then hyperaldosteronism Cushing syndrome can cause uh potassium loss
Speaker_1: Okay
Speaker_0: These are the main conditions where potassium hypokalemia is seen So uh how
much days did you take to uh uh correct the potassium to normal
Speaker_1: Uh we corrected the normal by 4 days
Speaker_0: Okay okay okay
Speaker_1:
Speaker_0: Then uh did the patient get discharged
Speaker_1: Yeah Patient got discharged patient was fine
Speaker_0: Okay Thank you

In some examples, the content 325 comprises one or more images or a derivative thereof (e.g., image regions or patches) generated from an imaging device and/or one or more computer vision tools. For example, the content 325 may be medical images (e.g., radiology, pathology, MRIs, and CTs, and the like) generated from medical imaging equipment. In another example, the content 325 may be embeddings for patches of an image generated from a computer vision tool. An example of one or more images, specifically radiology images, are shown in FIG. 4. The summary generation system 315 accesses and processes the content 325 to generate a summary that accurately and properly describes the content 325. The transcript provided above, and images shown in FIG. 4 are merely examples of transcripts and images that may be summarized by summary generation system 315 and are not intended to unduly limit the scope of claimed embodiments. One of ordinary skill in the art would recognize many possible variations, alternatives, and modifications. For example, in some implementations, the content relates to a business discussion, an educational lecture, non-medical images, etc.

The summary generation system 315 utilizes one or more summary generation models to generate one or more summaries for the content 325. The one or more summary generation models comprise one or more machine learning models. In the specific context of this disclosure, the one or more machine learning models may be one or more generative models. A generative model is a machine learning model that is capable of generating new data instances based on the data used to train the model. A generative model may be referred to as a “generative artificial intelligence (AI) model.” Generative models learn the underlying distribution of the training data, enabling them to produce new instances of data that share properties with the original dataset. This capability makes them particularly useful in a variety of applications, including image and voice generation, text or code synthesis, and more sophisticated tasks like unsupervised learning, semi-supervised learning, and domain adaptation.

One type of generative model is a large language model (LLM) (e.g., a summary generation unimodal model 330). Large language models are designed to understand, generate, and interpret human language by processing extensive collections of data. The foundational architecture behind large language models is the transformer network, a type of neural network that excels in handling sequential data such as text. Unlike architectures, such as recurrent neural networks (RNNs) or long short-term memory networks (LSTMs), transformers do not process data in order. Instead, they leverage parallel processing to analyze entire text sequences simultaneously, significantly improving efficiency and reducing training times and inference latency times.

A mechanism that enables transformers to handle complex language tasks is self-attention. This mechanism allows the model to weigh the importance of different words within a sentence or sequence regardless of their position. For instance, in processing the phrase “The cat sat on the mat,” the model can directly associate “cat” with “mat” without having to process the intermediate words sequentially. This ability to understand the context and relationships between words in a sentence is what makes transformer networks adept at language tasks. The self-attention mechanism assigns scores to relationships between words, highlighting the most relevant connections, so the model can focus on the most informative parts of the text.

Transformers are composed of multiple layers containing a multi-head, self-attention mechanism and a position-wise, feed-forward network. Within the architecture of transformer models, the multi-head, self-attention mechanism and position-wise, feed-forward network function in concert to process input data. The multi-head, self-attention mechanism is designed to enable parallel processing of input sequences, allowing the model to simultaneously evaluate the importance of different segments of the input relative to each other. This mechanism operates by generating multiple sets of query, key, and value vectors for each element in the input sequence through linear transformation. The relevance of each element to every other element is calculated using a scaled dot-product attention function that computes the attention scores by taking the dot product of the query vector with the key vectors, dividing each by the square root of the dimension of the key vectors to scale the scores, then applying a SoftMax function to obtain the weights for the value vectors. The scaled dot-product attention function is applied independently by each head in the multi-head self-attention mechanism. The outputs of these heads are then concatenated and linearly transformed, allowing the model to capture information from different representation subspaces.

Following the multi-head, self-attention mechanism is the position-wise, feed-forward network. This component comprises two linear transformations with a non-linear activation function in between. Each element of the input sequence, now enriched with context by the self-attention mechanism, is processed independently through the same feed-forward network. The first linear transformation increases the dimensionality of the input, allowing for a richer representation space. The non-linear activation function introduces the capability to capture non-linear relationships within the data. The second linear transformation then reduces the dimensionality back to that of the model's hidden layers, preparing the output for either further processing by subsequent layers or final output generation. This sequence of operations is applied to each position in the sequence, so the model can learn complex patterns across different parts of the input data without relying on the sequential processing inherent to previous architectures, such as RNNs or LSTMs.

Integrating these components within the transformer architecture facilitates the model's ability to understand and generate human language by leveraging both the global context provided by the self-attention mechanism and the local, position-specific transformations applied by the feed-forward networks. Through the repetitive stacking of layers, transformers achieve a depth of representation that allows for the processing of linguistic information across varying levels of complexity.

Another type of generative model is a large multimodal model (LMM) (e.g., a multimodal model 335). A large multimodal model is an advanced machine learning model capable of processing and generating data across multiple modalities, such as text, images, audio, and video. These models integrate diverse datasets during training to learn the underlying distribution of different data types, enabling them to produce outputs that reflect a comprehensive understanding of the input data. These models can be used for applications such as image captioning, text-to-image generation, image-to-text generation, visual question answering, and more, where understanding the relationship between different data types is crucial. By leveraging diverse datasets during training, large multimodal models learn to create coherent and contextually relevant outputs across various modalities, enhancing their utility in complex, real-world scenarios.

The architecture of large multimodal models combines elements from different neural network designs to handle diverse data types effectively. For example, convolutional neural networks (CNNs) are often used for processing visual data, while transformer networks handle textual data, enabling the model to extract and synthesize features from both images and text. This integration results in outputs that accurately represent the input data, reflecting a deep understanding of both modalities. The transformer architecture, known for its ability to manage sequential data, is frequently adapted to work alongside CNNs, allowing these models to benefit from the strengths of each neural network type. In some instances, the architecture of large multimodal models includes a modality encoder 340, an input projector 345, a generative backbone 350, an output projector 355, and a modality generator 360.

Modality encoder 340 is responsible for transforming raw data from different modalities into representations that can be understood and processed by a generative model. Similar to tokenizers and encoders used in unimodal generative models, modality encoders process input data and convert it into a format suitable for subsequent processing by the multimodal generative model. Each modality encoder is designed to extract and encode specific features from its respective input data type. For example: Text encoders extract semantic representations from textual inputs; Image encoders capture visual features from images; and Audio encoders process audio signals to derive meaningful representations. By employing specialized encoders for each modality, this component ensures that data from text, images, audio, video, and other modalities is effectively prepared for integration and further processing.

The input projector 345 combines the encoded representations from multiple modalities into a unified format. This is typically achieved by projecting the modality-specific encoded data into a shared embedding space or through concatenation. The unified representation produced by the input projector enables the generative model to process and consider data across different modalities simultaneously. This integration is critical for tasks that involve cross-modal understanding or require the generative model to generate outputs that leverage information from diverse input sources. The unified representation serves as the input to the generative model backbone.

The generative backbone 350 is the core processing unit of the multimodal generative model. It is a large language model trained on extensive datasets, similar to generative models such as LLMs used for text-based tasks but adapted to handle multimodal inputs. The backbone takes the unified input representation from the input projector and generates outputs such as textual descriptions, summaries, and/or other creative or analytical content. By leveraging its ability to process multimodal data, the generative backbone 350 enables sophisticated reasoning and generation tasks that span multiple data types. The generative backbone 350 combines elements from different neural network designs to handle diverse data types effectively. For example, CNNs may be used for processing visual data, while transformer networks as described above may be used to handle textual data, enabling the model to extract and synthesize features from both images and text. This integration results in outputs that accurately represent the input data, reflecting a deep understanding of both modalities. The transformer architecture, known for its ability to manage sequential data, may be adapted to work alongside CNNs, allowing these models to benefit from the strengths of each neural network type.

The output projector 355 converts the processed data generated by the generative backbone 350 into the desired output format or modality. This transformation is used to produce outputs tailored to specific tasks or target applications. For example: in an image captioning task, the output projector 355 converts the model's representation into a sequence of words describing the image; and for tasks requiring outputs in multiple modalities, the output projector 355 can generate data in formats such as text, audio, or video. The output projector 355 ensures that the model's internal representations are effectively translated into actionable or interpretable outputs for end-users or downstream systems.

The modality generator 360 is an optional component designed for tasks that require the generation of outputs in specific modalities. It takes the outputs from the generative backbone 350 and generates data in the target modality, such as audio, video, or images. The modality generator 360 can be implemented using advanced generative techniques, including: Generative Adversarial Networks (GANs), Variational Autoencoders (VAEs), and Diffusion Models. For example, in a task requiring audio generation, the modality generator 360 transforms the model's output into synthesized audio. This component expands the capabilities of the multimodal generative model, allowing it to handle a wide range of tasks across diverse modalities.

In at least some instances, the self-attention mechanism, a cornerstone of transformer networks, is integral to the functioning of large multimodal models. It enables the model to weigh the importance of different elements within an input sequence, regardless of their position, allowing it to capture intricate relationships between various data types. For example, in an image captioning task, the model can associate specific visual features with corresponding descriptive text, enhancing the coherence and accuracy of the generated captions. By assigning scores to relationships between elements, the self-attention mechanism highlights the most relevant connections, enabling the model to focus on the most informative parts of the input data and perform complex multimodal tasks effectively.

In large multimodal models, data preprocessing is a step that ensures the input data is in a suitable format for the model to process. This involves tasks such as tokenization for text data, where the text is broken down into manageable pieces, and feature extraction for image data, where key visual elements are identified and encoded. By standardizing and normalizing different data types, preprocessing reduces the complexity of the input space, enabling the model to treat similar elements consistently. Effective preprocessing is important for the model to integrate information from various modalities and produce accurate, meaningful outputs.

Training large multimodal models involves optimizing their parameters through exposure to diverse datasets that include paired data from different modalities. This computationally intensive process often requires specialized hardware like GPUs or TPUs to manage the large volumes of data and the complexity of the model calculations. Techniques such as dropout and layer normalization are employed to improve model generalization and prevent overfitting. By iteratively adjusting the model's parameters, the training process enables the model to learn underlying patterns and relationships within the data, enhancing its ability to generate coherent and contextually relevant outputs across different modalities.

Evaluation and tuning of large multimodal models are conducted using various metrics tailored to the specific tasks they are designed to perform. For example, BLEU scores are used for text generation tasks, while accuracy is commonly applied for visual recognition tasks to assess performance. Tuning involves adjusting hyperparameters and refining training strategies based on evaluation results to enhance the model's effectiveness. This iterative process ensures that the model can perform a wide range of multimodal tasks with high accuracy and relevance, making it a versatile tool for applications requiring the integration of different types of data.

Large multimodal models represent a significant advancement in machine learning by leveraging sophisticated architectures that combine different neural network types and apply self-attention mechanisms. This enables them to perform complex tasks that require understanding and synthesizing information from diverse data types. Effective preprocessing, rigorous training, and thorough evaluation are crucial to their success, allowing these models to generate coherent and contextually relevant outputs across a wide range of applications.

In accordance with one or more embodiments, other types of models besides large language models and large multimodal models belong to the broad category of generative models. For example, stochastic models directly incorporate randomness into their structure, making them inherently generative as they can produce a diverse set of outputs for a given input. GANs learn to generate new data that is indistinguishable from the data they were trained on, using a dual-network architecture that involves a generative component. VAEs are explicitly designed for generating new data points by learning a distribution of the input data and encode inputs into a latent space and generate outputs by sampling from this space, making them inherently generative. Sequence-to-sequence models are generative in nature when used with sampling strategies. Although this list of generative model types is not exhaustive, it illustrates the broad use of the term generative model beyond large language models.

To generate a summary, the multimodal models 335 leverage the various components, models, and network architectures including semantic analyzer 365, segmentation generator 370, text processor 375, anomaly detector 380, and natural language processor 385 to identify various characteristics of the content 325. Instances when the content 325 includes audio or text, the multimodal models 335 can identify linguistic patterns, contextual cues, and structural elements from the content 325 to be summarized. Instances when the content 325 includes images, the multimodal models 335 can identify patterns like edges, textures, shapes, and colors within localized regions. The multimodal models 335 then apply its language or multimodal understanding capabilities to analyze the various characteristics of the content 325 for key themes, topics, and relationships between ideas or objects expressed or shown in the content 325. For example, for text, the multimodal models 335 may identify significant points, discern the tone of the conversation, and extracts critical information such as questions asked, decisions made, or actions agreed upon. For example, for an image, the multimodal models 335 may identify significant and/or insignificant objects, and organize extracted data to highlight the key findings, such as the presence or absence of certain medical conditions, potential diagnoses, or areas requiring further investigation. The multimodal models 335 focus on eliminating redundant or irrelevant content while retaining the essence of the content 325 such as the conversation or the image, ensuring that the summary is concise yet comprehensive.

The segmentation generator 370 generates segments of content 325 (e.g., text, audio, and images) by analyzing their structural or visual components and dividing them into meaningful units based on predefined criteria. For text and/or audio, a segmentation generator 370 identifies linguistic or semantic boundaries, such as sentences, paragraphs, or topics, using techniques like natural language processing (NLP), tokenization, and clustering. It may also detect patterns, such as keywords or stylistic shifts, to segment content into coherent thematic sections. For example, an NLP technique may include using a recursive text splitter to create segments of a particular size (e.g., a paragraph, a sentence, a word). For instance, a recursive text splitter may divide the content 325 into multiple paragraphs, where each paragraph is of a certain size (e.g., 500 tokens). If a paragraph is longer than 500 tokens, the text splitter may further break the paragraph into multiple chunks such that there is a chunk overlap of several (e.g., one or two) sentences between consecutive chunks. Another exemplary technique may include using speaker identification, where a segmentation generator 370 segments the text/audio based on who is speaking. The segmentation generator 370 can leverage speaker diarisation metadata (provided by an ASR, such as ASR system described in FIG. 2) or infer transitions through changes in tone or vocabulary to segment text/audio by speaker. Another exemplary technique may include using time-based segmentation, such as dividing a transcription or audio recording into 5-minute or 10-minute segments. This approach is useful for summarizing lengthy audio recordings, allowing the summary to reflect the progression of the discussion over time.

For images, another segmentation generator 370 can use computer vision techniques, such as edge detection, clustering, or deep learning models like Mask R-CNN or U-Net, to divide the image into regions or objects based on pixel-level features such as colors, textures, or shapes. Each segment is assigned a label or classification, such as “sky,” “building,” or “foreground object” for images, or “introduction,” “body,” and “conclusion” for text. These segmented units are then organized to facilitate further analysis, such as summarization, classification, or multimodal integration, enabling the segmentation generator 370 to extract and process relevant information from both text and visual data comprehensively.

Anomaly detector 380 can be used to determine the shapes, boundaries, and locations of the objects within image segments. These tools may utilize deep neural networks, including convolutional neural networks (CNNs), U-Nets, or vision transformers, to analyze an image at the pixel level. It detects patterns like edges, textures, shapes, and colors within localized regions using techniques such as thresholding, clustering, and edge detection. For pixel thresholding, methods like global thresholding and adaptive thresholding may be applied. In global thresholding, a single threshold is used for the entire image, classifying pixels into two categories based on whether their intensity is above or below a threshold. Adaptive thresholding applies multiple thresholds to account for variations in lighting, color saturation, and other factors. Pixels can then be clustered into predefined classes, such as background or target objects, based on their threshold classification. In some cases, pixels are further differentiated to distinguish between different instances within the same class, such as identifying pathological features in tumor tissue. Edge detection techniques may also be employed to identify areas of rapid pixel intensity changes, often by applying convolution kernels. Through successive layers of processing, Anomaly detector 380 can uncover increasingly complex features, such as objects, scenes, and spatial relationships.

Visual features detected by the anomaly detector 380 and text/audio segments generated from segmentation generator 370 can be input into embedding models to generate embedding vectors. The process of embedding involves transforming the visual features and text/audio segments into dense numerical vectors that capture the important information and semantical meanings of the segmented pieces. This allows the multimodal models 335 to process and understand the content 325 more effectively. The embedding models can include image embedding models, such as ResNet, VGG, CLIP, BLIP, etc. and text/audio embedding models such as BERT, GPT, VGGish, YAMNet, etc. The embedding models can pass the embeddings to one or more components, models or architectures, which are configured to understand relationships between text and/or visual and textual data. For example, the one or more components, models or architectures may have been trained on paired visual-text datasets (e.g., images with captions) to interpret the extracted visual features in the context of language. Another example may include one or more of the components, models or architectures having been trained on large amounts of text/audio data.

The multimodal models 335 can use natural language tools processor 385 to generate textual summaries for image content. The natural language tools processor 385 can use the embeddings generated by the modality encoder 340 and/or one or more embedding models to map the numerical representations of the embeddings to meaningful textual output. When the multimodal models 335 receive an embedding, they may use its pre-trained layers to decode the information and contextualize it within its vast knowledge base. The multimodal models 335 may optionally apply attention mechanisms to focus on relevant aspects of the embedding, ensuring coherence and accuracy in its interpretation.

To refine the summaries generated by the multimodal models 335, may utilize a text processor 375 to filter out filler words (e.g., “um,” “uh”), repetitions, interruptions, and other disfluencies in the content 325, to remove unnecessary noise for clearer segmentation. The text processor 375 may also add proper punctuation and structure to raw text from audio transcriptions or unformatted inputs. Another component, model, or architecture that may be used by the multimodal models 335 is the semantic analyzer 365 to help the model understand the meaning and context of the content 325 and prioritize important information. The semantic analyzer 365 may use named entity recognition to identify key entities (e.g., people, organizations, dates) in the text/recordings and ensures they are included in the summary, key word extraction to ensure the most relevant terms and phrases are included in the summary, and topic modeling to identify the primary topics or themes in the content 325 to guide the summarization process. Although not shown, the multimodal models 335 can include other components, models, or architectures such as summarization algorithms, metadata utilization tools, post-processors (e.g., grammar and spell check), and the like.

The summary generation system 315 generates a summary 320 for the content 325 using the multimodal models 335, unimodal models 330, or any combination thereof and outputs the summary 320 to a source (e.g., a requesting user) via the Ai-enabled subsystem 305 or other system/subsystem. In some instances, the summary 320 includes one or more layers to represent the original content 325. For example, the summary 320 may include a summary layer that only captures the content 325 to be summarized at a high level (e.g., single layer). In other examples, the summary 320 is a multi-layered summary. For example, in the context of multiple speakers, the summary 320 may include a first level that provides a summary of the perspective of each individual speaker, a second level that summarizes the combined or aggregated perspectives of the speakers (e.g., shared perspectives), a third and subsequent levels that summarize other aspects of the content 325.

The summary generation system 315 may use a variety of approaches to generate the summary 320 such as a descriptive summary approach, an analytical summary approach, and/or a task-specific summary approach. A descriptive summary approach may provide a high level or generalized overview of the content 325 to be summarized, whereas an analytical approach may explain the relationships and patterns in more detail. Further, a task-specific approach generates a summary focused on specific aspects relevant to the user's needs.

In some instances, for example during multi-speaker scenarios, the summary generation system 315 can combine text segmented by individual speakers, using the aggregator 390 to generate summary 320. For instance, as shown in Example 2 below, a multimodal model 335 receives content 325 that includes dialog from multiple speakers (e.g., Speaker_0 and Speaker_1). The segmentation generator 370 may use the technique of segmenting based on who is speaking (as described above) to initially generate individual summaries for each speaker. Then aggregator 390 can combine the individual summaries into a single summary representative of all speakers. In some instances, the aggregator 390 generates a consensus summary, highlighting the common ground reached by all speakers and integrates the key points on which the speakers agree into the summary.

### Example 2: Multi-Layered Summary from a MDT Discussion

```
{
  “case_summary”: {
    “″individual __ perspectives”: [
      {
        “doctor_name”: “Dr. Speaker_0”,
          “perspective_summary”: “Dr. Speaker_0 provided a detailed initial
          assessment of the 55-year-old female patient who presented with multiple
          episodes of lose stools and vomiting after consuming food from outside. The
          patient was conscious, oriented, and had stable signs. The primary survey
          indicated metabolic alkalosis with severe hypokalemia (potassium 2.1). An
          ECG showed U waves, ST depression, and T inversion. Dr. Speaker_0
          initiated potassium correction through IV and sent for routine
          investigations.”
      },
      {
        “doctor_name”: “Dr. Speaker_1”,
          “perspective_summary”: “Dr. Speaker_1 emphasized the importance of
          correcting severe hypokalemia and discussed the ECG changes associated
          with low potassium levels. Dr. Speaker_1 also highlighted the need to
          correct magnesium and phosphorus levels before potassium to ensure
          effective treatment. The patient was given potassium chloride syrup orally
          and IV potassium through a central line. Dr. Speaker_1 also discussed the
          potential causes of hypokalemia and the importance of monitoring and
          correcting electrolyte imbalances.”
      },
    ],
    “combined_perspective”: {
      “summary”: “The patient, a 55-year-old female, presented with multiple episodes of
loose stools and vomiting after consuming food from outside. Initial assessment showed
metabolic alkalosis with severe hypokalemia. ECG changes confirmed the low potassium
levels. The patient was treated with IV and oral potassium, along with magnesium and
phosphorus correction. The patient also received IV fluids and antibiotics for suspected
gastroenteritis. Over the course of the hospital stay, the patient's electrolyte levels normalized,
and her symptoms improved.”,
      “consensus_recommendation”: “The consensus recommendation is to correct the
electrolyte imbalances, particularly potassium, magnesium, and phosphorus, while monitoring
the patient's cardiac status. Additionally, treat the underlying gastroenteritis with appropriate
antibiotics and provide supportive care with IV fluids.”
        },
      “unbiased_recommendation”: {
        “summary”: “Based on the combined perspectives and findings, the unbiased
recommendation for the patient is to continue monitoring and correcting electrolyte
imbalances, particularly potassium, magnesium, and phosphorus. Ensure cardiac monitoring
during potassium correction. Treat the underlying cause of the symptoms, which is likely
gastroenteritis, with appropriate antibiotics and supportive care. Reassess the patient's
condition regularly to adjust treatment as needed.
      },
    “next_steps”: {
      “summary”: “The recommended next steps include electrolyte correction, particularly
potassium, magnesium, and phosphorus, with both IV and oral supplementation. Monitor the
patient's cardiac status closely during potassium correction. Administer antibiotics to treat
underlying gastroenteritis and provide supportive IV fluids. Reassess the patient's electrolyte
levels and overall condition regularly to adjust treatment as needed.”
      },
    “follow_up_plan”:
      “summary”: “The follow-up plan includes regular follow-up appointments to monitor
the patient's electrolyte levels and overall health. Ensure the patient understands the
importance of maintaining a balanced diet and staying hydrated. Educate the patient on
recognizing symptoms of electrolyte imbalances and when to seek medical attention. Plan for
periodic reassessment of patient's condition to ensure long-term stability and prevent
recurrence of symptoms.”
      }
    }
}
```

In some instances, at least one of the models (e.g., multimodal model 335) used by the summary generation system 315 is a multimodal LLM. As discussed herein, multimodal LLMs are advanced AI systems designed to process and integrate multiple types of data, such as text, images, audio, and video, within a unified framework. Unlike traditional LLMs that focus solely on text, multimodal LLMs leverage specialized components like vision transformers for images, spectrogram-based models for audio, and contextual embeddings for text to understand and align diverse modalities. This model is trained on datasets containing paired inputs (e.g., images with captions or audio with transcripts) to establish relationships across different types of data. By combining their expertise in natural language processing with computer vision and other sensory modalities, multimodal LLMs can generate coherent responses tailored to specific tasks, such as describing images, answering audio-based queries, or creating captions for videos. For example, during an MDT discussion, the multimodal LLM can analyze medical images, interpret textual descriptions of a patient and/or electronic health records, and use audio dialog from healthcare providers, to develop a comprehensive treatment plan. As such, a multimodal LLM may access additional information from repository 310 to integrate into the summary 320. The repository may store electronic health records, clinical data, patient records, medical labs, treatment plans, medications, etc. that the summary generation model 315 can use to validate its image summary report. Example 3, provided below, shows an exemplary summary output of the radiology images shown in FIG. 4:

### Example 3: Image Summary from a MDT Discussion

```
{
  “summary”: “The CT images show multiple areas of lung involvement with varying
degrees of consolidation, ground-glass opacities, and possible fibrotic changes. These findings
are suggestive of a severe pulmonary condition, potentially indicative of an infectious or
inflammatory process.”,
“insights_and_anomolies”: {
  “image_a”: {
    “description”: “The image shows bilateral ground-glass opacities with some areas of
consolidation, particularly in the lower lobes.”,
    “anomalies”: [“Bilateral ground-glass opacities”, “Areas of consolidation”]
  },
  “image_b”: {
    “description”: “This image reveals patch ground-glass opacities with some interstitial
thickening, more prominent in the right lung.”,
    “anomalies”: [“Patchy ground-glass opacities”, “Interstitial thickening”]
  },
  “image_c”: {
    “description”: “There is excessive bilateral consolidation with ground-glass opacities
and possible fibrotic changes, indicating a more advanced stage of disease.”,
    “anomalies”: [“Extensive bilateral consolidation”, “Ground-glass opacities”, “Possible
fibrotic changes”]
    },
    “image_d”: {
      “description”: “The image shows bilateral ground-glass opacities with some areas of
consolidation and interstitial thickening, similar to the findings in image b but more
widespread.”,
      “anomalies”: [“Bilateral ground-glass opacities”, “Areas of consolidation”, “Interstitial
thickening”]
    }
}
    “UnbiasedConclusion”: “The CT images suggest a sever pulmonary condition
characterized by bilateral ground-glass opacities, areas of consolidation, and possible fibrotic
changes. These findings are consistent with a sever infectious or inflammatory process, such
as COVID-19 pneumonia or another form of interstitial lung disease.”,
    “treatment_plan”: {
      “initial_steps”: [
        “Administer supplemental oxygen to maintain adequate oxygen saturation.”,
        “Initiate broad-spectrum antibiotics to cover potential bacterial superinfection.”,
        “Consider antiviral therapy if a viral etiology is suspected.”,
        “Administer corticosteroids to reduce inflammation if indicated.”
      ],
      “further_management”: [
        “Monitor respiratory status closely with regular follow-up imaging.”,
        “Evaluate the need for mechanical ventilation if respiratory failure ensures.”,
        “Consult a pulmonologist for further evaluation and management.”,
        “Consider antibiotic therapy if fibrotic changes are confirmed.”
    ],
      “preventive_measures”: [
        “Encourage vaccination against respiratory pathogens.”,
        “Promote goo hand hygiene and use of personal protective equipment.”,
        “Advise avoiding exposure to known respiratory irritants.”,
        “Recommend regular follow-up with a healthcare provider for early detection of any
change.”
    ],
  },
    “UserQueryResponse”: ‘’
}’’’
```

The summaries illustrated above are merely examples of summaries generated by the summary generation system 315 and are not intended to unduly limit the scope of claimed embodiments. One of ordinary skill in the art would recognize many possible variations, alternatives, and modifications. For example, in some implementations, the system may generate different summaries for the same content based upon changes to the configuration of the summary generation system 315, the types of prompts provided to the models, the summarization strategy used (e.g., chain of thought prompting technique, a multi-hop question answer evaluation) by the models, the input parameters (setup) for the model and the like. In addition, while the examples illustrated above are examples of an MDT discussion summary that is generated by the summary generation system 315, in alternate embodiments, the summary generation system 315 may include capabilities to generate summaries for various other types of content such as such as documents, webpages, news articles, research papers, and so on.

### Multi-Disciplinary Platform

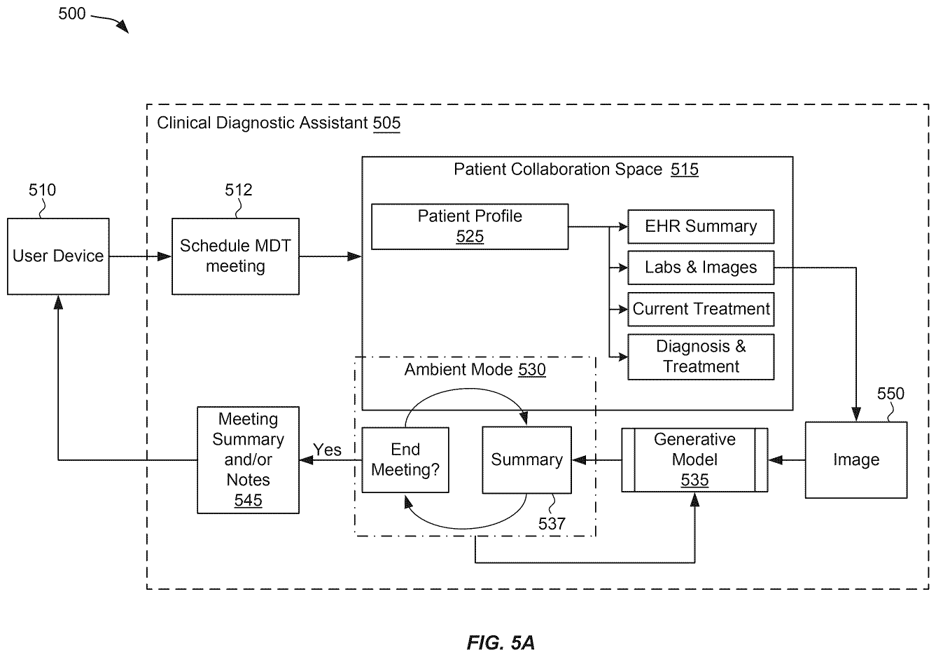

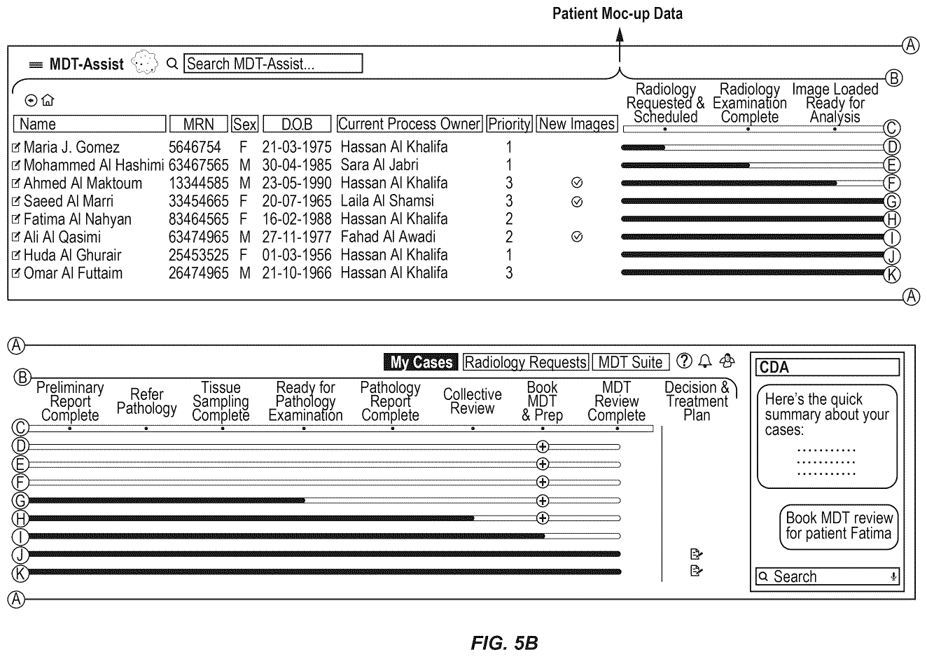

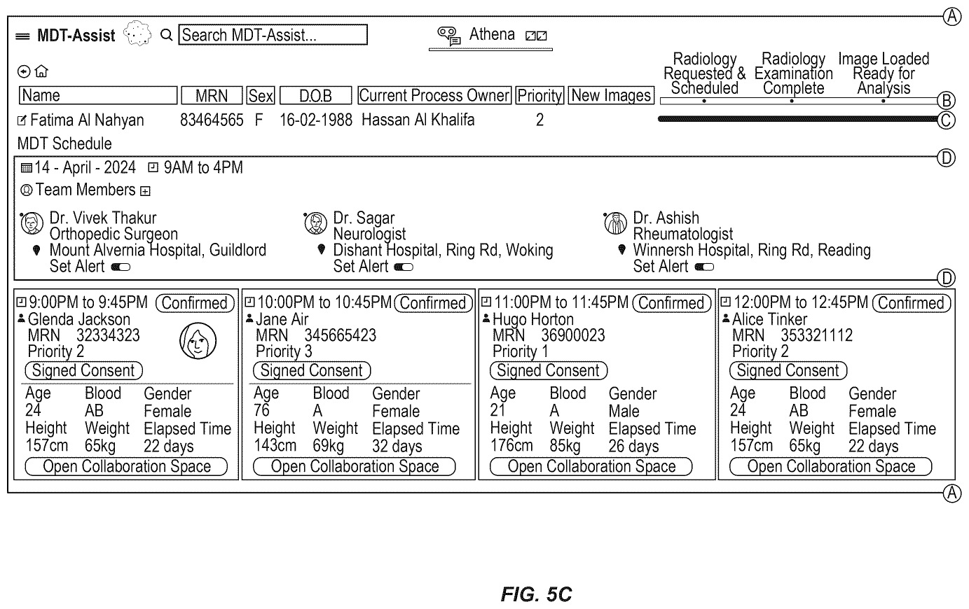

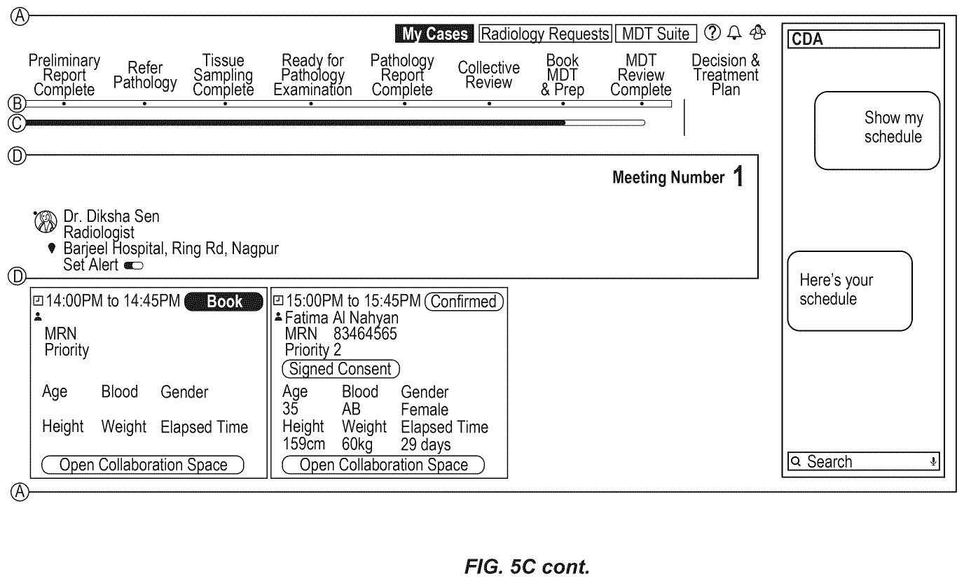

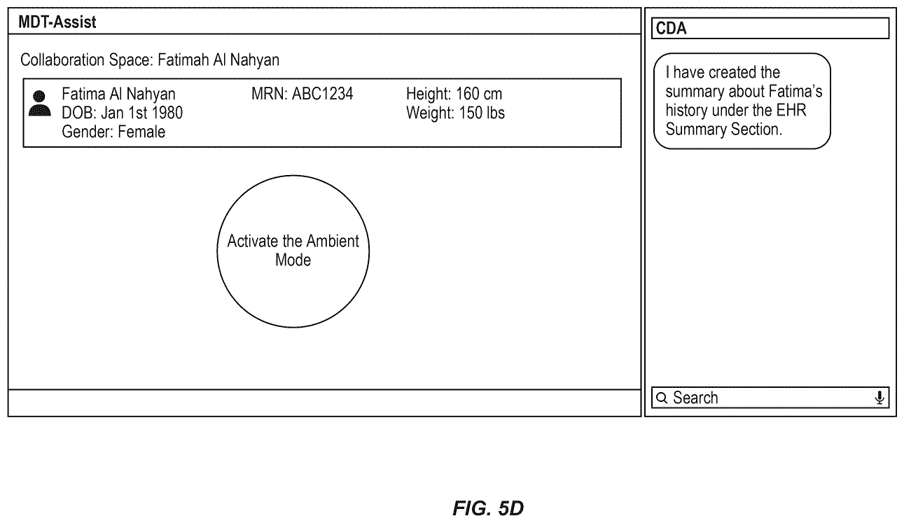

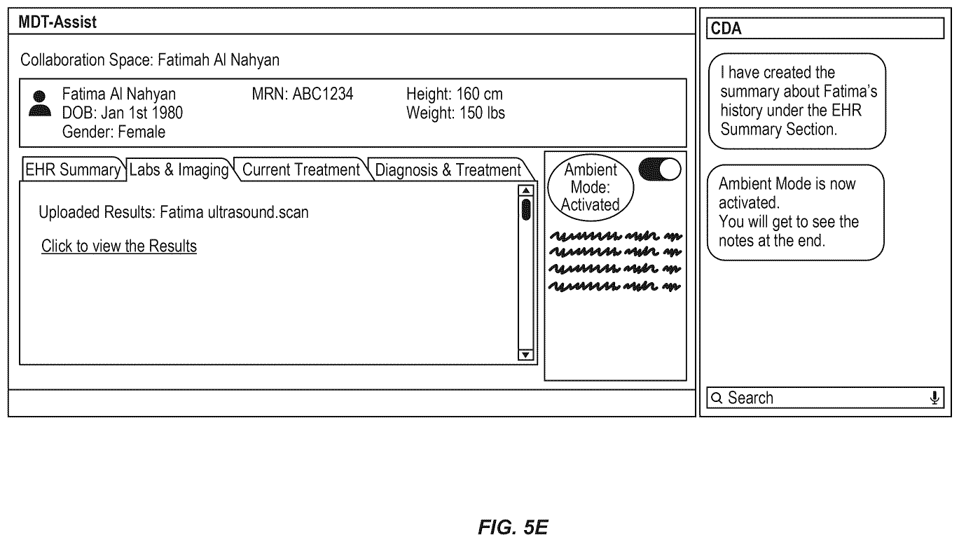

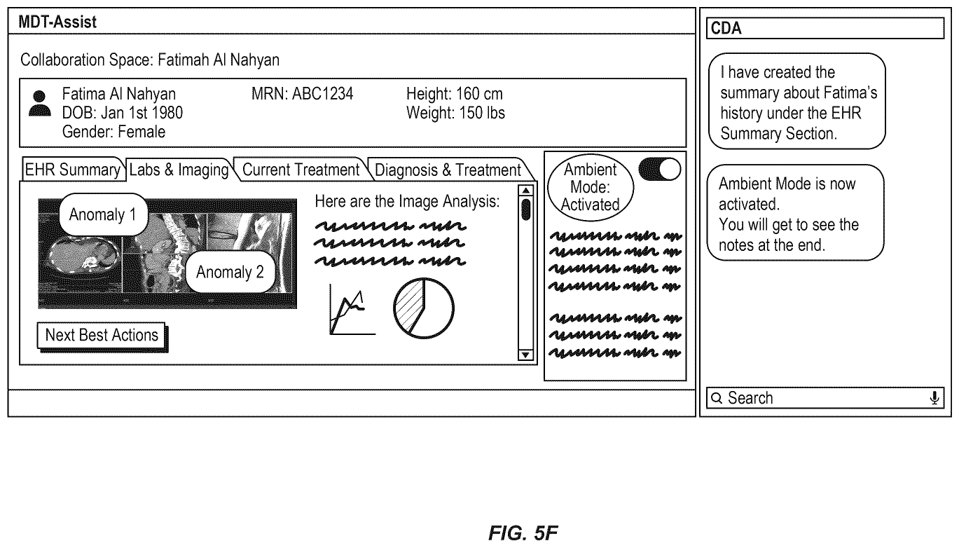

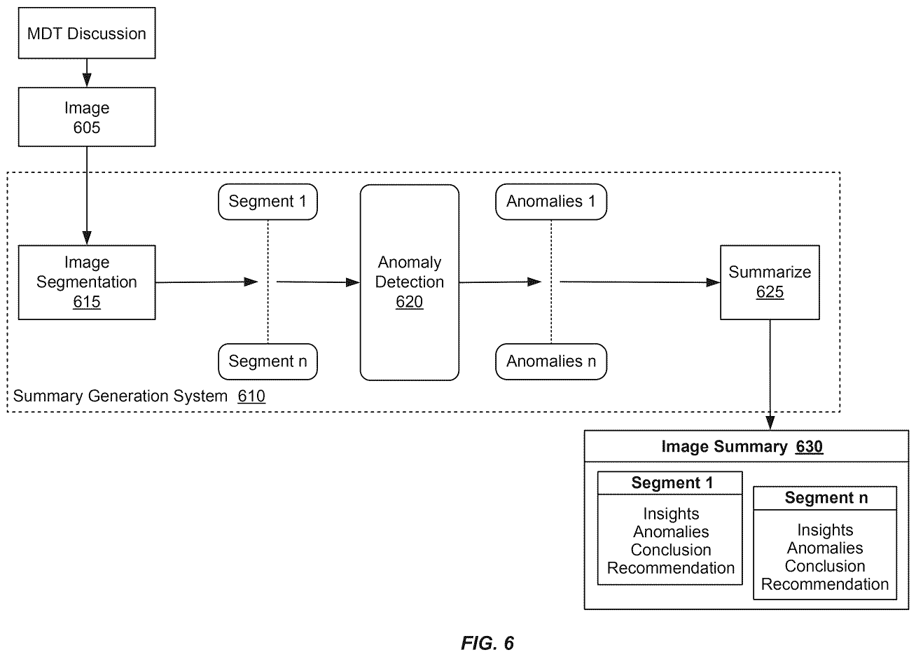

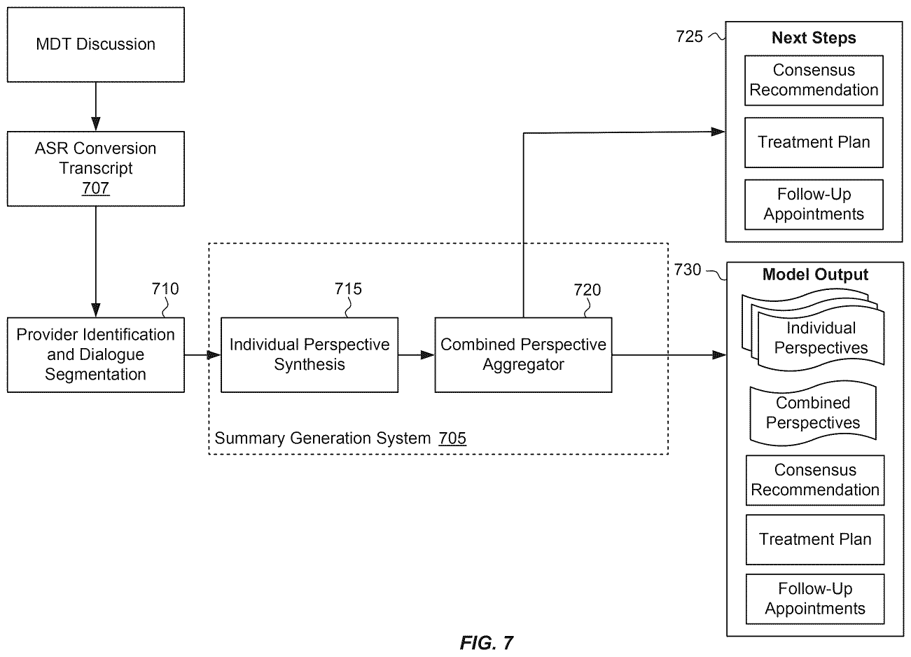

FIG. 5A is a block diagram summarizing an exemplary workflow 500 for using a Clinical AI Agent having an MDT-Assist component (e.g., plugin for a clinical digital assistant application) to facilitate review of patient health care by a MDT of healthcare providers, according to various embodiments. The workflow depicted in FIG. 5A may be implemented in software (e.g., code, instructions, a program) executed by one or more processing units (e.g., one or more processors, cores) of the respective systems, hardware, or combinations thereof described with respect to FIGS. 1-4 and 9-13). The software may be stored on a non-transitory storage medium (e.g., on a memory device). Although the processes presented in FIG. 5A depict the various processing steps occurring in a particular sequence or order, this is not intended to be limiting. In certain alternative embodiments, the steps may be performed in parallel and/or in a different order. In certain embodiments, such as in the embodiments depicted in FIGS. 1-3, the workflow depicted in FIG. 5A may be performed by system/computing environment 100, 200, and/or 300, as described further with respect to FIGS. 6A and 7A.

Clinical environments, such as healthcare facilities, increasingly rely on AI-powered assistants or agents such as the Clinical AI Agent to streamline patient care and record-keeping processes. FIG. 5A illustrates one approach for how a Clinical Diagnostic Assistant (CDA) (also referred to herein as the Clinical AI Agent) 505 having an MDT-Assist component may be used by a multidisciplinary team of healthcare professionals for review of patient health care and the generation of a comprehensive summary of the review (e.g., a summary of the MDT meeting discussion and/or a summary of one or more medical images reviewed in the MDT meeting). Initially, healthcare providers log into the CDA 505 having an MDT-Assist component using their respective user devices 510 (client devices 110 described with respect to FIG. 1), to access a dashboard in a graphical user interface (GUI) displaying their assigned patient cases (see FIG. 5B). For each patient case on the dashboard, CDA 505 provides medical data including information related to patient identifications (e.g., name, sex, date of birth, etc.), availability of new records (e.g., medical images, lab results, etc.), and whether certain tasks have been completed. Examples of tasks, in the non-limiting context of radiology, may include radiology request and scheduling, radiology examination complete, images loaded and available for analysis, preliminary reports, pathology referral, tissue sample collection complete, pathology examination complete, pathology report complete, collective review, schedule and participation in a MDT review meeting, MDT review complete, decision and treatment plans, and the like.

The healthcare provider uses the dashboard to select a patient case that they desire to review. Once a patient case is selected, the healthcare provider, in this example the radiologist, can schedule an MDT review meeting with the assistance of the CDA 505 (MDT-Assist plug-in or module 512) (see FIG. 5C). The CDA 505 may check the availability of the other healthcare providers, for example, in required specializations, and facilitates scheduling for in-person or virtual meetings through meeting platforms like Teams, Zoom, or 8×8. Additionally, a healthcare provider may request to view their schedule of meetings, cases, appointments, etc. on the dashboard. The hosting healthcare provider then selects a suitable time slot for an in-person or virtual meeting. At the scheduled time, the MDT review meeting begins.

When an MDT review meeting starts, CDA 505 opens the patient collaboration space 515 (see FIG. 5D). In the patient collaboration space 515, CDA 505 can be used to look up the focus of the MDT review meeting (e.g., a patient) and display, for example, a corresponding patient profile 525 enabling healthcare providers to access and search for information in electronic health records (e.g., medical history, family history, notes, prior summaries, medications, current treatments, lab or imaging reports, and the like) corresponding to the patient. The CDA 505 with its context capabilities can also display any summaries generated for or requested to be generated for the patient within the collaboration space 515 (see FIG. 5E). The CDA 505 via the collaboration space 515 enables the healthcare providers to navigate within the patient collaboration space 515 by audio request or physically selecting various user interface tabs included in the patient profile 525. For example, during the meeting, the healthcare providers can ask CDA 505 to show patient details like medical history, last notes, medications, current treatment, and new lab or imaging reports.

During the MDT review meeting, the CDA's ambient mode 530 may be activated manually (e.g., via a healthcare provider audio request or by physically clicking on a user interface element) or automatically (e.g., upon opening the patient collaboration space 515 or a patient report in the patient collaboration space 515) to record and transcribe the conversations among the healthcare providers during the MDT review meeting. While in ambient mode, CDA 505 may automatically identify participants in the MDT review meeting and generate a conversation transcript using automatic speech recognition technology as described in detail with respect to FIG. 2. In some instances, the activation of the CDA's ambient mode 530 automatically triggers execution of a summary process for the conversations among the healthcare providers during the MDT review meeting. In other instances, while in ambient mode 530, a healthcare provider in the MDT review meeting can manually trigger via audio request or physical means (e.g., clicking on a user interface element) execution of a summary process for the conversations among the healthcare providers during the MDT review meeting. The summary process for the conversations among the healthcare providers is performed by one or more generative models 535 as described in detail with respect to FIGS. 3 and 7A. The result of the summary process is one or more summaries 537 of the conversations among the healthcare providers during the MDT review meeting. The one or more summaries can then be communicated at the end of the MDT review meeting as raw summaries or summaries with annotations/notes 545 to the user devices 510 associated with the healthcare providers. In some instances, the summaries may be stored in one or more repositories (e.g., databases 122 as described with respect to FIG. 1) for subsequent retrieval (e.g., as part of the patient profile 525).

During the MDT review meeting, a healthcare provider via the CDA 505 may navigate in the patient collaboration space 515 to a corresponding tab (e.g., a labs and reports tab in the patient profile 525) to access one or more images 550 (e.g., medical images) for discussion during the MDT review meeting. In some instances, accessing the one or more images automatically triggers execution of an analysis and summary process for the one or more images in real time during the MDT review meeting. In other instances, a healthcare provider in the MDT review meeting can manually trigger via audio request or physical means (e.g., clicking on a user interface element) execution of the analysis and summary process for the one or more images in real time during the MDT review meeting. For example, the healthcare provider can ask the CDA 505 to show insights related to the one or more images 550 or click on an AI medical button next to the imaging report or one or more images 550 for detailed insights. The analysis and summary process for the one or more images 550 among the healthcare providers is performed by one or more generative models 535, as described in detail with respect to FIGS. 3 and 6A. The result of the analysis and summary process is one or more summaries 537 of the analysis of the one or more images. The one or more summaries can then be communicated at the end of the MDT meeting or during the meeting as raw summaries or summaries with annotations/notes 545 to the user devices 510 associated with the healthcare providers (see FIG. 5F). In instances of a virtual meeting, the CDA 505 may allow the panel of healthcare providers in the MDT review meeting to perform their own annotations over the report and one or more summaries 537. In some instances, the summaries may be stored in one or more repositories (e.g., databases 122 as described with respect to FIG. 1) for subsequent retrieval (e.g., as part of the patient profile 525).

As shown in FIG. 6, in some instances, during an MDT review meeting there may be a desire by a healthcare provider to review and analyze one or more images 605 (e.g., images 550 described with respect to FIG. 5A). As such, a healthcare provider via the CDA may navigate in the patient collaboration space to a corresponding tab (e.g., a labs and reports tab in the patient profile) to access one or more images 605 for discussion during the MDT meeting. In some instances, the one or more images 605 are medical images. Examples of medical images include, without limitation, X-rays, CTs, MRIs, ultrasounds, PET scans, fluoroscopy images, mammograms, endoscopy or internal imaging, dermatological images, ophthalmic imaging, dental imaging, bone densitometry imaging, histopathology imaging, and any other imaging results that the patient may have.

As described above, in some instances, accessing the one or more images 605 automatically triggers execution of an analysis and summary process for the one or more images in real time during the MDT review meeting. In other instances, a healthcare provider in the MDT meeting can manually trigger via audio request or by physical means (e.g., clicking on a user interface element) execution of the analysis and summary process for the one or more images 605 in real time during the MDT review meeting. The analysis and summary process for the one or more images among the healthcare providers is performed by one or more generative models of the summary generation system 610 (e.g., the summary generation system 315 described with respect to FIG. 3). The summary generation system 610 uses one or more generative models comprising various components, models, and networks, such as an image segmentation generator 615 (e.g., segmentation generator 370 described with respect to FIG. 3), anomaly detector 620 (e.g., anomaly detector 380 described with respect to FIG. 3), and a summarization generator 625 (e.g., generative backbone 350 including natural language processor 375 and text processor 375 described with respect to FIG. 3), to provide a detailed, annotated summary of the image(s) being viewed.

The one or more generative models are capable of performing real-time segmentation of the one or more images 605. Using advanced computer vision techniques integrated with the generative model's multi-modal capabilities, the model identifies and highlights distinct anatomical structures and regions of interest within the image. This segmentation allows for a focused analysis of specific areas, such as tumors, lesions, or other abnormalities, reducing the cognitive load on radiologists and enabling faster identification of critical findings. More specifically, the image segmentation generator 615 leverages computer vision techniques, such as edge detection, clustering, or deep learning models to divide the image into regions or objects based on pixel-level features within the one or more images 605. Features can include organs, tissues, tumors, blood vessels, cells, and the like. The image segmentation generator 615 can utilize several different approaches to generate image segments including manual segmentation, semi-automatic segmentation, and/or automatic segmentation. In manual segmentation, a trained healthcare provider (e.g., a radiologist or other medical professional) can manually identify features using specialized software. In semi-automatic and automatic segmentation techniques, image segments are generated with minimal or no intervention from healthcare providers, respectively. Additionally, image segmentation tool 615 can automatically apply one or more techniques for image segmentation such as semantic, instance, or panoptic segmentation. For example, a thresholding technique may be applied, for example to X-ray or high contrast images, where pixel values are divided into foreground and background based on intensity levels. In some cases, a region-based segmentation technique may be used, for example on MRIs to segment tumors, to group pixels into regions based on similarity in intensity, texture, or color. Other clustering techniques may also be used, such as k-means clustering or fuzzy C-means clustering. In some cases, an edge-based segmentation technique may be used, for example to detect organ boundaries in CT scans, to detect edges or boundaries between regions based on changes in intensity or gradients. In some cases, segmentation techniques that remove noise or fill in gaps (e.g., morphological operations, Gaussian filters, or median filters) may be used. In some cases, machine learning-based segmentation techniques may be used, for example to identify tumor segments in CTs or MRIs, such as support vector machines (SVMs), random forest, deep learning-based models (e.g., CNNS, U-nets, fully convoluted networks, mask R-CNNs, and the like). The image segmentation tool 615 outputs segments for the one or more images 605 (segments 1-n as shown in FIG. 6).

Once the one or more images 605 are segmented, the one or more generative models use the anomaly detector 620 to automatically analyze each segment to detect anomalies, such as unusual growths, structural deformities, or signs of disease. The one or more generative models then generate detailed insights for each identified anomaly, providing context and possible implications based on the patient's clinical history and similar cases from its extensive knowledge base. These insights are presented in a clear and concise manner, facilitating easy interpretation by all MDT healthcare providers, regardless of their specialty. More specifically, the anomaly detector 620 can leverage deep neural networks (DNNs), including convolutional neural networks (CNNs), U-Nets, or vision transformers, to analyze the segments output from the image segmentation generator 615 at the pixel level. Using DNNs, anomaly detector 620 analyzes the segments by learning hierarchical patterns and features within the segments to detect anomalies. Convolutional layers are applied to the segments to extract low-level features such as edges and textures, followed by deeper layers that capture more complex and abstract patterns, such as shapes and structures indicative of abnormalities. Through training on annotated datasets, the network learns to associate these features with specific anomalies, such as tumors, fractures, or lesions. Advanced models like U-Net or Mask R-CNN specialize in pixel-level segmentation, enabling precise isolation of abnormal regions. By leveraging activation maps and probability scores, the network highlights potential anomalies and classifies them based on learned patterns. This automated and data-driven approach allows DNNs to detect subtle and complex anomalies with high accuracy, aiding in faster and more reliable diagnostic decision-making. The anomaly detector 620 is capable of detecting a wide range of anomalies across various body systems from the one or more images 605.

For example, in the skeletal system, fractures, dislocations, osteoporosis, and bone tumors can be identified from X-rays, CT scans, or MRIs. In chest imaging, X-rays and CT scans can reveal lung conditions like pneumonia, pneumothorax, pulmonary embolism, or heart abnormalities like cardiomegaly and aortic dissection. In abdominal imaging, anomalies like kidney stones, liver tumors, appendicitis, and intestinal obstructions can be identified. In neurological imaging, MRIs and CT scans can identify strokes, brain tumors, aneurysms, and multiple sclerosis. In the cardiovascular system, CT angiography, echocardiograms, or MRIs can detect anomalies, such as coronary artery disease, aneurysms, and valve disorders. In breast imaging, mammograms and ultrasounds can identify anomalies like breast cancer, cysts, and fibroadenomas. In the reproductive system, imaging detects ovarian cysts, uterine fibroids, and ectopic pregnancies. In vascular imaging, anomalies such as stenosis, varicose veins, and arteriovenous malformations can be identified. In soft tissue imaging, anomalies like abscesses, lipomas, and sarcomas, may be detected. In oncology-related imaging, anomalies such as primary tumors, metastases, and lymphadenopathy can be identified. In infectious disease imaging, anomalies like tuberculosis, osteomyelitis, and abscesses can be identified.

Following anomaly detection, the anomalies are output (anomalies 1-n) and the summarization generator 625 generates a detailed, annotated summary of the one or more images 605 being viewed (see Example 3 above). To convert image analysis into text, the summarization generator 625 leverages the ability of the one or more generative models to process vast amounts of data and recognize patterns across numerous cases to generate textual summaries for the one or more images 605 being viewed. The summarization generator 625 can also integrate clinical data and patient history available in the patient collaboration space to generate a comprehensive image summary 630. In some instances, as shown in FIG. 6, the image summary 630 may include a summary for each of the segments processed by the anomaly detector 620. The image summary 630 can include insights, a list of the anomalies detected, a consensus conclusion of the multidisciplinary team, and a recommended treatment plan for the patient based on each segment. The image summary 630 can then be communicated at the end of the MDT meeting or during the meeting as a raw summary or a summary with annotations/notes to the user devices associated with the healthcare providers. In instances of a virtual meeting, the CDA may allow the panel of healthcare providers in the MDT review meeting to perform their own annotations over the report and the image summary 630. In some instances, the image summary 630 may be stored in one or more repositories (e.g., databases 122 as described with respect to FIG. 1) for subsequent retrieval.

As shown in FIG. 7, in some instances, while in ambient mode, the CDA leverages automatic speech recognition technology to generate one or more summaries of the conversation between the health care providers in the MDT review meeting. FIG. 7 illustrates how the CDA participates in dialogs or conversations between healthcare providers during the MDT review meeting, using one or more generative models of the summary generation system 705 (e.g., the summary generation system 315 described with respect to FIG. 3). The summary generation system 705 first generates individual sections from the text transcript 707 that summarize the viewpoint of each healthcare provider involved in the patient's case. This ensures that each healthcare provider's insights and recommendations are clearly articulated and preserved, allowing for a comprehensive understanding of the case from multiple angles. For example, the CDA may include an ASR model/system (e.g., see ASR computing system 202 with respect to FIG. 2) that generates the text transcript 707 from the audio of the healthcare providers during the MDT review meeting. The ASR generates speaker diarisation metadata that is used to identify each provider and generate the dialogue segmentations 710. The summary generation system 705 utilizes one or more generative models to synthesize individual perspective viewpoints 715 for each of one or more of the healthcare providers in the MDT review meeting.

After summary generation system 705 outlines each perspective, the summary generation system 705 synthesizes the information using the aggregator to produce a consensus summary 720. This section of the summary highlights the common ground reached during the discussion, integrating the key points where the specialists agree. The consensus summary 720 serves as a unified narrative that aligns the healthcare provers' collective understanding of the patient's condition and potential treatment approaches. The summary generation system 705 may then be used to formulate consensus recommendation, a treatment plan, and/or follow-up appointments by evaluating the intersection of the various perspectives. By focusing only on the overlapping recommendations, the summary generation system 705 mitigates individual biases and ensures that the treatment plan is balanced and reflective of the collective expertise. This approach not only captures the most agreed-upon strategies but also provides a clear and actionable plan that is less likely to be swayed by any single viewpoint. The result is a structured, multi-layered summary that offers a thorough and unbiased view of the patient's case, ensuring that all relevant perspectives are considered, and that the treatment plan is both comprehensive and well-grounded in the collective expertise of the MDT. This solution streamlines the summarization process, reduces the cognitive load on team members, and facilitates more efficient and effective decision-making.

As shown in FIGS. 7A and Example 2 above, the multi-layered structure summary includes the next steps 725 and model outputs 730. The next steps 725 can include the consensus recommendation based on the combined perspectives of the individual healthcare providers. In some instances, the next step 725 can further include the treatment plan based on the combined perspectives of the individual healthcare providers. In some instances, the next step 725 can further include suggested follow-up appointments and/or schedule follow-up appointments for the patient based on the discussion and/or treatment plan. The model outputs 730 can include the individual perspectives of each healthcare provider in the MDT review meeting, the aggregated combined perspectives of the healthcare providers, the consensus recommendation, the treatment plant, and/or the follow-up appointments for the patient. In some instances, the summary generation system 705 additionally generates any compliance issues, e.g., if a biopsy should be done within 24 hours instead of 10 days as per regulations in the meeting summary notes. The meeting summary notes may be composed and output as part of the multi-layered structure summary that includes the individual perspectives of each healthcare provider in the first layer, the combined perspectives in the second layer, and the next steps and model outputs (i.e., 725 and 730 with respect to FIG. 7). In some instances, more or fewer layers may be included in the meeting summary notes, depending on the patient profile discussed. The next steps 725 and/or model outputs 730 can then be communicated at the end of the MDT meeting or during the meeting as a raw summary or a summary with annotations/notes to the user devices associated with the healthcare providers. In instances of a virtual meeting, the CDA may allow the panel of healthcare providers in the MDT review meeting to perform their own annotations over the next steps 725 and/or model outputs 730. In some instances, the next steps 725 and/or model outputs 730 may be stored in one or more repositories (e.g., databases 122 as described with respect to FIG. 1) for subsequent retrieval.

### Illustrative Method

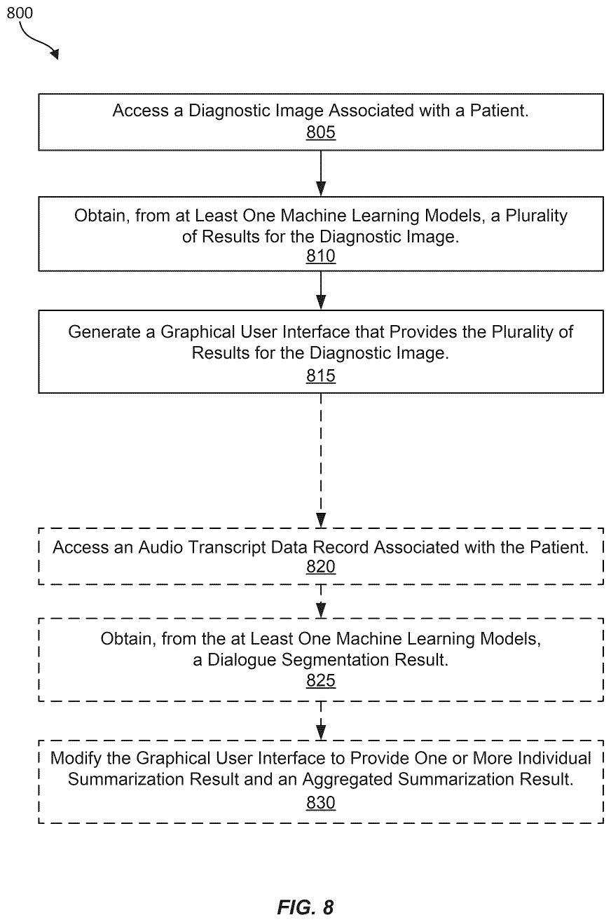

FIG. 8 is a flowchart illustrating a process 800 for use of multimodal generative models to generate concise and informative summaries of cross-modal inputs associated with MDT, according to various embodiments. The processing depicted in FIG. 8 may be implemented in software (e.g., code, instructions, a program) executed by one or more processing units (e.g., one or more processors, cores) of the respective systems, hardware, or combinations thereof described throughout. The software may be stored on a non-transitory storage medium (e.g., on a memory device). Although the methods presented in FIG. 8 depict the various processing steps occurring in a particular sequence or order, this is not intended to be limiting. In certain alternative embodiments, the steps may be performed in parallel and/or in a different order. In certain embodiments, such as in the embodiments depicted in FIGS. 1-3 and FIGS. 5-7A, the processing depicted in FIG. 8 may be performed by the environments and systems 100, 200, 300, 500, 600, and/or 700, as described with respect to FIGS. 1, 2, 3, 5, 6A, and 7A.

At step 805, a medical image associated with a patient is accessed. In some implementations, more than one medical image (e.g., 2, 3, 4, or more medical images) are accessed. The medical image(s) are medical images such as X-rays, CTs, MRIs, ultrasounds, PET scans, fluoroscopy images, mammograms, endoscopy or internal imaging, dermatological images, ophthalmic imaging, dental imaging, bone densitometry imaging, histopathology imaging, and any other imaging results that the patient may have. In some instances, the medical images are radiology images, such as X-rays, CT scans, MRIs, and the like. In some implementations, the medical image(s) is stored in a cloud service provider platform such as the platform 114 described above and/or in a database such as the databases 122 described above. In some implementations, the medical image(s) is stored in the platform and/or the database in association with an electronic record or electronic records of a healthcare provider and/or in association with and/or within an electronic health record or electronic health records of the patient of the healthcare provider. In some implementations, the medical image(s) is accessed from the platform and/or the database by a CDA, such as CDA 505 described with respect to FIG. 5A, where the CDA includes an application (e.g., CDA app with MDT-Assist) installed on, executing on, and/or accessed by one or more client devices.

At step 810, a plurality of results for the medical image(s) are obtained from at least one machine learning model based on a plurality of machine learning model prompts. The plurality of results comprise an image segmentation result for the medical image(s), an anomaly detection result for the medical image(s), and an image analysis summarization result for the medical image(s). To obtain the image segmentation results for the medical image(s), one or more first machine learning model prompts are used. In some implementations, at least one first machine learning model prompt of the one or more first machine learning model prompts comprises the medical image. A particular machine learning model prompt included in the one or more first machine learning model prompts is generated, based at least on the medical image(s). As described herein, prompt generation is the process of designing input instructions for machine learning models that clearly define the task, context, and desired output to guide the model's behavior effectively. A well-constructed prompt typically includes concise language, relevant context, and actionable goals, ensuring that the model can interpret the input accurately and generate meaningful responses. Additionally, prompt engineering can include the use of examples, formatting cues, or constraints to further refine the output and align it with user expectations. Once generated, the one or more first machine learning model prompts are provided to the at least one machine learning model. The image segmentation result(s) are received from the at least one machine learning model, where the image segmentation result(s) include image segment data representing structural variations (e.g., structural tissue variations) depicted in the medical image.

To obtain the anomaly detection results for the medical image(s) from the at least one machine learning model, a one or more second machine learning model prompt is used. A particular machine learning model prompt included in the one or more second machine learning model prompts is generated, based at least on the image segmentation data included in the image segmentation result. At least one second machine learning model prompt of the one or more second machine learning model prompts comprise the image segmentation result. Once generated, the one or more second machine learning model prompts are provided to the at least one machine learning model. The anomaly detection result(s) are received from the at least one machine learning model, where the anomaly detection result(s) includes a) one or more anomaly labels associated with a respective image segment in the image segment data, and b) text data describing a respective context associated with each of the one or more anomaly labels, the respective context based on a combination of each of the anomaly labels and electronic health data that is associated with the patient. In some implementations, based on the anomaly detection result(s), a CDA asks the user to identify one or more anomalies that are the primary focus for the anomaly detection result.

To obtain the image analysis summarization result for the medical image(s) from the at least one machine learning model, one or more third machine learning model prompts are used. A particular machine learning model prompt included in the one or more third machine learning model prompts is generated, based at least on the one or more anomaly labels included in the anomaly detection result. At least one third machine learning model prompt of the one or more third machine learning model prompts comprise the anomaly detection result. Once generated, the one or more third machine learning model prompts are provided to the at least one machine learning model. The image analysis summarization result is received from the at least one machine learning model, where the image analysis summarization result includes treatment recommendation data associated with the one or more anomaly labels. The treatment recommendation data describes a treatment option for a structural variation (e.g., a structural tissue variation) represented by the respective image segment.

In various embodiments, the plurality of results for the medical image(s) are obtained from a summary generation system that utilizes at least one generation models to generate one or more summaries for input data (e.g., text, images, audio) as described with respect to FIG. 3. The at least one generation models comprise LMMs and/or LLMs. As described above, the large multimodal model(s) integrate specialized neural network architectures, including convolutional neural networks (CNNs) for visual data and transformer networks for textual data, to extract and synthesize features from multiple sources. Key components of LMM architecture include modality encoders that transform raw data from multiple different sources into usable representations, input projectors that unify the multiple encoded data into shared embedding spaces, generative backbones that process multimodal inputs (e.g., LLM adapted to handle multimodal inputs), and output projectors that translate processed data into actionable formats. Multimodal generative models incorporate efficiency strategies such as shared embedding spaces to streamline processing, transfer learning to leverage pretraining across modalities, dynamic fusion techniques for task-specific optimization, and self-supervised learning to reduce reliance on annotated datasets. These innovations enhance their ability to integrate and process diverse data types effectively.

At step 815, a graphical user interface page is generated that provides the image analysis summarization result for the medical image(s) to an end user. The image analysis summarization result is based on the plurality of the results. In some implementations, the image analysis summarization result also provides a generic description of the image segment that includes insights, diagnosis or suggested conditions, conclusions, potential next steps, recommendations, and so on.

In some instances, process 800 may optionally further include step 820, step 825, and step 830. At step 820, an audio transcript data record associated with the patient is accessed. The audio transcript is generated from converting audio into text. The audio can be an audio recording of the interactions between healthcare providers during an MDT meeting and can be converted into a text transcript of the conversation. To convert audio into a text transcript, one or more machine-learning models such as an ASR model is utilized. In some implementations, the audio transcript data record is stored in a cloud service provider platform such as the platform 114 described above and/or in a database such as the databases 122 described above. In some implementations, the audio transcript data record is stored in the platform and/or the database in association with an electronic record or electronic records of a healthcare provider and/or in association with and/or within an electronic health record or electronic health records of the patient of the healthcare provider. In some implementations, the audio transcript data record is accessed from the platform and/or the database by a CDA, such as CDA 505 described with respect to FIG. 5A, where the CDA includes an application (e.g., CDA app with MDT-Assist) installed on, executing on, and/or accessed by one or more client devices.

At step 825, a dialogue segmentation result is obtained that identifies multiple participants in a dialogue recorded in the audio transcript data record from step 810. To obtain the dialogue segmentation result from the at least one machine learning model, the plurality of machine learning model prompts are used. A particular machine learning model prompt included in the one or more machine learning model prompts is generated, based at least on the audio transcript data record accessed in step 820. In addition to the dialogue segmentation result, the plurality of machine learning model prompts are also used to obtain one or more of an individual summarization result and an aggregated summarization result. The one or more of the individual summarization results comprise dialogue summarizations corresponding to each of the multiple participants identified in the dialogue segmentation result. The aggregated summarization result comprises an additional dialogue summarization corresponding to the dialogue among the multiple participants identified in the dialogue segmentation result. As a result, the one or more machine learning model prompts include at least one machine learning model prompt comprising the dialogue segmentation result, at least one machine learning model prompt comprising one or more of the individual summarization results, and at least one machine learning model prompt comprising the aggregated summarization result.

Once generated, the particular machine learning model prompt is provided to the at least one machine learning model. In some instances, the particular machine learning model prompt is (i) based at least on the audio transcript data record accessed in step 810, (ii) based at least on the dialogue segmentation result, (iii) based at least on the one or more individual summarization results, or (iv) based at least on the aggregated summarization result. In some instances, the dialogue segmentation result is received from the at least one machine learning model, where the dialogue segmentation result includes a) identification data for the multiple participants in the dialogue recorded in the audio transcript data record, b) the dialogue summarizations corresponding to each of the multiple participants identified in the dialogue segmentation result, and c) the additional dialogue summarization corresponding to the dialogue among the multiple participants identified in the dialogue segmentation result. In some instances, the identification data is based on personnel data describing one or more of the multiple participants. In some instances, (b) and (c) are determined based on a combination of the identification data and the audio transcript data record.

In some instances, a particular machine learning model prompt included in the one or more machine learning model prompts is generated, based at least on the dialogue segmentation result. Once generated, the particular machine learning model prompt is provided to the at least one machine learning model. The one or more of the individual summarization results and/or the aggregated summarization result are received from the at least one machine learning model. The individual summarization result comprises a respective individual summarization of respective dialogue of each participant of the multiple participants. The aggregated summarization result is based on a combination of the respective individual summarization for each participant.

At step 830, the graphical user interface page is modified to further provide the one or more individual summarization result and the aggregated summarization result.

### Illustrative System

As noted above, infrastructure as a service (IaaS) is one particular type of cloud computing. IaaS can be configured to provide virtualized computing resources over a public network (e.g., the Internet). In an IaaS model, a cloud computing provider can host the infrastructure components (e.g., servers, storage devices, network nodes (e.g., hardware), deployment software, platform virtualization (e.g., a hypervisor layer), or the like). In some cases, an IaaS provider may also supply a variety of services to accompany those infrastructure components (example services include billing software, monitoring software, logging software, load balancing software, clustering software, etc.). Thus, as these services may be policy-driven, IaaS users may be able to implement policies to drive load balancing to maintain application availability and performance.

In some instances, IaaS customers may access resources and services through a wide area network (WAN), such as the Internet, and can use the cloud provider's services to install the remaining elements of an application stack. For example, the user can log in to the IaaS platform to create virtual machines (VMs), install operating systems (OSs) on each VM, deploy middleware such as databases, create storage buckets for workloads and backups, and even install enterprise software into that VM. Customers can then use the provider's services to perform various functions, including balancing network traffic, troubleshooting application issues, monitoring performance, managing disaster recovery, etc.

In most cases, a cloud computing model will require the participation of a cloud provider. The cloud provider may, but need not be, a third-party service that specializes in providing (e.g., offering, renting, selling) IaaS. An entity might also opt to deploy a private cloud, becoming its own provider of infrastructure services.

In some examples, IaaS deployment is the process of putting a new application, or a new version of an application, onto a prepared application server or the like. It may also include the process of preparing the server (e.g., installing libraries, daemons, etc.). This is often managed by the cloud provider, below the hypervisor layer (e.g., the servers, storage, network hardware, and virtualization). Thus, the customer may be responsible for handling (OS), middleware, and/or application deployment (e.g., on self-service virtual machines (e.g., that can be spun up on demand)) or the like.

In some examples, IaaS provisioning may refer to acquiring computers or virtual hosts for use, and even installing needed libraries or services on them. In most cases, deployment does not include provisioning, and the provisioning may need to be performed first.

In some cases, there are two different challenges for IaaS provisioning. First, there is the initial challenge of provisioning the initial set of infrastructure before anything is running. Second, there is the challenge of evolving the existing infrastructure (e.g., adding new services, changing services, removing services, etc.) once everything has been provisioned. In some cases, these two challenges may be addressed by enabling the configuration of the infrastructure to be defined declaratively. In other words, the infrastructure (e.g., what components are needed and how they interact) can be defined by one or more configuration files. Thus, the overall topology of the infrastructure (e.g., what resources depend on which, and how they each work together) can be described declaratively. In some instances, once the topology is defined, a workflow can be generated that creates and/or manages the different components described in the configuration files.

In some examples, an infrastructure may have many interconnected elements. For example, there may be one or more virtual private clouds (VPCs) (e.g., a potentially on-demand pool of configurable and/or shared computing resources), also known as a core network. In some examples, there may also be one or more inbound/outbound traffic group rules provisioned to define how the inbound and/or outbound traffic of the network will be set up and one or more virtual machines (VMs). Other infrastructure elements may also be provisioned, such as a load balancer, a database, or the like. As more and more infrastructure elements are desired and/or added, the infrastructure may incrementally evolve.

In some instances, continuous deployment techniques may be employed to enable deployment of infrastructure code across various virtual computing environments. Additionally, the described techniques can enable infrastructure management within these environments. In some examples, service teams can write code that is desired to be deployed to one or more, but often many, different production environments (e.g., across various different geographic locations, sometimes spanning the entire world). However, in some examples, the infrastructure on which the code will be deployed must first be set up. In some instances, the provisioning can be done manually, a provisioning tool may be utilized to provision the resources, and/or deployment tools may be utilized to deploy the code once the infrastructure is provisioned.

FIG. 9 is a block diagram 900 illustrating an example pattern of an IaaS architecture, according to at least one embodiment. Service operators 902 can be communicatively coupled to a secure host tenancy 904 that can include a virtual cloud network (VCN) 906 and a secure host subnet 908. In some examples, the service operators 902 may be using one or more client computing devices, which may be portable handheld devices (e.g., an iPhone®, cellular telephone, an iPad®, computing tablet, a personal digital assistant (PDA)) or wearable devices (e.g., a Google Glass® head mounted display), running software such as Microsoft Windows Mobile®, and/or a variety of mobile operating systems such as iOS, Windows Phone, Android, BlackBerry 8, Palm OS, and the like, and being Internet, e-mail, short message service (SMS), Blackberry®, or other communication protocol enabled. Alternatively, the client computing devices can be general purpose personal computers including, by way of example, personal computers and/or laptop computers running various versions of Microsoft Windows®, Apple Macintosh®, and/or Linux operating systems. The client computing devices can be workstation computers running any of a variety of commercially-available UNIX® or UNIX-like operating systems, including without limitation the variety of GNU/Linux operating systems, such as for example, Google Chrome OS. Alternatively, or in addition, client computing devices may be any other electronic device, such as a thin-client computer, an Internet-enabled gaming system (e.g., a Microsoft Xbox gaming console with or without a Kinect® gesture input device), and/or a personal messaging device, capable of communicating over a network that can access the VCN 906 and/or the Internet.

The VCN 906 can include a local peering gateway (LPG) 910 that can be communicatively coupled to a secure shell (SSH) VCN 912 via an LPG 910 contained in the SSH VCN 912. The SSH VCN 912 can include an SSH subnet 914, and the SSH VCN 912 can be communicatively coupled to a control plane VCN 916 via the LPG 910 contained in the control plane VCN 916. Also, the SSH VCN 912 can be communicatively coupled to a data plane VCN 918 via an LPG 910. The control plane VCN 916 and the data plane VCN 918 can be contained in a service tenancy 919 that can be owned and/or operated by the IaaS provider.

The control plane VCN 916 can include a control plane demilitarized zone (DMZ) tier 920 that acts as a perimeter network (e.g., portions of a corporate network between the corporate intranet and external networks). The DMZ-based servers may have restricted responsibilities and help keep breaches contained. Additionally, the DMZ tier 920 can include one or more load balancer (LB) subnet(s) 922, a control plane app tier 924 that can include app subnet(s) 926, a control plane data tier 928 that can include database (DB) subnet(s) 930 (e.g., frontend DB subnet(s) and/or backend DB subnet(s)). The LB subnet(s) 922 contained in the control plane DMZ tier 920 can be communicatively coupled to the app subnet(s) 926 contained in the control plane app tier 924 and an Internet gateway 934 that can be contained in the control plane VCN 916, and the app subnet(s) 926 can be communicatively coupled to the DB subnet(s) 930 contained in the control plane data tier 928 and a service gateway 936 and a network address translation (NAT) gateway 938. The control plane VCN 916 can include the service gateway 936 and the NAT gateway 938.

The control plane VCN 916 can include a data plane mirror app tier 940 that can include app subnet(s) 926. The app subnet(s) 926 contained in the data plane mirror app tier 940 can include a virtual network interface controller (VNIC) 942 that can execute a compute instance 944. The compute instance 944 can communicatively couple the app subnet(s) 926 of the data plane mirror app tier 940 to app subnet(s) 926 that can be contained in a data plane app tier 946.

The data plane VCN 918 can include the data plane app tier 946, a data plane DMZ tier 948, and a data plane data tier 950. The data plane DMZ tier 948 can include LB subnet(s) 922 that can be communicatively coupled to the app subnet(s) 926 of the data plane app tier 946 and the Internet gateway 934 of the data plane VCN 918. The app subnet(s) 926 can be communicatively coupled to the service gateway 936 of the data plane VCN 918 and the NAT gateway 938 of the data plane VCN 918. The data plane data tier 950 can also include the DB subnet(s) 930 that can be communicatively coupled to the app subnet(s) 926 of the data plane app tier 946.

The Internet gateway 934 of the control plane VCN 916 and of the data plane VCN 918 can be communicatively coupled to a metadata management service 952 that can be communicatively coupled to public Internet 954. Public Internet 954 can be communicatively coupled to the NAT gateway 938 of the control plane VCN 916 and of the data plane VCN 918. The service gateway 936 of the control plane VCN 916 and of the data plane VCN 918 can be communicatively coupled to cloud services 956.

In some examples, the service gateway 936 of the control plane VCN 916 or of the data plane VCN 918 can make application programming interface (API) calls to cloud services 956 without going through public Internet 954. The API calls to cloud services 956 from the service gateway 936 can be one-way: the service gateway 936 can make API calls to cloud services 956, and cloud services 956 can send requested data to the service gateway 936. But, cloud services 956 may not initiate API calls to the service gateway 936.

In some examples, the secure host tenancy 904 can be directly connected to the service tenancy 919, which may be otherwise isolated. The secure host subnet 908 can communicate with the SSH subnet 914 through an LPG 910 that may enable two-way communication over an otherwise isolated system. Connecting the secure host subnet 908 to the SSH subnet 914 may give the secure host subnet 908 access to other entities within the service tenancy 919.

The control plane VCN 916 may allow users of the service tenancy 919 to set up or otherwise provision desired resources. Desired resources provisioned in the control plane VCN 916 may be deployed or otherwise used in the data plane VCN 918. In some examples, the control plane VCN 916 can be isolated from the data plane VCN 918, and the data plane mirror app tier 940 of the control plane VCN 916 can communicate with the data plane app tier 946 of the data plane VCN 918 via VNICs 942 that can be contained in the data plane mirror app tier 940 and the data plane app tier 946.

In some examples, users of the system, or customers, can make requests, for example create, read, update, or delete (CRUD) operations, through public Internet 954 that can communicate the requests to the metadata management service 952. The metadata management service 952 can communicate the request to the control plane VCN 916 through the Internet gateway 934. The request can be received by the LB subnet(s) 922 contained in the control plane DMZ tier 920. The LB subnet(s) 922 may determine that the request is valid, and in response to this determination, the LB subnet(s) 922 can transmit the request to app subnet(s) 926 contained in the control plane app tier 924. If the request is validated and requires a call to public Internet 954, the call to public Internet 954 may be transmitted to the NAT gateway 938 that can make the call to public Internet 954. Metadata that may be desired to be stored by the request can be stored in the DB subnet(s) 930.

In some examples, the data plane mirror app tier 940 can facilitate direct communication between the control plane VCN 916 and the data plane VCN 918. For example, changes, updates, or other suitable modifications to configuration may be desired to be applied to the resources contained in the data plane VCN 918. Via a VNIC 942, the control plane VCN 916 can directly communicate with, and can thereby execute the changes, updates, or other suitable modifications to configuration to, resources contained in the data plane VCN 918.

In some embodiments, the control plane VCN 916 and the data plane VCN 918 can be contained in the service tenancy 919. In this case, the user, or the customer, of the system may not own or operate either the control plane VCN 916 or the data plane VCN 918. Instead, the IaaS provider may own or operate the control plane VCN 916 and the data plane VCN 918, both of which may be contained in the service tenancy 919. This embodiment can enable isolation of networks that may prevent users or customers from interacting with other users', or other customers', resources. Also, this embodiment may allow users or customers of the system to store databases privately without needing to rely on public Internet 954, which may not have a desired level of threat prevention, for storage.

In other embodiments, the LB subnet(s) 922 contained in the control plane VCN 916 can be configured to receive a signal from the service gateway 936. In this embodiment, the control plane VCN 916 and the data plane VCN 918 may be configured to be called by a customer of the IaaS provider without calling public Internet 954. Customers of the IaaS provider may desire this embodiment since database(s) that the customers use may be controlled by the IaaS provider and may be stored on the service tenancy 919, which may be isolated from public Internet 954.

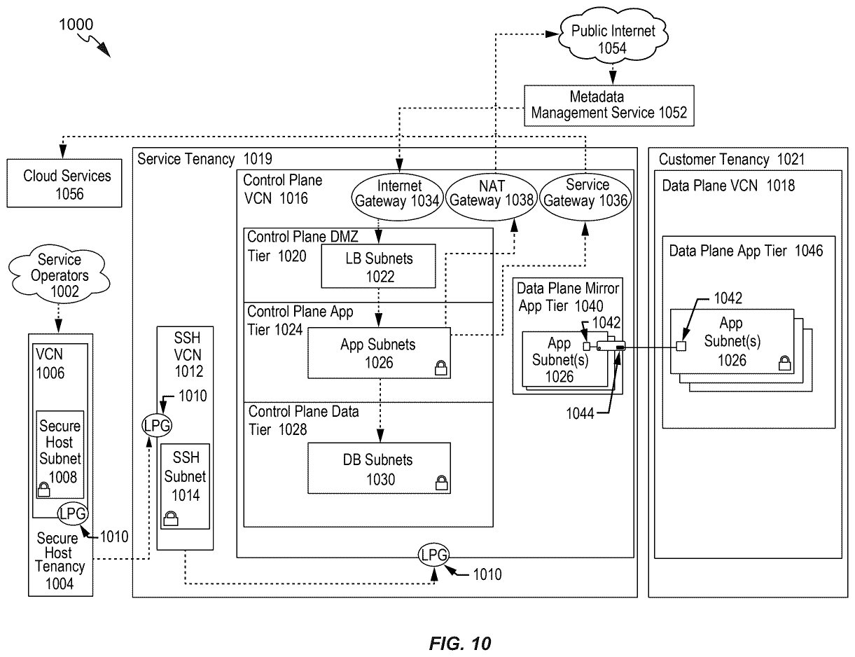

FIG. 10 is a block diagram 1000 illustrating another example pattern of an IaaS architecture, according to at least one embodiment. Service operators 1002 (e.g., service operators 902 of FIG. 9) can be communicatively coupled to a secure host tenancy 1004 (e.g., the secure host tenancy 904 of FIG. 9) that can include a virtual cloud network (VCN) 1006 (e.g., the VCN 906 of FIG. 9) and a secure host subnet 1008 (e.g., the secure host subnet 908 of FIG. 9). The VCN 1006 can include a local peering gateway (LPG) 1010 (e.g., the LPG 910 of FIG. 9) that can be communicatively coupled to a secure shell (SSH) VCN 1012 (e.g., the SSH VCN 912 of FIG. 9) via an LPG 910 contained in the SSH VCN 1012. The SSH VCN 1012 can include an SSH subnet 1014 (e.g., the SSH subnet 914 of FIG. 9), and the SSH VCN 1012 can be communicatively coupled to a control plane VCN 1016 (e.g., the control plane VCN 916 of FIG. 9) via an LPG 1010 contained in the control plane VCN 1016. The control plane VCN 1016 can be contained in a service tenancy 1019 (e.g., the service tenancy 919 of FIG. 9), and the data plane VCN 1018 (e.g., the data plane VCN 918 of FIG. 9) can be contained in a customer tenancy 1021 that may be owned or operated by users, or customers, of the system.

The control plane VCN 1016 can include a control plane DMZ tier 1020 (e.g., the control plane DMZ tier 920 of FIG. 9) that can include LB subnet(s) 1022 (e.g., LB subnet(s) 922 of FIG. 9), a control plane app tier 1024 (e.g., the control plane app tier 924 of FIG. 9) that can include app subnet(s) 1026 (e.g., app subnet(s) 926 of FIG. 9), a control plane data tier 1028 (e.g., the control plane data tier 928 of FIG. 9) that can include database (DB) subnet(s) 1030 (e.g., similar to DB subnet(s) 930 of FIG. 9). The LB subnet(s) 1022 contained in the control plane DMZ tier 1020 can be communicatively coupled to the app subnet(s) 1026 contained in the control plane app tier 1024 and an Internet gateway 1034 (e.g., the Internet gateway 934 of FIG. 9) that can be contained in the control plane VCN 1016, and the app subnet(s) 1026 can be communicatively coupled to the DB subnet(s) 1030 contained in the control plane data tier 1028 and a service gateway 1036 (e.g., the service gateway 936 of FIG. 9) and a network address translation (NAT) gateway 1038 (e.g., the NAT gateway 938 of FIG. 9). The control plane VCN 1016 can include the service gateway 1036 and the NAT gateway 1038.

The control plane VCN 1016 can include a data plane mirror app tier 1040 (e.g., the data plane mirror app tier 940 of FIG. 9) that can include app subnet(s) 1026. The app subnet(s) 1026 contained in the data plane mirror app tier 1040 can include a virtual network interface controller (VNIC) 1042 (e.g., the VNIC of 942) that can execute a compute instance 1044 (e.g., similar to the compute instance 944 of FIG. 9). The compute instance 1044 can facilitate communication between the app subnet(s) 1026 of the data plane mirror app tier 1040 and the app subnet(s) 1026 that can be contained in a data plane app tier 1046 (e.g., the data plane app tier 946 of FIG. 9) via the VNIC 1042 contained in the data plane mirror app tier 1040 and the VNIC 1042 contained in the data plane app tier 1046.

The Internet gateway 1034 contained in the control plane VCN 1016 can be communicatively coupled to a metadata management service 1052 (e.g., the metadata management service 952 of FIG. 9) that can be communicatively coupled to public Internet 1054 (e.g., public Internet 954 of FIG. 9). Public Internet 1054 can be communicatively coupled to the NAT gateway 1038 contained in the control plane VCN 1016. The service gateway 1036 contained in the control plane VCN 1016 can be communicatively coupled to cloud services 1056 (e.g., cloud services 956 of FIG. 9).

In some examples, the data plane VCN 1018 can be contained in the customer tenancy 1021. In this case, the IaaS provider may provide the control plane VCN 1016 for each customer, and the IaaS provider may, for each customer, set up a unique compute instance 1044 that is contained in the service tenancy 1019. Each compute instance 1044 may allow communication between the control plane VCN 1016, contained in the service tenancy 1019, and the data plane VCN 1018 that is contained in the customer tenancy 1021. The compute instance 1044 may allow resources, that are provisioned in the control plane VCN 1016 that is contained in the service tenancy 1019, to be deployed or otherwise used in the data plane VCN 1018 that is contained in the customer tenancy 1021.

In other examples, the customer of the IaaS provider may have databases that live in the customer tenancy 1021. In this example, the control plane VCN 1016 can include the data plane mirror app tier 1040 that can include app subnet(s) 1026. The data plane mirror app tier 1040 can reside in the data plane VCN 1018, but the data plane mirror app tier 1040 may not live in the data plane VCN 1018. That is, the data plane mirror app tier 1040 may have access to the customer tenancy 1021, but the data plane mirror app tier 1040 may not exist in the data plane VCN 1018 or be owned or operated by the customer of the IaaS provider. The data plane mirror app tier 1040 may be configured to make calls to the data plane VCN 1018 but may not be configured to make calls to any entity contained in the control plane VCN 1016. The customer may desire to deploy or otherwise use resources in the data plane VCN 1018 that are provisioned in the control plane VCN 1016, and the data plane mirror app tier 1040 can facilitate the desired deployment, or other usage of resources, of the customer.

In some embodiments, the customer of the IaaS provider can apply filters to the data plane VCN 1018. In this embodiment, the customer can determine what the data plane VCN 1018 can access, and the customer may restrict access to public Internet 1054 from the data plane VCN 1018. The IaaS provider may not be able to apply filters or otherwise control access of the data plane VCN 1018 to any outside networks or databases. Applying filters and controls by the customer onto the data plane VCN 1018, contained in the customer tenancy 1021, can help isolate the data plane VCN 1018 from other customers and from public Internet 1054.

In some embodiments, cloud services 1056 can be called by the service gateway 1036 to access services that may not exist on public Internet 1054, on the control plane VCN 1016, or on the data plane VCN 1018. The connection between cloud services 1056 and the control plane VCN 1016 or the data plane VCN 1018 may not be live or continuous. Cloud services 1056 may exist on a different network owned or operated by the IaaS provider. Cloud services 1056 may be configured to receive calls from the service gateway 1036 and may be configured to not receive calls from public Internet 1054. Some cloud services 1056 may be isolated from other cloud services 1056, and the control plane VCN 1016 may be isolated from cloud services 1056 that may not be in the same region as the control plane VCN 1016. For example, the control plane VCN 1016 may be located in “Region 1,” and cloud service “Deployment 9,” may be located in Region 1 and in “Region 2.” If a call to Deployment 9 is made by the service gateway 1036 contained in the control plane VCN 1016 located in Region 1, the call may be transmitted to Deployment 9 in Region 1. In this example, the control plane VCN 1016, or Deployment 9 in Region 1, may not be communicatively coupled to, or otherwise in communication with, Deployment 9 in Region 2.

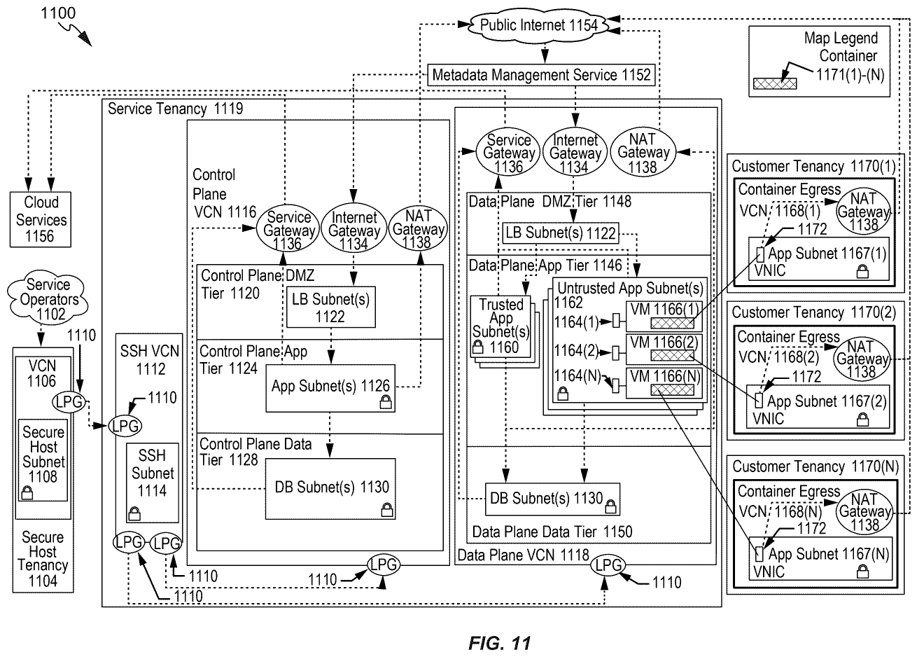

FIG. 11 is a block diagram 1100 illustrating another example pattern of an IaaS architecture, according to at least one embodiment. Service operators 1102 (e.g., service operators 902 of FIG. 9) can be communicatively coupled to a secure host tenancy 1104 (e.g., the secure host tenancy 904 of FIG. 9) that can include a virtual cloud network (VCN) 1106 (e.g., the VCN 906 of FIG. 9) and a secure host subnet 1108 (e.g., the secure host subnet 908 of FIG. 9). The VCN 1106 can include an LPG 1110 (e.g., the LPG 910 of FIG. 9) that can be communicatively coupled to an SSH VCN 1112 (e.g., the SSH VCN 912 of FIG. 9) via an LPG 1110 contained in the SSH VCN 1112. The SSH VCN 1112 can include an SSH subnet 1114 (e.g., the SSH subnet 914 of FIG. 9), and the SSH VCN 1112 can be communicatively coupled to a control plane VCN 1116 (e.g., the control plane VCN 916 of FIG. 9) via an LPG 1110 contained in the control plane VCN 1116 and to a data plane VCN 1118 (e.g., the data plane 918 of FIG. 9) via an LPG 1110 contained in the data plane VCN 1118. The control plane VCN 1116 and the data plane VCN 1118 can be contained in a service tenancy 1119 (e.g., the service tenancy 919 of FIG. 9).

The control plane VCN 1116 can include a control plane DMZ tier 1120 (e.g., the control plane DMZ tier 920 of FIG. 9) that can include load balancer (LB) subnet(s) 1122 (e.g., LB subnet(s) 922 of FIG. 9), a control plane app tier 1124 (e.g., the control plane app tier 924 of FIG. 9) that can include app subnet(s) 1126 (e.g., similar to app subnet(s) 926 of FIG. 9), a control plane data tier 1128 (e.g., the control plane data tier 928 of FIG. 9) that can include DB subnet(s) 1130. The LB subnet(s) 1122 contained in the control plane DMZ tier 1120 can be communicatively coupled to the app subnet(s) 1126 contained in the control plane app tier 1124 and to an Internet gateway 1134 (e.g., the Internet gateway 934 of FIG. 9) that can be contained in the control plane VCN 1116, and the app subnet(s) 1126 can be communicatively coupled to the DB subnet(s) 1130 contained in the control plane data tier 1128 and to a service gateway 1136 (e.g., the service gateway of FIG. 9) and a network address translation (NAT) gateway 1138 (e.g., the NAT gateway 938 of FIG. 9). The control plane VCN 1116 can include the service gateway 1136 and the NAT gateway 1138.

The data plane VCN 1118 can include a data plane app tier 1146 (e.g., the data plane app tier 946 of FIG. 9), a data plane DMZ tier 1148 (e.g., the data plane DMZ tier 948 of FIG. 9), and a data plane data tier 1150 (e.g., the data plane data tier 950 of FIG. 9). The data plane DMZ tier 1148 can include LB subnet(s) 1122 that can be communicatively coupled to trusted app subnet(s) 1160 and untrusted app subnet(s) 1162 of the data plane app tier 1146 and the Internet gateway 1134 contained in the data plane VCN 1118. The trusted app subnet(s) 1160 can be communicatively coupled to the service gateway 1136 contained in the data plane VCN 1118, the NAT gateway 1138 contained in the data plane VCN 1118, and DB subnet(s) 1130 contained in the data plane data tier 1150. The untrusted app subnet(s) 1162 can be communicatively coupled to the service gateway 1136 contained in the data plane VCN 1118 and DB subnet(s) 1130 contained in the data plane data tier 1150. The data plane data tier 1150 can include DB subnet(s) 1130 that can be communicatively coupled to the service gateway 1136 contained in the data plane VCN 1118.

The untrusted app subnet(s) 1162 can include one or more primary VNICs 1164(1)-(N) that can be communicatively coupled to tenant virtual machines (VMs) 1166(1)-(N). Each tenant VM 1166(1)-(N) can be communicatively coupled to a respective app subnet 1167(1)-(N) that can be contained in respective container egress VCNs 1168(1)-(N) that can be contained in respective customer tenancies 1170(1)-(N). Respective secondary VNICs 1172(1)-(N) can facilitate communication between the untrusted app subnet(s) 1162 contained in the data plane VCN 1118 and the app subnet contained in the container egress VCNs 1168(1)-(N). Each container egress VCNs 1168(1)-(N) can include a NAT gateway 1138 that can be communicatively coupled to public Internet 1154 (e.g., public Internet 954 of FIG. 9).

The Internet gateway 1134 contained in the control plane VCN 1116 and contained in the data plane VCN 1118 can be communicatively coupled to a metadata management service 1152 (e.g., the metadata management system 952 of FIG. 9) that can be communicatively coupled to public Internet 1154. Public Internet 1154 can be communicatively coupled to the NAT gateway 1138 contained in the control plane VCN 1116 and contained in the data plane VCN 1118. The service gateway 1136 contained in the control plane VCN 1116 and contained in the data plane VCN 1118 can be communicatively coupled to cloud services 1156.

In some embodiments, the data plane VCN 1118 can be integrated with customer tenancies 1170. This integration can be useful or desirable for customers of the IaaS provider in some cases such as a case that may desire support when executing code. The customer may provide code to run that may be destructive, may communicate with other customer resources, or may otherwise cause undesirable effects. In response to this, the IaaS provider may determine whether to run code given to the IaaS provider by the customer.

In some examples, the customer of the IaaS provider may grant temporary network access to the IaaS provider and request a function to be attached to the data plane app tier 1146. Code to run the function may be executed in the VMs 1166(1)-(N), and the code may not be configured to run anywhere else on the data plane VCN 1118. Each VM 1166(1)-(N) may be connected to one customer tenancy 1170. Respective containers 1171(1)-(N) contained in the VMs 1166(1)-(N) may be configured to run the code. In this case, there can be a dual isolation (e.g., the containers 1171(1)-(N) running code, where the containers 1171(1)-(N) may be contained in at least the VM 1166(1)-(N) that are contained in the untrusted app subnet(s) 1162), which may help prevent incorrect or otherwise undesirable code from damaging the network of the IaaS provider or from damaging a network of a different customer. The containers 1171(1)-(N) may be communicatively coupled to the customer tenancy 1170 and may be configured to transmit or receive data from the customer tenancy 1170. The containers 1171(1)-(N) may not be configured to transmit or receive data from any other entity in the data plane VCN 1118. Upon completion of running the code, the IaaS provider may kill or otherwise dispose of the containers 1171(1)-(N).

In some embodiments, the trusted app subnet(s) 1160 may run code that may be owned or operated by the IaaS provider. In this embodiment, the trusted app subnet(s) 1160 may be communicatively coupled to the DB subnet(s) 1130 and be configured to execute CRUD operations in the DB subnet(s) 1130. The untrusted app subnet(s) 1162 may be communicatively coupled to the DB subnet(s) 1130, but in this embodiment, the untrusted app subnet(s) may be configured to execute read operations in the DB subnet(s) 1130. The containers 1171(1)-(N) that can be contained in the VM 1166(1)-(N) of each customer and that may run code from the customer may not be communicatively coupled with the DB subnet(s) 1130.

In other embodiments, the control plane VCN 1116 and the data plane VCN 1118 may not be directly communicatively coupled. In this embodiment, there may be no direct communication between the control plane VCN 1116 and the data plane VCN 1118. However, communication can occur indirectly through at least one method. An LPG 1110 may be established by the IaaS provider that can facilitate communication between the control plane VCN 1116 and the data plane VCN 1118. In another example, the control plane VCN 1116 or the data plane VCN 1118 can make a call to cloud services 1156 via the service gateway 1136. For example, a call to cloud services 1156 from the control plane VCN 1116 can include a request for a service that can communicate with the data plane VCN 1118.

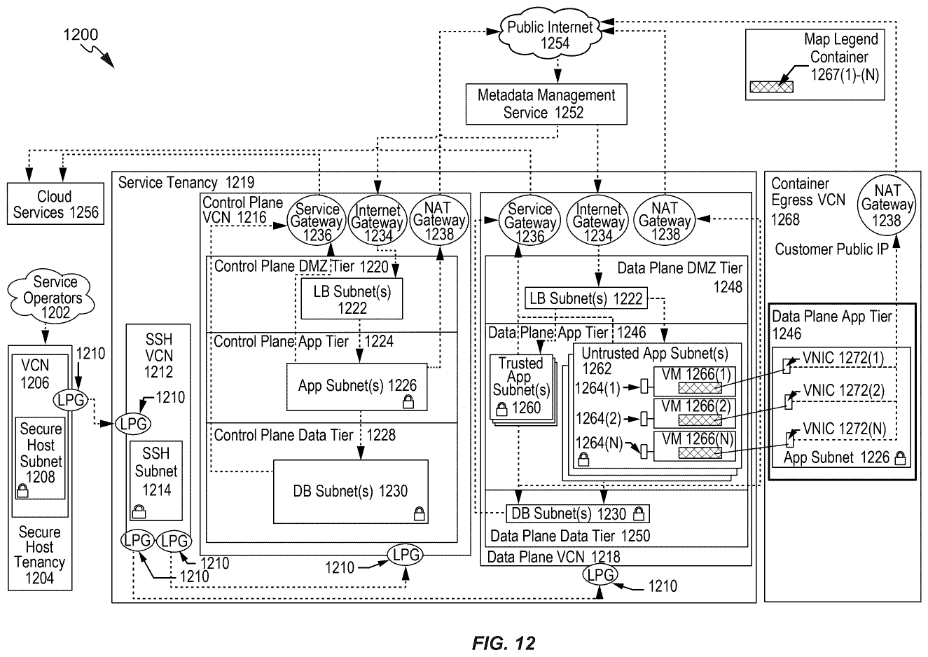

FIG. 12 is a block diagram 1200 illustrating another example pattern of an IaaS architecture, according to at least one embodiment. Service operators 1202 (e.g., service operators 902 of FIG. 9) can be communicatively coupled to a secure host tenancy 1204 (e.g., the secure host tenancy 904 of FIG. 9) that can include a virtual cloud network (VCN) 1206 (e.g., the VCN 906 of FIG. 9) and a secure host subnet 1208 (e.g., the secure host subnet 908 of FIG. 9). The VCN 1206 can include an LPG 1210 (e.g., the LPG 910 of FIG. 9) that can be communicatively coupled to an SSH VCN 1212 (e.g., the SSH VCN 912 of FIG. 9) via an LPG 1210 contained in the SSH VCN 1212. The SSH VCN 1212 can include an SSH subnet 1214 (e.g., the SSH subnet 914 of FIG. 9), and the SSH VCN 1212 can be communicatively coupled to a control plane VCN 1216 (e.g., the control plane VCN 916 of FIG. 9) via an LPG 1210 contained in the control plane VCN 1216 and to a data plane VCN 1218 (e.g., the data plane 918 of FIG. 9) via an LPG 1210 contained in the data plane VCN 1218. The control plane VCN 1216 and the data plane VCN 1218 can be contained in a service tenancy 1219 (e.g., the service tenancy 919 of FIG. 9).

The control plane VCN 1216 can include a control plane DMZ tier 1220 (e.g., the control plane DMZ tier 920 of FIG. 9) that can include LB subnet(s) 1222 (e.g., LB subnet(s) 922 of FIG. 9), a control plane app tier 1224 (e.g., the control plane app tier 924 of FIG. 9) that can include app subnet(s) 1226 (e.g., app subnet(s) 926 of FIG. 9), a control plane data tier 1228 (e.g., the control plane data tier 928 of FIG. 9) that can include DB subnet(s) 1230 (e.g., DB subnet(s) 1130 of FIG. 11). The LB subnet(s) 1222 contained in the control plane DMZ tier 1220 can be communicatively coupled to the app subnet(s) 1226 contained in the control plane app tier 1224 and to an Internet gateway 1234 (e.g., the Internet gateway 934 of FIG. 9) that can be contained in the control plane VCN 1216, and the app subnet(s) 1226 can be communicatively coupled to the DB subnet(s) 1230 contained in the control plane data tier 1228 and to a service gateway 1236 (e.g., the service gateway of FIG. 9) and a network address translation (NAT) gateway 1238 (e.g., the NAT gateway 938 of FIG. 9). The control plane VCN 1216 can include the service gateway 1236 and the NAT gateway 1238.

The data plane VCN 1218 can include a data plane app tier 1246 (e.g., the data plane app tier 946 of FIG. 9), a data plane DMZ tier 1248 (e.g., the data plane DMZ tier 948 of FIG. 9), and a data plane data tier 1250 (e.g., the data plane data tier 950 of FIG. 9). The data plane DMZ tier 1248 can include LB subnet(s) 1222 that can be communicatively coupled to trusted app subnet(s) 1260 (e.g., trusted app subnet(s) 1160 of FIG. 11) and untrusted app subnet(s) 1262 (e.g., untrusted app subnet(s) 1162 of FIG. 11) of the data plane app tier 1246 and the Internet gateway 1234 contained in the data plane VCN 1218. The trusted app subnet(s) 1260 can be communicatively coupled to the service gateway 1236 contained in the data plane VCN 1218, the NAT gateway 1238 contained in the data plane VCN 1218, and DB subnet(s) 1230 contained in the data plane data tier 1250. The untrusted app subnet(s) 1262 can be communicatively coupled to the service gateway 1236 contained in the data plane VCN 1218 and DB subnet(s) 1230 contained in the data plane data tier 1250. The data plane data tier 1250 can include DB subnet(s) 1230 that can be communicatively coupled to the service gateway 1236 contained in the data plane VCN 1218.

The untrusted app subnet(s) 1262 can include primary VNICs 1264(1)-(N) that can be communicatively coupled to tenant virtual machines (VMs) 1266(1)-(N) residing within the untrusted app subnet(s) 1262. Each tenant VM 1266(1)-(N) can run code in a respective container 1267(1)-(N), and be communicatively coupled to an app subnet 1226 that can be contained in a data plane app tier 1246 that can be contained in a container egress VCN 1268. Respective secondary VNICs 1272(1)-(N) can facilitate communication between the untrusted app subnet(s) 1262 contained in the data plane VCN 1218 and the app subnet contained in the container egress VCN 1268. The container egress VCN can include a NAT gateway 1238 that can be communicatively coupled to public Internet 1254 (e.g., public Internet 954 of FIG. 9).

The Internet gateway 1234 contained in the control plane VCN 1216 and contained in the data plane VCN 1218 can be communicatively coupled to a metadata management service 1252 (e.g., the metadata management system 952 of FIG. 9) that can be communicatively coupled to public Internet 1254. Public Internet 1254 can be communicatively coupled to the NAT gateway 1238 contained in the control plane VCN 1216 and contained in the data plane VCN 1218. The service gateway 1236 contained in the control plane VCN 1216 and contained in the data plane VCN 1218 can be communicatively coupled to cloud services 1256.

In some examples, the pattern illustrated by the architecture of block diagram 1200 of FIG. 12 may be considered an exception to the pattern illustrated by the architecture of block diagram 1100 of FIG. 11 and may be desirable for a customer of the IaaS provider if the IaaS provider cannot directly communicate with the customer (e.g., a disconnected region). The respective containers 1267(1)-(N) that are contained in the VMs 1266(1)-(N) for each customer can be accessed in real-time by the customer. The containers 1267(1)-(N) may be configured to make calls to respective secondary VNICs 1272(1)-(N) contained in app subnet(s) 1226 of the data plane app tier 1246 that can be contained in the container egress VCN 1268. The secondary VNICs 1272(1)-(N) can transmit the calls to the NAT gateway 1238 that may transmit the calls to public Internet 1254. In this example, the containers 1267(1)-(N) that can be accessed in real-time by the customer can be isolated from the control plane VCN 1216 and can be isolated from other entities contained in the data plane VCN 1218. The containers 1267(1)-(N) may also be isolated from resources from other customers.

In other examples, the customer can use the containers 1267(1)-(N) to call cloud services 1256. In this example, the customer may run code in the containers 1267(1)-(N) that requests a service from cloud services 1256. The containers 1267(1)-(N) can transmit this request to the secondary VNICs 1272(1)-(N) that can transmit the request to the NAT gateway that can transmit the request to public Internet 1254. Public Internet 1254 can transmit the request to LB subnet(s) 1222 contained in the control plane VCN 1216 via the Internet gateway 1234. In response to determining the request is valid, the LB subnet(s) can transmit the request to app subnet(s) 1226 that can transmit the request to cloud services 1256 via the service gateway 1236.

It should be appreciated that IaaS architectures 900, 1000, 1100, 1200 depicted in the figures may have other components than those depicted. Further, the embodiments shown in the figures are only some examples of a cloud infrastructure system that may incorporate an embodiment of the disclosure. In some other embodiments, the IaaS systems may have more or fewer components than shown in the figures, may combine two or more components, or may have a different configuration or arrangement of components.

In certain embodiments, the IaaS systems described herein may include a suite of applications, middleware, and database service offerings that are delivered to a customer in a self-service, subscription-based, elastically scalable, reliable, highly available, and secure manner. An example of such an IaaS system is the Oracle Cloud Infrastructure (OCI) provided by the present assignee.

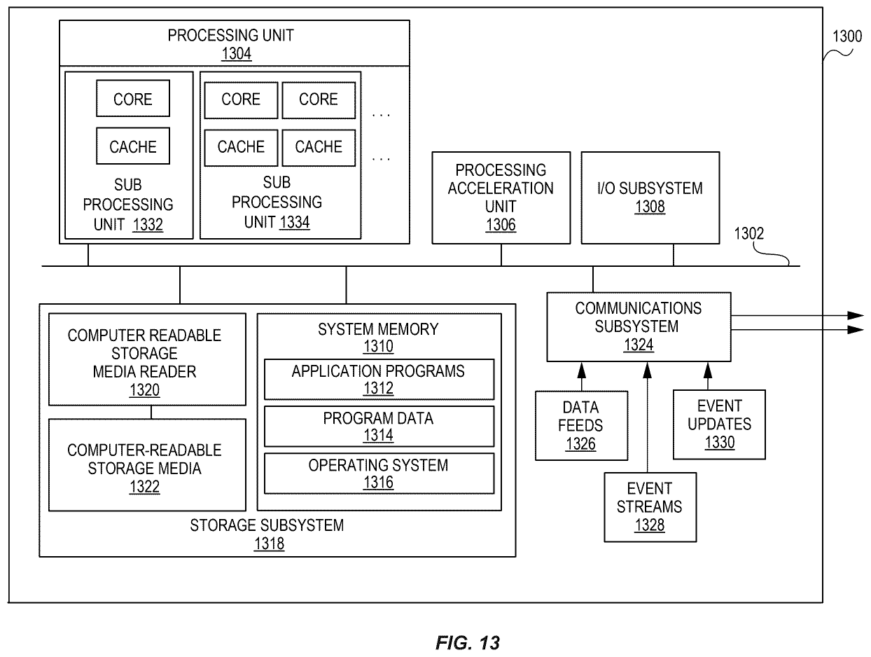

FIG. 13 illustrates an example computer system 1300, in which various embodiments may be implemented. The system 1300 may be used to implement any of the computer systems described above. As shown in the figure, computer system 1300 includes a processing unit 1304 that communicates with a number of peripheral subsystems via a bus subsystem 1302. These peripheral subsystems may include a processing acceleration unit 1306, an I/O subsystem 1308, a storage subsystem 1318 and a communications subsystem 1324. Storage subsystem 1318 includes tangible computer-readable storage media 1322 and a system memory 1310.

Bus subsystem 1302 provides a mechanism for letting the various components and subsystems of computer system 1300 communicate with each other as intended. Although bus subsystem 1302 is shown schematically as a single bus, alternative embodiments of the bus subsystem may utilize multiple buses. Bus subsystem 1302 may be any of several types of bus structures including a memory bus or memory controller, a peripheral bus, and a local bus using any of a variety of bus architectures. For example, such architectures may include an Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus, which can be implemented as a Mezzanine bus manufactured to the IEEE P1386.1 standard.

Processing unit 1304, which can be implemented as one or more integrated circuits (e.g., a conventional microprocessor or microcontroller), controls the operation of computer system 1300. One or more processors may be included in processing unit 1304. These processors may include single core or multicore processors. In certain embodiments, processing unit 1304 may be implemented as one or more independent processing units 1332 and/or 1334 with single or multicore processors included in each processing unit. In other embodiments, processing unit 1304 may also be implemented as a quad-core processing unit formed by integrating two dual-core processors into a single chip.

In various embodiments, processing unit 1304 can execute a variety of programs in response to program code and can maintain multiple concurrently executing programs or processes. At any given time, some or all of the program code to be executed can be resident in processor(s) 1304 and/or in storage subsystem 1318. Through suitable programming, processor(s) 1304 can provide various functionalities described above. Computer system 1300 may additionally include a processing acceleration unit 1306, which can include a digital signal processor (DSP), a special-purpose processor, and/or the like.

I/O subsystem 1308 may include user interface input devices and user interface output devices. User interface input devices may include a keyboard, pointing devices such as a mouse or trackball, a touchpad or touch screen incorporated into a display, a scroll wheel, a click wheel, a dial, a button, a switch, a keypad, audio input devices with voice command recognition systems, microphones, and other types of input devices. User interface input devices may include, for example, motion sensing and/or gesture recognition devices such as the Microsoft Kinect® motion sensor that enables users to control and interact with an input device, such as the Microsoft Xbox® 360 game controller, through a natural user interface using gestures and spoken commands. User interface input devices may also include eye gesture recognition devices such as the Google Glass® blink detector that detects eye activity (e.g., ‘blinking’ while taking pictures and/or making a menu selection) from users and transforms the eye gestures as input into an input device (e.g., Google Glass®). Additionally, user interface input devices may include voice recognition sensing devices that enable users to interact with voice recognition systems (e.g., Siri® navigator), through voice commands.

User interface input devices may also include, without limitation, three dimensional (3D) mice, joysticks or pointing sticks, gamepads and graphic tablets, and audio/visual devices such as speakers, digital cameras, digital camcorders, portable media players, webcams, image scanners, fingerprint scanners, barcode reader 3D scanners, 3D printers, laser rangefinders, and eye gaze tracking devices. Additionally, user interface input devices may include, for example, medical imaging input devices such as computed tomography, magnetic resonance imaging, position emission tomography, medical ultrasonography devices. User interface input devices may also include, for example, audio input devices such as MIDI keyboards, digital musical instruments and the like.

User interface output devices may include a display subsystem, indicator lights, or non-visual displays such as audio output devices, etc. The display subsystem may be a cathode ray tube (CRT), a flat-panel device, such as that using a liquid crystal display (LCD) or plasma display, a projection device, a touch screen, and the like. In general, use of the term “output device” is intended to include all possible types of devices and mechanisms for outputting information from computer system 1300 to a user or other computer. For example, user interface output devices may include, without limitation, a variety of display devices that visually convey text, graphics and audio/video information such as monitors, printers, speakers, headphones, automotive navigation systems, plotters, voice output devices, and modems.

Computer system 1300 may comprise a storage subsystem 1318 that provides a tangible non-transitory computer-readable storage medium for storing software and data constructs that provide the functionality of the embodiments described in this disclosure. The software can include programs, code modules, instructions, scripts, etc., that when executed by one or more cores or processors of processing unit 1304 provide the functionality described above. Storage subsystem 1318 may also provide a repository for storing data used in accordance with the present disclosure.

As depicted in the example in FIG. 13, storage subsystem 1318 can include various components including a system memory 1310, computer-readable storage media 1322, and a computer readable storage media reader 1320. System memory 1310 may store program instructions that are loadable and executable by processing unit 1304. System memory 1310 may also store data that is used during the execution of the instructions and/or data that is generated during the execution of the program instructions. Various different kinds of programs may be loaded into system memory 1310 including but not limited to client applications, Web browsers, mid-tier applications, relational database management systems (RDBMS), virtual machines, containers, etc.

System memory 1310 may also store an operating system 1316. Examples of operating system 1316 may include various versions of Microsoft Windows®, Apple Macintosh®, and/or Linux operating systems, a variety of commercially-available UNIX® or UNIX-like operating systems (including without limitation the variety of GNU/Linux operating systems, the Google Chrome® OS, and the like) and/or mobile operating systems such as iOS, Windows® Phone, Android® OS, BlackBerry® OS, and Palm® OS operating systems. In certain implementations where computer system 1300 executes one or more virtual machines, the virtual machines along with their guest operating systems (GOSs) may be loaded into system memory 1310 and executed by one or more processors or cores of processing unit 1304.

System memory 1310 can come in different configurations depending upon the type of computer system 1300. For example, system memory 1310 may be volatile memory (such as random access memory (RAM)) and/or non-volatile memory (such as read-only memory (ROM), flash memory, etc.) Different types of RAM configurations may be provided including a static random access memory (SRAM), a dynamic random access memory (DRAM), and others. In some implementations, system memory 1310 may include a basic input/output system (BIOS) containing basic routines that help to transfer information between elements within computer system 1300, such as during start-up.

Computer-readable storage media 1322 may represent remote, local, fixed, and/or removable storage devices plus storage media for temporarily and/or more permanently containing, storing, computer-readable information for use by computer system 1300 including instructions executable by processing unit 1304 of computer system 1300.

Computer-readable storage media 1322 can include any appropriate media known or used in the art, including storage media and communication media, such as but not limited to, volatile and non-volatile, removable and non-removable media implemented in any method or technology for storage and/or transmission of information. This can include tangible computer-readable storage media such as RAM, ROM, electronically erasable programmable ROM (EEPROM), flash memory or other memory technology, CD-ROM, digital versatile disk (DVD), or other optical storage, magnetic cassettes, magnetic tape, magnetic disk storage or other magnetic storage devices, or other tangible computer readable media.

By way of example, computer-readable storage media 1322 may include a hard disk drive that reads from or writes to non-removable, nonvolatile magnetic media, a magnetic disk drive that reads from or writes to a removable, nonvolatile magnetic disk, and an optical disk drive that reads from or writes to a removable, nonvolatile optical disk such as a CD ROM, DVD, and Blu-Ray® disk, or other optical media. Computer-readable storage media 1322 may include, but is not limited to, Zip® drives, flash memory cards, universal serial bus (USB) flash drives, secure digital (SD) cards, DVD disks, digital video tape, and the like. Computer-readable storage media 1322 may also include, solid-state drives (SSD) based on non-volatile memory such as flash-memory based SSDs, enterprise flash drives, solid state ROM, and the like, SSDs based on volatile memory such as solid state RAM, dynamic RAM, static RAM, DRAM-based SSDs, magnetoresistive RAM (MRAM) SSDs, and hybrid SSDs that use a combination of DRAM and flash memory based SSDs. The disk drives and their associated computer-readable media may provide non-volatile storage of computer-readable instructions, data structures, program modules, and other data for computer system 1300.

Machine-readable instructions executable by one or more processors or cores of processing unit 1304 may be stored on a non-transitory computer-readable storage medium. A non-transitory computer-readable storage medium can include physically tangible memory or storage devices that include volatile memory storage devices and/or non-volatile storage devices. Examples of non-transitory computer-readable storage medium include magnetic storage media (e.g., disk or tapes), optical storage media (e.g., DVDs, CDs), various types of RAM, ROM, or flash memory, hard drives, floppy drives, detachable memory drives (e.g., USB drives), or other type of storage device.

Communications subsystem 1324 provides an interface to other computer systems and networks. Communications subsystem 1324 serves as an interface for receiving data from and transmitting data to other systems from computer system 1300. For example, communications subsystem 1324 may enable computer system 1300 to connect to one or more devices via the Internet. In some embodiments communications subsystem 1324 can include radio frequency (RF) transceiver components for accessing wireless voice and/or data networks (e.g., using cellular telephone technology, advanced data network technology, such as 3G, 4G or EDGE (enhanced data rates for global evolution), WiFi (IEEE 802.11 family standards, or other mobile communication technologies, or any combination thereof)), global positioning system (GPS) receiver components, and/or other components. In some embodiments communications subsystem 1324 can provide wired network connectivity (e.g., Ethernet) in addition to or instead of a wireless interface.

In some embodiments, communications subsystem 1324 may also receive input communication in the form of structured and/or unstructured data feeds 1326, event streams 1328, event updates 1330, and the like on behalf of one or more users who may use computer system 1300.

By way of example, communications subsystem 1324 may be configured to receive data feeds 1326 in real-time from users of social networks and/or other communication services such as Twitter® feeds, Facebook® updates, web feeds such as Rich Site Summary (RSS) feeds, and/or real-time updates from one or more third party information sources.

Additionally, communications subsystem 1324 may also be configured to receive data in the form of continuous data streams, which may include event streams 1328 of real-time events and/or event updates 1330, that may be continuous or unbounded in nature with no explicit end. Examples of applications that generate continuous data may include, for example, sensor data applications, financial tickers, network performance measuring tools (e.g., network monitoring and traffic management applications), clickstream analysis tools, automobile traffic monitoring, and the like.

Communications subsystem 1324 may also be configured to output the structured and/or unstructured data feeds 1326, event streams 1328, event updates 1330, and the like to one or more databases that may be in communication with one or more streaming data source computers coupled to computer system 1300.

Computer system 1300 can be one of various types, including a handheld portable device (e.g., an iPhone® cellular phone, an iPad® computing tablet, a PDA), a wearable device (e.g., a Google Glass® head mounted display), a PC, a workstation, a mainframe, a kiosk, a server rack, or any other data processing system.

Due to the ever-changing nature of computers and networks, the description of computer system 1300 depicted in the figure is intended only as a specific example. Many other configurations having more or fewer components than the system depicted in the figure are possible. For example, customized hardware might also be used and/or particular elements might be implemented in hardware, firmware, software (including applets), or a combination. Further, connection to other computing devices, such as network input/output devices, may be employed. Based on the disclosure and teachings provided herein, a person of ordinary skill in the art will appreciate other ways and/or methods to implement the various embodiments.

Although specific embodiments have been described, various modifications, alterations, alternative constructions, and equivalents are also encompassed within the scope of the disclosure. Embodiments are not restricted to operation within certain specific data processing environments, but are free to operate within a plurality of data processing environments. Additionally, although embodiments have been described using a particular series of transactions and steps, it should be apparent to those skilled in the art that the scope of the present disclosure is not limited to the described series of transactions and steps. Various features and aspects of the above-described embodiments may be used individually or jointly.

Further, while embodiments have been described using a particular combination of hardware and software, it should be recognized that other combinations of hardware and software are also within the scope of the present disclosure. Embodiments may be implemented only in hardware, or only in software, or using combinations thereof. The various processes described herein can be implemented on the same processor or different processors in any combination. Accordingly, where components or services are described as being configured to perform certain operations, such configuration can be accomplished, e.g., by designing electronic circuits to perform the operation, by programming programmable electronic circuits (such as microprocessors) to perform the operation, or any combination thereof. Processes can communicate using a variety of techniques including but not limited to conventional techniques for inter process communication, and different pairs of processes may use different techniques, or the same pair of processes may use different techniques at different times.

The specification and drawings are, accordingly, to be regarded in an illustrative rather than a restrictive sense. It will, however, be evident that additions, subtractions, deletions, and other modifications and changes may be made thereunto without departing from the broader spirit and scope as set forth in the claims. Thus, although specific disclosure embodiments have been described, these are not intended to be limiting. Various modifications and equivalents are within the scope of the following claims.

The use of the terms “a” and “an” and “the” and similar referents in the context of describing the disclosed embodiments (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. The term “connected” is to be construed as partly or wholly contained within, attached to, or joined together, even if there is something intervening. Recitation of ranges of values herein are merely intended to serve as a shorthand method of referring individually to each separate value falling within the range, unless otherwise indicated herein and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate embodiments and does not pose a limitation on the scope of the disclosure unless otherwise claimed. No language in the specification should be construed as indicating any non-claimed element as essential to the practice of the disclosure.

Disjunctive language such as the phrase “at least one of X, Y, or Z,” unless specifically stated otherwise, is intended to be understood within the context as used in general to present that an item, term, etc., may be either X, Y, or Z, or any combination thereof (e.g., X, Y, and/or Z). Thus, such disjunctive language is not generally intended to, and should not, imply that certain embodiments require at least one of X, at least one of Y, or at least one of Z to each be present.

Preferred embodiments of this disclosure are described herein, including the best mode known for carrying out the disclosure. Variations of those preferred embodiments may become apparent to those of ordinary skill in the art upon reading the foregoing description. Those of ordinary skill should be able to employ such variations as appropriate and the disclosure may be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein.

All references, including publications, patent applications, and patents, cited herein are hereby incorporated by reference to the same extent as if each reference were individually and specifically indicated to be incorporated by reference and were set forth in its entirety herein.

In the foregoing specification, aspects of the disclosure are described with reference to specific embodiments thereof, but those skilled in the art will recognize that the disclosure is not limited thereto. Various features and aspects of the above-described disclosure may be used individually or jointly. Further, embodiments can be utilized in any number of environments and applications beyond those described herein without departing from the broader spirit and scope of the specification. The specification and drawings are, accordingly, to be regarded as illustrative rather than restrictive.

What is claimed is:

1. A computer-implemented method comprising:
accessing a medical image associated with a patient;
obtaining, based on a plurality of machine learning model prompts, a plurality of results from at least one machine learning model, the plurality of results comprising an image segmentation result for the medical image, an anomaly detection result for the medical image, and an image analysis summarization result for the medical image, wherein obtaining the plurality of results comprises:
using one or more first machine learning model prompts to obtain the image segmentation result for the medical image from the at least one machine learning model, at least one first machine learning model prompt of the one or more first machine learning model prompts comprising the medical image;
using one or more second machine learning model prompts to obtain the anomaly detection result for the medical image from the at least one machine learning model, at least one second machine learning model prompt of the one or more second machine learning model prompts comprising the image segmentation result; and
using one or more third machine learning model prompts to obtain the image analysis summarization result for the medical image from the at least one machine learning model, at least one third machine learning model prompt of the one or more third machine learning model prompts comprising the anomaly detection result; and
generating, based on the plurality of results, a graphical user interface page that provides the image analysis summarization result for the medical image.

2. The computer-implemented method of claim 1, further comprising:
generating, based at least on the medical image, a particular machine learning model prompt included in the one or more first machine learning model prompts;
providing the one or more first machine learning model prompts to the at least one machine learning model; and
receiving, from the at least one machine learning model, the image segmentation result, wherein the image segmentation result includes image segment data representing structural tissue variations depicted in the medical image.

3. The computer-implemented method of claim 1, further comprising:
generating, based at least on image segment data that is included in the image segmentation result, a particular machine learning model prompt included in the one or more second machine learning model prompts;
providing the one or more second machine learning model prompts to the at least one machine learning model; and
receiving, from the at least one machine learning model, the anomaly detection result, wherein the anomaly detection result includes a) one or more anomaly labels associated with a respective image segment in the image segment data, and b) text data describing a respective context associated with each of the one or more anomaly labels, the respective context based on a combination of each of the anomaly labels and electronic health data that is associated with the patient.

4. The computer-implemented method of claim 1, further comprising:
generating, based at least on one or more anomaly labels included in the anomaly detection result, a particular machine learning model prompt included in the one or more third machine learning model prompts, wherein each of the one or more anomaly labels is associated with a respective image segment that is based on the medical image;
providing the one or more third machine learning model prompts to the at least one machine learning model; and
receiving, from the at least one machine learning model, the image analysis summarization result, wherein the image analysis summarization result includes treatment recommendation data associated with the one or more anomaly labels, the treatment recommendation data describing a treatment option for a structural tissue variation represented by the respective image segment.

5. The computer-implemented method of claim 1, further comprising:
accessing an audio transcript data record associated with the patient;
obtaining, using the plurality of machine learning model prompts a dialogue segmentation result from at least one machine learning model, wherein the dialogue segmentation result identifies multiple participants in a dialogue recorded in the audio transcript data record;
obtaining, using the plurality of machine learning model prompts a) one or more individual summarization results comprising dialogue summarizations corresponding to each of the multiple participants identified in the dialogue segmentation result, and b) an aggregated summarization result comprising an additional dialogue summarization corresponding to the dialogue among the multiple participants identified in the dialogue segmentation result; and
modifying the graphical user interface page to further provide the one or more individual summarization result and the aggregated summarization result.

6. The computer-implemented method of claim 5, further comprising:
generating, based at least on the audio transcript data record, a particular machine learning model prompt included in the one or more machine learning model prompts;
providing the particular machine learning model prompt to the at least one machine learning model; and
receiving, from the at least one machine learning model, the dialogue segmentation result, wherein the dialogue segmentation result comprises a) identification data for the multiple participants in the dialogue recorded in the audio transcript data record, b) the dialogue, and c) the additional dialogue,
wherein the identification data is based on personnel data describing one or more of the multiple participants,
wherein the particular dialogue and the additional dialogue are determined based on a combination of the identification data and the audio transcript data record.

7. The computer-implemented method of claim 5, further comprising:
generating, based at least on the dialogue segmentation result, a particular machine learning model prompt included in the plurality of machine learning model prompts;
providing the particular machine learning model prompt to the at least one machine learning model; and
receiving, from the at least one machine learning model, one or more of the individual summarization result or the aggregated summarization result,
wherein the individual summarization result includes a respective individual summarization of respective dialogue of each participant of the multiple participants,
wherein the aggregated summarization result is based on a combination of the respective individual summarization for each participant.

8. A system comprising:
one or more processors; and
one or more computer-readable media storing instructions which, when executed by the one or more processors, cause the system to perform operations comprising:
accessing a medical image associated with a patient;
obtaining, based on a plurality of machine learning model prompts, a plurality of results from at least one machine learning model, the plurality of results comprising an image segmentation result for the medical image, an anomaly detection result for the medical image, and an image analysis summarization result for the medical image, wherein obtaining the plurality of results comprises:
using one or more first machine learning model prompts to obtain the image segmentation result for the medical image from the at least one machine learning model, at least one first machine learning model prompt of the one or more first machine learning model prompts comprising the medical image;
using one or more second machine learning model prompts to obtain the anomaly detection result for the medical image from the at least one machine learning model, at least one second machine learning model prompt of the one or more second machine learning model prompts comprising the image segmentation result; and
using one or more third machine learning model prompts to obtain the image analysis summarization result for the medical image from the at least one machine learning model, at least one third machine learning model prompt of the one or more third machine learning model prompts comprising the anomaly detection result; and
generating, based on the plurality of results, a graphical user interface page that provides the image analysis summarization result for the medical image.

9. The system of claim 8, wherein the operations further comprise:
generating, based at least on the medical image, a particular machine learning model prompt included in the one or more first machine learning model prompts;
providing the one or more first machine learning model prompts to the at least one machine learning model; and
receiving, from the at least one machine learning model, the image segmentation result, wherein the image segmentation result includes image segment data representing structural tissue variations depicted in the medical image.

10. The system of claim 8, wherein the operations further comprise:
generating, based at least on image segment data that is included in the image segmentation result, a particular machine learning model prompt included in the one or more second machine learning model prompts;
providing the one or more second machine learning model prompts to the at least one machine learning model; and
receiving, from the at least one machine learning model, the anomaly detection result, wherein the anomaly detection result includes a) one or more anomaly labels associated with a respective image segment in the image segment data, and b) text data describing a respective context associated with each of the one or more anomaly labels, the respective context based on a combination of each of the anomaly labels and electronic health data that is associated with the patient.

11. The system of claim 8, wherein the operations further comprise:
generating, based at least on one or more anomaly labels included in the anomaly detection result, a particular machine learning model prompt included in the one or more third machine learning model prompts, wherein each of the one or more anomaly labels is associated with a respective image segment that is based on the medical image;
providing the one or more third machine learning model prompts to the at least one machine learning model; and
receiving, from the at least one machine learning model, the image analysis summarization result, wherein the image analysis summarization result includes treatment recommendation data associated with the one or more anomaly labels, the treatment recommendation data describing a treatment option for a structural tissue variation represented by the respective image segment.

12. The system of claim 8, wherein the operations further comprise:
accessing an audio transcript data record associated with the patient;
obtaining, using the plurality of machine learning model prompts a dialogue segmentation result from at least one machine learning model, wherein the dialogue segmentation result identifies multiple participants in a dialogue recorded in the audio transcript data record;
obtaining, using the plurality of machine learning model prompts a) one or more individual summarization results comprising dialogue summarizations corresponding to each of the multiple participants identified in the dialogue segmentation result, and b) an aggregated summarization result comprising an additional dialogue summarization corresponding to the dialogue among the multiple participants identified in the dialogue segmentation result; and
modifying the graphical user interface page to further provide the one or more individual summarization result and the aggregated summarization result.

13. The system of claim 12, wherein the operations further comprise:
generating, based at least on the audio transcript data record, a particular machine learning model prompt included in the one or more machine learning model prompts;
providing the particular machine learning model prompt to the at least one machine learning model; and
receiving, from the at least one machine learning model, the dialogue segmentation result, wherein the dialogue segmentation result comprises a) identification data for the multiple participants in the dialogue recorded in the audio transcript data record, b) the dialogue, and c) the additional dialogue,
wherein the identification data is based on personnel data describing one or more of the multiple participants,
wherein the particular dialogue and the additional dialogue are determined based on a combination of the identification data and the audio transcript data record.

14. The system of claim 12, wherein the operations further comprise:
generating, based at least on the dialogue segmentation result, a particular machine learning model prompt included in the plurality of machine learning model prompts;
providing the particular machine learning model prompt to the at least one machine learning model; and
receiving, from the at least one machine learning model, one or more of the individual summarization result or the aggregated summarization result,
wherein the individual summarization result includes a respective individual summarization of respective dialogue of each participant of the multiple participants,
wherein the aggregated summarization result is based on a combination of the respective individual summarization for each participant.

15. One or more non-transitory computer-readable media storing instructions which, when executed by one or more processors, cause the one or more processors to perform operations comprising:
accessing a medical image associated with a patient;
obtaining, based on a plurality of machine learning model prompts, a plurality of results from a multimodal generative model, the plurality of results comprising an image segmentation result for the medical image, an anomaly detection result for the medical image, and an image analysis summarization result for the medical image, wherein obtaining the plurality of results comprises:
using one or more first machine learning model prompts to obtain the image segmentation result for the medical image from the multimodal generative model, at least one first machine learning model prompt of the one or more first machine learning model prompts comprising the medical image;
using one or more second machine learning model prompts to obtain the anomaly detection result for the medical image from the multimodal generative model, at least one second machine learning model prompt of the one or more second machine learning model prompts comprising the image segmentation result; and
using one or more third machine learning model prompts to obtain the image analysis summarization result for the medical image from the multimodal generative model, at least one third machine learning model prompt of the one or more third machine learning model prompts comprising the anomaly detection result; and
generating, based on the plurality of results, a graphical user interface page that provides the image analysis summarization result for the medical image.

16. The one or more non-transitory computer-readable media of claim 15, wherein the operations further comprise:
generating, based at least on the medical image, a particular machine learning model prompt included in the one or more first machine learning model prompts;
providing the one or more first machine learning model prompts to the multimodal generative model; and
receiving, from the multimodal generative model, the image segmentation result, wherein the image segmentation result includes image segment data representing structural tissue variations depicted in the medical image.

17. The one or more non-transitory computer-readable media of claim 15, wherein the operations further comprise:
generating, based at least on image segment data that is included in the image segmentation result, a particular machine learning model prompt included in the one or more second machine learning model prompts;
providing the one or more second machine learning model prompts to the multimodal generative model; and
receiving, from the at least one machine learning model, the anomaly detection result, wherein the anomaly detection result includes a) one or more anomaly labels associated with a respective image segment in the image segment data, and b) text data describing a respective context associated with each of the one or more anomaly labels, the respective context based on a combination of each of the anomaly labels and electronic health data that is associated with the patient.

18. The one or more non-transitory computer-readable media of claim 15, wherein the operations further comprise:
generating, based at least on one or more anomaly labels included in the anomaly detection result, a particular machine learning model prompt included in the one or more third machine learning model prompts, wherein each of the one or more anomaly labels is associated with a respective image segment that is based on the medical image;
providing the one or more third machine learning model prompts to the multimodal generative model; and
receiving, from the at least one machine learning model, the image analysis summarization result, wherein the image analysis summarization result includes treatment recommendation data associated with the one or more anomaly labels, the treatment recommendation data describing a treatment option for a structural tissue variation represented by the respective image segment.

19. The one or more non-transitory computer-readable media of claim 15, wherein the operations further comprise:
accessing an audio transcript data record associated with the patient;
obtaining, using the plurality of machine learning model prompts a dialogue segmentation result from at least one machine learning model, wherein the dialogue segmentation result identifies multiple participants in a dialogue recorded in the audio transcript data record;
obtaining, using the plurality of machine learning model prompts a) one or more individual summarization results comprising dialogue summarizations corresponding to each of the multiple participants identified in the dialogue segmentation result, and b) an aggregated summarization result comprising an additional dialogue summarization corresponding to the dialogue among the multiple participants identified in the dialogue segmentation result; and
modifying the graphical user interface page to further provide the one or more individual summarization result and the aggregated summarization result.

20. The one or more non-transitory computer-readable media of claim 19, wherein the operations further comprise:
generating, based at least on the audio transcript data record, a particular machine learning model prompt included in the one or more machine learning model prompts;
providing the particular machine learning model prompt to the at least one machine learning model; and
receiving, from the at least one machine learning model, the dialogue segmentation result, wherein the dialogue segmentation result comprises a) identification data for the multiple participants in the dialogue recorded in the audio transcript data record, b) the dialogue, and c) the additional dialogue,
wherein the identification data is based on personnel data describing one or more of the multiple participants,
wherein the particular dialogue and the additional dialogue are determined based on a combination of the identification data and the audio transcript data record.
